(19) United States
(12) Patent Application Publication
(10) Pub. No.: US 2004/0029239 A1
(43) Pub. Date: Feb. 12, 2004

(54) Method of producing prenyl alcohols

(75) Inventors: Chikara Ohto, Toyota-shi (JP); Shusei Obata, Nagoya-shi (JP)

Correspondence Address: KENYON & KENYON, Suite 700, 1500 K Street, N.W., Washington, DC, 20005-1257, US

(73) Assignee: TOYOTA JIDOSHA KABUSHIKI KAISHA, Toyota-shi (JP)

(21) Appl. No.: 10/462,698

(22) Filed: Jun. 17, 2003

Related U.S. Application Data
(63) Continuation of application No. PCT/JP01/11213, filed on Dec. 20, 2001.

(30) Foreign Application Priority Data
Dec. 28, 2000 (JP) 2000-401701

(51) Int. Cl. C12P 7/04

(52) U.S. Cl. 435/157

(57) ABSTRACT

The present invention provides a method of producing a prenyl alcohol, comprising creating a recombinant obtained by transferring into a host a recombinant DNA for expression or a DNA fragment for genomic integration each comprising:

(i) a hydroxymethylglutaryl-CoA reductase gene, an isopentenyl-diphosphate Δ-isomerase gene or a farnesyl-diphosphate synthase gene, or a mutant of any one of these genes,

(ii) a transcription promoter, and

(iii) a transcription terminator;

culturing the recombinant;

and recovering the prenyl alcohol from the resultant culture.

### TECHNICAL FIELD

The present invention relates to a method of producing prenyl alcohols.

### BACKGROUND ART

The biosynthesis of terpenoids (isoprenoids) begins with the synthesis of geranyl diphosphate (GPP; C₁₀), farnesyl diphosphate (FPP; C₁₅) and geranylgeranyl diphosphate (GGPP; C₂₀), which are straight chain prenyl diphosphates, through the sequential condensation reactions of isopentenyl diphosphate (IPP; C₅) with an allylic diphosphate substrate (FIG. 1). In FIG. 1, the abbreviations and words in boxes represent enzymes. Specifically, hmgR represents hydroxymethylglutaryl-CoA reductase; GGPS represents GGPP synthase; and FPS represents FPP synthase.

Among prenyl diphosphates, FPP is the most significant biosynthetic intermediate. It is a precursor for the synthesis of tremendous kinds of terpenoids, e.g. steroids including ergosterol (provitamin D₂), the side chains of quinone (vitamin K; VK), sesquiterpenes, squalene (SQ), the anchor molecules of farnesylated proteins, and natural rubber.

GGPP is also a key biosynthetic intermediate in vivo, and is essential for the biosynthesis of such compounds as retinol (vitamin A; VA), β-carotene (provitamin A), phylloquinone (vitamin K₁; VK₁), tocopherols (vitamin E; VE), the anchor molecules of geranylgeranylated proteins, the side chain of chlorophyll, gibberellins, and the ether lipid of Archaea.

Farnesol (FOH; C₁₅) and nerolidol (NOH; C₁₅), which are alcohol derivatives of FPP, and geranylgeraniol (GGOH; C₂₀), which is an alcohol derivative of GGPP, are known as fragrant substances in essential oils used as the ingredients of perfumes. FOH, NOH and GGOH are also important as the starting materials for the synthesis of various compounds (including the above-mentioned vitamins) useful as pharmacological agents (FIG. 1).

It is desired to establish a system in which a pure product of the so-called active-type prenyl alcohol, not a mixture containing isomers, can be produced in a large quantity.

Although it had been believed that all the biosynthesis of IPP is performed via the mevalonate pathway (a pathway in which IPP is synthesized from acetyl-CoA through mevalonate), M. Rohmer et al. elucidated a novel pathway for IPP synthesis using bacteria at the end of 1980's. This is called non-mevalonate pathway or DXP (1-deoxyxylulose 5-phosphate) pathway, in which IPP is synthesized from glyceraldehyde-3-phosphate and pyruvate through 1-deoxyxylulose 5-phosphate.

FOH and NOH are currently produced by chemical synthesis except for small amounts of them prepared from natural products such as essential oils. Chemically synthesized FOH and NOH generally have the same carbon skeletons, but they are obtained as mixtures of (E) type (trans type) and (Z) type (cis type) in double bond geometry. (E, E)-FOH or (E)-NOH, both of which are of (all-E) type, is the form synthesized in metabolic pathways in organisms and is industrially valuable. In order to obtain (E, E)-FOH or (E)-NOH in a pure form, refining by column chromatography, high precision distillation, etc. is necessary. However, it is difficult to carry out high precision distillation of FOH, a thermolabile allyl alcohol, or its isomer FOH. Also, the refining of these substances by column chromatography is not suitable in industrial practice since it requires large quantities of solvent and column packings as well as complicated operations of analyzing and recovering serially eluting fractions and removing the solvent; thus, this method is complicated and requires high cost. Under circumstances, it is desired to establish a method of biosynthesis of (E, E)-FOH (hereinafter, just referred to as “FOH”) by controlling the production of (E)- and (Z)-geometrical isomers or by utilizing the repeat structure of reaction products. However, such a method has not been established yet. The substrates for FOH synthesis are provided via the mevalonate pathway in cells of, for example, Saccharomyces cerevisiae, a budding yeast. However, even when HMG-CoA reductase that is believed to be a key enzyme for FOH synthesis was used, it has only been discovered that the use of the reductase increases squalene synthesis ability (Japanese Unexamined Patent Publication No. 5-192184; Donald et al., (1997) Appl. Environ. Microbiol. 63, 3341-3344). Further, even when a squalene synthase gene-deficient strain of a special budding yeast that had acquired sterol intake ability was cultured, accumulation of 1.3 mg of FOH per liter of culture broth was only revealed (Chambon et al., (1990) Curr. Genet. 18, 41-46); no method of biosynthesis of (E)-NOH (hereinafter, just referred to as “NOH”) has been known.

### DISCLOSURE OF THE INVENTION

It is an object of the invention to provide a method for producing a prenyl alcohol by culturing a recombinant prepared by transferring into a host cell a recombinant DNA for expression comprising an HMG-CoA reductase gene, an IPP Δ-isomerase gene or an FPP synthase gene, or a mutant of any one of these genes.

As a result of intensive and extensive researches toward solution of the above problems, the present inventors attempted to develop a prenyl alcohol production system by introducing into a host a gene of an enzyme involved in prenyl diphosphate synthesis. As the host, an unicellular eucaryote, in particular, yeast or procaryotes (such as bacterium, in particular, E. coli) that had been widely used in the fermentation industry from old times, that carries out the synthesis of prenyl diphosphate via the mevalonate pathway or DXP pathway; and that can be subjected to various genetic engineering techniques was used. In order to construct systems with which a gene of an enzyme involved in prenyl diphosphate synthesis (e.g., HMG-CoA reductase gene) in yeast can be expressed artificially in a host cell, expression shuttle vectors were created which comprised a constitutive or inducible transcription promoter and various auxotrophic markers. Then, a gene of interest or a mutant thereof was inserted into these vectors, which were then introduced into various host cells. The inventors have succeeded in obtaining NOH or FOH from the culture of the resultant recombinant. Thus, the above-mentioned object has been achieved, and the present invention has been completed. When E. coli was used as a host, a gene of an enzyme involved in prenyl diphosphate synthesis (e.g., FPP synthase gene or IPPΔ-isomerase gene) was introduced into the host cell using a conventional vector. Then, FOH was obtained from the culture of the resultant recombinant after dephosphorylation. Thus, the above-mentioned object has been achieved, and the present invention has been completed.

The present invention relates to a method of producing a prenyl alcohol(s), comprising creating a recombinant obtained by introducing into a host a recombinant DNA(s) for expression or a DNA fragment(s) for genomic integration each comprising:

(i) a hydroxymethylglutaryl-CoA reductase gene, an isopentenyl-diphosphate Δ-isomerase gene or a farnesyl-diphosphate synthase gene, or a mutant of any one of these genes,

(ii) a transcription promoter, and

(iii) a transcription terminator;

culturing the recombinant; and recovering the prenyl alcohol(s) from the resultant culture. Specific examples of the prenyl alcohol include C₁₅ prenyl alcohols such as FOH or NOH. Specific examples of the HMG-CoA reductase gene and mutant thereof include a gene encoding the amino acid sequence as shown in SEQ ID NO: 2, 4 or 6, or a deletion mutant thereof. For example, an HMG-CoA reductase gene comprising one nucleotide sequence selected from the group consisting of SEQ ID NOS: 1, 3, 5 and 7-16 may be given. Specific examples of the FPP synthase gene or mutant thereof include a gene encoding the amino acid sequence as shown in SEQ ID NO: 76, 78, 80, 82 or 84. For example, an FPP synthase gene comprising one nucleotide sequence selected from the group consisting of SEQ ID NOS: 75, 77, 79, 81 and 83 may be given. Specific examples of the IPPA-isomerase gene or mutant thereof include a gene encoding the amino acid sequence as shown in SEQ ID NO: 86. For example, an IPPA-isomerase gene comprising the nucleotide sequence as shown in SEQ ID NO: 85 may be given. As the transcription promoter, one selected from the group consisting of ADH1 promoter, TDH3 (GAP) promoter, PGK1promoter, TEF2 promoter, GAL1 promoter and tac promoter may be used. Other transcription promoters may also be used which are functionally equivalent to these promoters in activity. As the transcription terminator, ADH1 terminator or CYC1 terminator may be used. Other transcription terminators may also be used which are functionally equivalent to these terminators in activity. As the host, yeast may be used, e.g. budding yeast such as Saccharomyces cerevisia. Specific examples of preferable S. cerevisiae strains include A451, YPH499, YPH500, W303-1A and W303-1B, or strains derived therefrom. Alternatively, a bacterium, e.g. Escherichia coli may be used. Specific examples of preferable E. coli strains include JM109 or strains derived therefrom.

According to the present invention, it is possible to produce a prenyl alcohol such as NOH or FOH at a concentration that cannot be achieved by merely culturing the untransformed host cell (at least 0.05 mg/L medium).

Further, the present invention relates to a recombinant obtained by transferring into a host a recombinant DNA for expression or a DNA fragment for genomic integration each comprising:

(i) a hydroxymethylglutaryl-CoA reductase gene, an isopentenyl-diphosphate Δ-isomerase gene or a farnesyl-diphosphate synthase gene, or a mutant of any one of these genes,

(ii) a transcription promoter, and

(iii) a transcription terminator,

the recombinant being capable of producing at least 0.05 mg/L of FOH or NOH. Specific examples of the host, the promoter and the terminator are the same as described above.

Hereinbelow, the present invention will be described in detail. The present specification encompasses the contents described in the specification and the drawings of Japanese Patent Application No. 2000-401701 based on which the present application claims priority.

The inventors have attempted to develop a system with which an active-type prenyl alcohol (i.e., (all-E)-prenyl alcohol) can be produced in vivo, by using metabolic engineering techniques. Generally, FPP is synthesized by the catalytic action of farnesyl-diphosphate synthase (FPS) from IPP and DMAPP (3,3-dimethylallyl diphosphate) as substrates. Usually, this reaction does not proceed toward the synthesis of FOH, but proceeds toward the synthesis of squalene by squalene synthase, the synthesis of GGPP by geranygeranyl-diphosphate synthase, the synthesis of hexaprenyl diphosphate by hexaprenyl-diphosphate synthase, and so on (FIG. 1). In the present invention, transformant cells capable of producing not the usually expected squalene or major final products (sterols) but prenyl alcohols such as NOH and FOH not indicated in conventional metabolic pathway maps have been obtained by introducing into host cells an HMG-CoA reductase gene, FPP synthase gene or IPP Δ-isomerase gene that are believed to be involved in the activation of prenyl diphosphate synthesis via two different, independent pathways (the mevalonate pathway and DXP pathway) depending on organisms. Thus, biological, mass-production systems for prenyl alcohols have been developed. Furthermore, deletion mutants of HMG-CoA reductase gene with various patterns of deletions (FIG. 2) have been introduced into hosts in such a manner that the genes come under the control of a transcription promoter; or mutants of FPP synthase with amino acid substitutions have been introduced into hosts. Thus, biological, mass-production systems for the above-mentioned prenyl alcohols have been developed.

1. Preparation of Recombinant DNAs for Expression or DNA Fragments for Genomic Integration

In the present invention, the recombinant DNA for expression used in the transformation of hosts may be obtained by ligating or inserting a transcription promoter DNA and a transcription terminator DNA into a gene of interest to be expressed. Specifically, the gene to be expressed may be, for example, an HMG-CoA reductase genes (e.g., HMG1), Escherichia coli FPP synthase gene ispA, Bacillus stearothermophilus FPP synthase gene or IPPΔ-isomerase gene idi (ORF182) (hereinafter, referred to as an “HMG-CoA reductase gene or the like”). These genes can be isolated by cloning techniques using PCR or commercial kits.

It is also possible to prepare in advance a gene expression cassette comprising an HMG-CoA reductase gene or the like to which a transcription promoter and a transcription terminator have been ligated, and to incorporate the cassette into a vector. The ligation of the promoter and the terminator may be performed in any order. However, the promoter is ligated upstream of the HMG-CoA reductase gene or the like, and the terminator downstream of the gene. Alternatively, in the present invention, an HMG-CoA reductase gene or the like, a transcription promoter and a transcription terminator may be incorporated into an appropriate DNA, e.g a vector, in succession. If the direction of transcription is properly considered, the incorporation may be performed in any order.

The DNA used for this purpose is not particularly limited as long as it may be retained in host cells hereditarily. Specific examples of DNA that may be used include plasmid DNA, bacteriophage, retrotransposon DNA and artificial chromosomal DNA (YAC: yeast artificial chromosome). With respect to recombinant DNA fragments for the gene expression by genomic integration, replication ability is not necessarily required in that DNA. The DNA fragments prepared by PCR or chemical synthesis may also be used.

Specific examples of useful plasmid DNA include YCp-type E. coli-yeast shuttle vectors such as pRS413, pRS414, pRS415, pRS416, YCp50, pAUR112 or pAUR123; YEp-type E. coli-yeast shuttle vectors such as pYES2 or YEp13; YIp-type E. coli-yeast shuttle vectors such as pRS403, pRS404, pRS405, pRS406, pAUR101 or pAUR135; E. coli-derived plasmids such as ColE plasmids (e.g., pBR322, pBR325, pUC18, pUC19, pUC118, pUC119, pTV118N, pTV119N, pBluescript, pHSG298, pHSG396 or pTrc99A), p15A plasmids (e.g., pACYC177 or pACYC184) and pSCO1 plasmids (e.g., pMW118, pMW119, pMW218 or pMW219); and Bacillus subtilis-derived plasmids (e.g., pUB110, pTP5). Specific examples of useful phage DNA include λ phage (Charon4A, Charon21A, EMBL3, EMBL4, λgt10, λgt11, λZAP), φ174, M13mp18 and M13mp19. Specific examples of useful retrotransposon DNA include Ty factor. Specific examples of YAC vectors include pYACC2.

When recombinant DNAs are introduced into hosts, selection marker genes are used in many cases. However, the use of the marker genes are not necessarily required if there is an appropriate assay to select recombinants.

As the transcription promoter, a constitutive promoter or an inducible promoter may be used. The “constitutive promoter” means a transcription promoter of a gene involved in a major metabolic pathway. Such a promoter is believed to have transcription activity under any growth conditions. The “inducible promoter” means a promoter that has transcription activity only under specific growth conditions and whose activity is suppressed under other growth conditions.

Any transcription promoter may be used as long as it has activity in hosts such as yeast. For example, GAL1 promoter, GAL10 promoter, TDH3 (GAP) promoter, ADH1 promoter, PGK1 promoter or TEF2 promoter may be used to direct expression in yeast. To direct expression in E. coli, trp promoter, lac promoter, trc promoter or tac promoter may be used, for example.

The recombinant DNA may further comprise cis-elements such as an enhancer, a splicing signal, a poly A addition signal, selection markers, or the like, if desired. Specific examples of useful selection markers include marker genes such as URA3, LEU2, TRP1 and HIS3 that have non-auxotrophic phenotypes as indicators, and drug resistance genes such as Amp^{r}, Tet^{r}, Cm^{r}, Km^{r} and AUR1-C.

A transcription terminator derived from any gene may be used as long as it has activity in hosts such as yeast. For example, ADH1 terminator or CYC1 terminator may be used to direct the expression in yeast. To direct the expression in E. coli, rrnB terminator may be used, for example. It is also possible to incorporate an SD sequence (typically, 5′-AGGAGG-3′) upstream of the initiation codon of the gene of a bacterium (e.g., E. coli) as a ribosome binding site for translation.

Expression vectors prepared in the present invention as recombinant DNAs for gene transfer may be designated and identified by indicating the name of the gene after the name of the plasmid used, unless otherwise noted. For example, when HMG1 gene has been ligated to plasmid pRS434GAP having TDH3 (GAP) promoter, the resultant plasmid is expressed as “pRS434GAP-HMG1”. Except for special cases, this notational system applies to other expression vectors comprising other plasmids, promoters and genes.

In the present invention, an HMG-CoA reductase gene or the like may be a mutant in which a part of its regions (2217 nucleotides at the maximum) has been deleted, or a mutant that has deletion, substitution or addition of one or several to ten-odd nucleotides in the nucleotide sequence of a wild-type gene or a deletion mutant thereof. With respect to amino acid sequences, an HMG-CoA reductase may be a deletion mutant in which 739 amino acids at the maximum have been deleted in the amino acid sequence of a wild-type HMG-CoA reductase (SEQ ID NO: 2), or it may be a mutant that has deletion, substitution or addition of one or several (e.g, one to ten, preferably one to three) amino acids in the amino acid sequence of the wild-type enzyme or a deletion mutant thereof. Specifically, an HMG-CoA reductase gene may be a wild-type gene or a deletion mutant thereof as shown in FIG. 2B. Also, the amino acid sequence encoded by such a gene may have site-specific substitution(s) at one to ten sites as a result of nucleotide substitution(s), for example, as shown in FIG. 2A. An FPP synthase gene may also be a mutant that has deletion, substitution or addition of one or several to ten-odd nucleotides. Specifically, various mutant genes (SEQ ID NOS: 79, 81 and 83) each of which has substitution of five nucleotides in a wild-type FPP synthase gene (SEQ ID NO: 77) may be used. These mutant genes encode mutant enzymes in which the 79th amino acid residue Tyr of the wild-type FPP synthase (SEQ ID NO: 78) has been changed to Asp (SEQ ID NO: 80), Glu (SEQ ID NO: 82) or Met (SEQ ID NO: 84), respectively.

Substitution mutations of nucleotides that occur in DNA fragments obtained by amplifying wild-type DNA by PCR (polymerase chain reaction) using a DNA polymerase of low fidelity, such as Taq DNA polymerase, are called “PCR errors”. In the present invention, for example, an HMG-CoA reductase gene in which encoded polypeptide has substitution mutations attributable to those nucleotide substitutions resulted from PCR errors when a wild-type HMG-CoA reductase gene (SEQ ID NO: 1) was used as a template may also be used. This HMG-CoA reductase gene is called “HMG1′”. An embodiment of nucleotide substitutions resulted from PCR errors when the wild-type HMG-CoA reductase gene (SEQ ID NO: 1) was used as a template is shown in FIG. 2A. HMG1′ has the nucleotide sequence as shown in SEQ ID NO: 3, and the amino acid sequence encoded thereby is shown in SEQ ID NO: 4. In FIG. 2A, the mutations of nucleotides are expressed in the following order: the relevant nucleotide before substitution (in one letter abbreviation), the position of this nucleotide when the first nucleotide in the initiation codon of the HMG-CoA reductase gene is taken as position 1, and the nucleotide after substitution (in one letter abbreviation). The mutations of amino acids contained in the amino acid sequence of the PCR error-type HMG-CoA reductase are expressed in the following order: the relevant amino acid residue before substitution (in one letter abbreviation), the position of this amino acid in the HMG-CoA reductase, and the amino acid residue after substitution (in one letter abbreviation). Further, the PCR error-type nucleotide sequence described above may be corrected partially by techniques such as site-directed mutagenesis. Such a corrected HMG-CoA reductase gene may also be used in the invention. Further, those HMG-CoA reductase genes (including PCR error-type) may also be used in the invention that encode deletion mutants in which predicted transmembrane domains are deleted. For example, FIG. 2B shows examples of HMG1Δ genes that are deletion mutants of the PCR error-type HMG-CoA reductase gene HMG1′. In FIG. 2B, the upper most row represents HMG1′ gene without deletion. The portion indicated with thin solid line (—) is the deleted region. Table 1 below shows which region of HMG1′ gene (SEQ ID NO: 3) has been deleted for each deletion mutant. Deletion mutants of HMG1′ are expressed as “HMG1Δxxy” according to the deletion pattern, in which “xx” represents the deletion pattern and “y” a working number (any numerical figure). In FIG. 2B, “Δ026” is shown as one example of HMG1Δ02y. (Likewise, examples of other deletion patterns are also shown.)

TABLE 1
Embodiment of Deletions

| | | | | | | |
|---|---|---|---|---|---|---|
| Designation | | | | Deletion of | | |
| of | | | | Predicted | | |
| Deletion | | | | Transmembrane | | Sequence after |
| Mutant | Primer 1 | Primer 2 | Plasmid | Domains | Deleted Region | Deletion |

| | | | | | | | |
|---|---|---|---|---|---|---|---|
| HMG1 Δ 02y | HMG1(558-532) | HMG1(799-825) | pYHMG02X | #2-#3 | Nucleotide | 559-798 | SEQ ID NO: 7 |
| | | | | positions | | | |
| HMG1 Δ 04y | HMG1(1191-1165) | HMG1(1267-1293) | pYHMG04X | #6 | Nucleotide | 1192-1266 | SEQ ID NO: 8 |
| | | | | | positions | | |
| HMG1 Δ 05y | HMG1(1380-1354) | HMG1(1573-1599) | pYHMG05X | #7 | Nucleotide | 1381-1572 | SEQ ID NO: 9 |
| | | | | | positions | | |
| HMG1 Δ 06y | HMG1(558-532) | HMG1(1267-1293) | pYHMG06X | #2-#6 | Nucleotide | 559-1266 | SEQ ID NO: 10 |
| | | | | | positions | | |
| HMG1 Δ 07y | HMG1(558-532) | HMG1(1573-1599) | pYHMG07X | #2-#7 | Nucleotide | 559-1572 | SEQ ID NO: 11 |
| | | | | | positions | | |
| HMG1 Δ 08y | HMG1(27-1) | HMG1(1573-1599) | pYHMG08X | #1-#7 | Nucleotide | 27-1572 | SEQ ID NO: 12 |
| | | | | | positions | | |
| HMG1 Δ 10y | HMG1(27-1) | HMG1(1816-1842) | pYHMG10X | #1-#7(−605 aa) | Nucleotide | 27-1815 | SEQ ID NO: 13 |
| | | | | | positions | | |
| HMG1 Δ 11y | HMG1(27-1) | HMG1(1891-1917) | pYHMG11X | #1-#7(−631 aa) | Nucleotide | 27-1890 | SEQ ID NO: 14 |
| | | | | | positions | | |
| HMG1 Δ 12y | HMG1(27-1) | HMG1(1990-2016) | pYHMG12X | #1-#7(−663 aa) | Nucleotide | 27-1989 | SEQ ID NO: 15 |
| | | | | | positions | | |
| HMG1 Δ 13y | HMG1(27-1) | HMG1(2218-2244) | pYHMG13X | #1-#7(−739 aa) | Nucleotide | 27-2217 | SEQ ID NO: 16 |
| | | | | | positions | | |

| | Primer Sequence | |
|---|---|---|
| HMG1(27-1) | 5′TTT CAG TCC CTT GAA TAG CGG CGG CAT 3′ | SEQ ID NO: 38 |
| HMG1(558-532) | 5′GTC TGC TTG GGT TAC ATT TTC TGA AAA 3′ | SEQ ID NO: 39 |
| HMG1(799-825) | 5′CAC AAA ATC AAG ATT GCC CAG TAT GCC 3′ | SEQ ID NO: 40 |
| HMG1(1191-1165) | 5′AGA AGA TAC GGA TTT CTT TTC TGC TTT 3′ | SEQ ID NO: 41 |
| HMG1(1267-1293) | 5′AAC TTT GGT GCA AAT TGG GTC AAT GAT 3′ | SEQ ID NO: 42 |
| HMG1(1380-1354) | 5′TTG CTC TTT AAA GTT TTC AGA GGC ATT 3′ | SEQ ID NO: 43 |
| HMG1(1573-1599) | 5′CAT ACC AGT TAT ACT GCA GAC CAA TTG 3′ | SEQ ID NO: 44 |
| HMG1(1816-1842) | 5′GCA TTA TTA AGT AGT GGA AAT ACA ATT 3′ | SEQ ID NO: 4S |
| HMG1(1891-1917) | 5′CCT TTG TAC GCT TTG GAG AAA AAA TTA 3′ | SEQ ID NO: 46 |
| HMG1(1990-2016) | 5′TCT GAT CGT TTA CCA TAT AAA AAT TAT 3′ | SEQ ID NO: 47 |
| HMG1(2218-2244) | 5′TTG GAT GGT ATG ACA AGA GGC CCA GTA 3′ | SEQ ID NO: 48 |

2. Preparation of Recombinants

The recombinant of the invention can be obtained by introducing into a host the recombinant DNA of the invention in such a manner that the HMG-CoA reductase gene or the like (including various mutants; the same applies to the rest of the present specification unless otherwise noted) can be expressed. The host used in the invention is not particularly limited. Any host may be used as long as it can produce a prenyl alcohol(s). Preferably, E. coli or yeast is used.

In the present invention, the recombinant DNA comprising a promoter, an HMG-CoA reductase gene or the like, and a terminator may be introduced into fungi including unicellular eucaryotes such as yeast; procaryotes such as E. coli; animal cells; plant cells; etc. to obtain recombinants.

Fungi useful in the invention include Myxomycota, Phycomycetes, Ascomycota, Basidiomycota, and Fungi Imperfecti. Among fungi, some unicellular eucaryotes are well known as yeast that is important in industrial applicability. For example, yeast belonging to Ascomycota, yeast belonging to Basidiomycota, or yeast belonging to Fungi Imperfecti may be enumerated. Specific examples of yeast include yeast belonging to Ascomycota, in particular, budding yeast such as Saccharomyces cerevisiae (known as Baker's yeast), Candida utilis or Pichia pastris; and fission yeast such as Shizosaccharomyces pombe. The yeast strain is not particularly limited as long as it can produce a prenyl alcohol(s). In the case of S. cerevisiae, specific examples of useful strains include A451, EUG8, EUG12, EUG27, YPH499, YPH500, W303-1A, W303-1B and AURGG101 strains as shown below. As a method for introducing the recombinant DNA into yeast, such method as electroporation, the spheroplast method, or the lithium acetate method may be employed.

A451 (ATCC200589; MATa can1 leu2 trp1 ura3 aro7)

YPH499 (ATCC76625; MATa ura3-52 lys2-801 ade2-101 trp1-Δ63 his3-Δ200 leu2-Δ1; Stratagene, La Jolla, Calif.)

YPH500 (ATCC76626; MATa ura3-52 lys2-801 ade2-101 trp1-Δ63 his3-Δ200 leu2-Δ1; Stratagene)

W303-1A (MATa leu2-3 leu2-112 his3-11 ade2-1 ura3-1 trp1-1 can1-100)

W303-1B (MATa leu2-3 leu2-112 his3-11 ade2-1 ura3-1 trp1-1 can1-100)

AURGG101(A451, aur1::AUR1-C)

EUG8 (A451, ERG9p::URA3-GAL1p)

EUG12 (YPH499, ERG9p::URA3-GAL1p)

EUG27 (YPH500, ERG9p::URA3-GAL1p)

As prokaryotes, archaea and bacteria may be enumerated. As archaea, methane producing microorganisms such as Metanobacterium; halophilic microorganisms such as Halobacterium, thermophilic acidophilic microorganisms such as Sulfolobus, may be enumerated. As bacteria, various Gram-negative or Gram-positive bacteria that are highly valuable in industrial or scientific applicability may be enumerated, e.g. Escherichia such as E. coli, Bacillus such as B. subtilis or B. brevis, Pseudomonas such as P. putida, Agrobacterium such as A. tumefaciens or A. rhizogenes, Corynebacterium such as C. glutamicum, Lactobacillus such as L. plantarum, and Actinomycetes such as Actinomyces or Streptmyces.

When a bacterium such as E. coli is used as a host, the recombinant DNA of the invention is preferably not only capable of autonomous replication in the host but also composed of a promoter, an SD sequence as a ribosome RNA binding site, and the gene of the invention. A transcription terminator may also be inserted appropriately. The recombinant DNA may also contain a gene that controls the promoter. Specific examples of E. coli strains include, but are not limited to, BL21, DH5a, HB101, JM101, MBV1184, TH2, XL1-Blue and Y-1088. As the transcription promoter, any promoter may be used as long as it can direct the expression of a gene in a host such as E. coli. For example, an E. coli- or phage-derived promoter such as trp promoter, lac promoter, P_{L} promoter or P_{R} promote may be used. An artificially altered promoter such as tac promoter may also be used. As a method for introducing the recombinant DNA into a bacterium, any method of DNA transfer into bacteria may be used. For example, a method using calcium ions, electroporation, or a method using a commercial kit may be employed.

Whether the gene of the invention has been transferred into the host cell or not can be confirmed by such methods as PCR or Southern blot hybridization. For example, DNA is prepared from the resultant recombinant, designed a primer(s) specific to the introduced DNA and subjected to PCR. Subsequently, the amplified product is subjected to agarose gel electrophoresis, polyacrylamide gel electrophoresis or capillary electrophoresis, followed by staining with ethidium bromide, SYBR Green solution or the like, or detection of DNA with a UV detector. Thus, by detecting the amplified product as a single band or peak, the introduced DNA can be confirmed. Alternatively, PCR may be performed using a primer(s) labeled with a fluorescent dye or the like to detect the amplified product.

3. Production of Prenyl Alcohols

In the present invention, a prenyl alcohol(s) can be obtained by culturing the above-described recombinant comprising a transferred HMG-CoA reductase gene or the like, and recovering the prenyl alcohol(s) from the resultant culture. The term “culture” used herein means any of the following materials: culture supernatant, cultured cells or microorganisms per se, or disrupted products from cultured cells or microorganisms. The recombinant of the invention is cultured by conventional methods used in the culture of hosts. As the prenyl alcohol, C₁₅prenyl alcohols such as farnesol (FOH) or nerolidol (NOH) may be enumerated. These prenyl alcohols are accumulated in the culture independently or as a mixture.

As a medium to culture the recombinant obtained from a microorganism host, either a natural or synthetic medium may be used as long as it contains carbon sources, nitrogen sources and inorganic salts assimilable by the microorganism and is capable of effective cultivation of the recombinant. As carbon sources, carbohydrates such as glucose, galactose, fructose, sucrose, raffinose, starch; organic acids such as acetic acid, propionic acid; and alcohols such as ethanol and propanol may be used. As nitrogen sources, ammonia; ammonium salts of inorganic or organic acids such as ammonium chloride, ammonium sulfate, ammonium acetate, ammonium phosphate; other nitrogen-containing compounds; Peptone; meat extract; corn steep liquor, various amino acids, etc. may be used. As inorganic substances, potassium dihydrogen phosphate, dipotassium hydrogen phosphate, magnesium phosphate, magnesium sulfate, sodium chloride, iron(II) sulfate, manganese sulfate, copper sulfate, calcium carbonate and the like may be used. Usually, the recombinant is subjected to shaking culture or aeration agitation culture under aerobic conditions at 26 to 36° C. Preferably, when the host is S. cerevisiae, the recombinant is cultured at 30° C. for 2 to 7 days. When the host is E. coli, the recombinant is cultured at 37° C. for 12 to 18 hours. The adjustment of pH is carried out using an inorganic or organic acid, an alkali solution or the like. During the cultivation, antibiotics such as ampicillin, chloramphenicol or aureobasidin A may be added to the medium if necessary.

When a recombinant incorporating an expression vector containing an inducible transcription promoter is cultured, an inducer may be added to the medium if necessary. For example, when GAL1 promoter was used, galactose may be used as a carbon source. When a microorganism (e.g., E. coli) transformed with an expression vector containing a promoter that is inducible by isopropyl-β-D-thiogalactopyranoside (IPTG) is cultured, IPTG may be added to the medium.

When cultured under the above-described conditions, the recombinant of the invention can produce prenyl alcohol(s) at high yield(s). In particular, when the host is AURGG101 and the vector is pYHMG044, the recombinant can produce 32 mg or more of prenyl alcohols per liter of the medium. It can produce even 150 mg/L or more depending on the culture conditions.

In the present invention, it is possible to increase the production efficiency of prenyl alcohols by adding to the above-described medium such substances as terpenoids, oils, or surfactants. Specific examples of these additives include the following.

Terpenoids: squalene, tocopherol, IPP, DMAPP

Oils: soybean oil, fish oil, almond oil, olive oil

Surfactants: Tergitol, Triton X-305, Span 85, Adekanol LG109(Asahi Denka), Adekanol LG294 (Asahi Denka), Adekanol LG295S (Asahi Denka), Adekanol LG297 (Asahi Denka), Adekanol B-3009A (Asahi Denka), Adekapluronic L-61 (Asahi Denka).

The concentrations of oils are 0.01% or more, preferably 1-3%. The concentrations of surfactants are 0.005-1%, preferably 0.05-0.5%. The concentrations of terpenoids are 0.01% or more, preferably 1-3%.

After the cultivation, the prenyl alcohol of interest is recovered by disrupting the microorganisms or cells by, e.g., homogenizing, when the alcohol(s) is produced within the microorganisms or cells. Alternatively, the alcohol(s) may be extracted directly using organic solvents without disrupting the cells. When the prenyl alcohol(s) of the invention is produced outside the microorganisms or cells, the culture broth is used as it is or subjected to centrifugation or the like to remove the microorganisms or cells. Thereafter, the prenyl alcohol(s) of interest is extracted from the culture by, e.g., extraction with an organic solvent. If necessary, the alcohol(s) may be further isolated and purified by various types of chromatography or the like.

In the present invention, preferable combinations of host strains and vectors as recombinant DNAs, as well as relationships between these combinations and yields of prenyl alcohols are as illustrated in Table 2 below.

TABLE 2

| Promoter | Gene | Host | | FOH Yield (mg/L) | NOH Yield (mg/L) |
|---|---|---|---|---|---|
| GAP | HMG1 | S. cerevisiae | A451 | 0.05, 0.65-11.2, 4.9-11.2 | 0.05, 0.05-0.16, 0.16 |
| GAP | HMG1 | S. cerevisiae | EUG8(from A451) | 0.2, 0.20-1.8, 1.8 | —, —, — |
| ADH, GAP, | HMG1 | S. cerevisiae | YPH499 | 0.05, 0.05-0.11, 0.11 | —, —, — |
| PGK, TEF | | | | | |
| GAP | HMG1 | S. cerevisiae | EUG12(from YPH499) | 5.9, 5.9-18.3, 18.3 | 0.13, 0.13-0.30, 0.30 |
| PGK, TEF | HMG1 | S. cerevisiae | YPH500 | —, —, — | —, —, — |
| GAP | HMG1 | S. cerevisiae | EUG27(from YPH500) | 3.2, 3.2-13.6, 13.6 | 0.05, 0.05-0.22, 0.22 |
| GAP | HMG1 | S. cerevisiae | W303-1A | —, —, — | —, —, — |
| GAP | HMG1 | S. cerevisiae | W303-1B | —, —, — | —, —, — |
| GAL | HMG1′ | S. cerevisiae | A451 | —, —, — | 0.05, —, — |
| GAL | HMG1′ | S. cerevisiae | AURGG101(from A451) | 0.05, 0.29-8.2, 8.2 | 0.05, 0.095-2.7, 2.7 |
| GAL | HMG1′ | S. cerevisiae | YPH499 | 0.05, 0.05-0.057, 0.057 | —, —, — |
| GAL | HMG1′ | S. cerevisiae | YPH500 | —, —, — | —, —, — |
| GAL | HMG1′ | S. cerevisiae | W303-1A | —, —, — | 0.05, 0.10-0.15, 0.15 |
| GAL | HMG1′ | S. cerevisiae | W303-1B | —, —, — | 0.05, 0.091-0.14, 0.14 |
| GAL | HMG04y | S. cerevisiae | A451 | 0.05, 0.22-0.51, 0.51 | 0.05, 0.05-0.058, 0.058 |
| GAL | HMG04y | S. cerevisiae | AURGG101(from A451) | 0.05, 0.05-158, 53-158 | 0.05, 0.05-23, 2.4-23 |
| GAL | HMG04y | S. cerevisiae | YPH499 | —, —, — | —, —, — |
| GAL | HMG04y | S. cerevisiae | YPH500 | —, —, — | —, —, — |
| GAL | HMG04y | S. cerevisiae | W303-1A | —, —, — | —, —, — |
| GAL | HMG04y | S. cerevisiae | W303-1B | —, —, — | —, —, — |
| GAL | HMGxxy | S. cerevisiae | A451 | 0.05, 0.05-0.21, 0.21 | 0.05, 0.05-0.12, 0.12 |
| GAL | HMGxxy | S. cerevisiae | AURGG101(from A451) | 0.05, 0.05-0.13, 0.13 | 0.05, 0.05-0.11, 0.11 |
| GAL | HMGxxy | S. cerevisiae | YPH499 | —, —, — | —, —, — |
| GAL | HMGxxy | S. cerevisiae | YPH500 | —, —, — | —, —, — |
| GAL | HMGxxy | S. cerevisiae | W303-1A | —, —, — | —, —, — |
| GAL | HMGxxy | S. cerevisiae | W303-1B | —, —, — | —, —, — |
| GAP&GAL | HMG&HMG04 | S. cerevisiae | AURGG101(from A451) | 22, 22-66, 66 | 12, 12-28, 28 |
| | ispA | E. coli | JM109 | 11, 11-93, 73-93 | —, —, — |
| | fps | E. coli | JM109 | 12, —, — | —, —, — |
| | ispA & idi | E. coli | JM109 | 0.15, 0.15-0.16, — | —, —, — |

In the columns of FOH yield and NOH yield, lower limit, preferable range and more preferable range are shown in this order from the left side.
Mark “—” means no data.

From Table 2, the following yields can be presented, for example.

(1) When a DNA comprising HMG1 or a mutant thereof (e.g., HMG1′) or a deletion mutant of this mutant (HMGxxy) ligated downstream of a constitutive promoter had been introduced into S. cerevisiae cells, the cells produced FOH at least at 0.05 mg/L, preferably at 0.05-18.3 mg/L, and produced NOH at least at 0.05 mg/L, preferably at 0.05-0.3 mg/L.

(2) When a DNA comprising HMG1 or a mutant thereof (e.g., HMG1′) or a deletion mutant of this mutant (HMGxxy) ligated downstream of an inducible promoter had been introduced into S. cerevisiae cells, the cells produced FOH at least at 0.05 mg/L, preferably at 0.05-158 mg/L, more preferably at 53-158 mg/L, and produced NOH at least at 0.05 mg/L, preferably at 0.05-23 mg/L, more preferably at 2.4-23 mg/L.

(3) When two DNAs comprising HMG1 ligated downstream of a constitutive promoter and HMG04y (a deletion mutant of HMG1′) ligated downstream of an inducible promoter, respectively, had been introduced into S. cerevisiae cells, the cells produced FOH at least at 22 mg/L, preferably at 22-66 mg/L, and produced NOH at least at 12 mg/L, preferably at 12-28 mg/L.

(4) When a DNA comprising HMG1 or a mutant thereof (e.g., HMG1′) or a deletion mutant of this mutant (HMGxxy) had been introduced into S. cerevisiae A451 cells or A451-derived cells, the cells produced FOH at least at 0.05 mg/L, preferably at 0.05-158 mg/L, more preferably at 53-158 mg/L, and produced NOH at least at 0.05 mg/L, preferably at 0.05-23 mg/L, more preferably at 2.4-23 mg/L.

(5) When a DNA comprising HMG1 or a mutant thereof (e.g., HMG1′) or a deletion mutant of this mutant (HMGxxy) had been introduced into S. cerevisiae YPH499 cells or YPH499-derived cells, the cells produced FOH at least at 0.05 mg/L, preferably at 0.05-18.3 mg/L, more preferably at 5.9-18.3 mg/L, and produced NOH at least at 0.05 mg/L, preferably at 0.13-0.30 mg/L.

(6) When a DNA comprising HMG1 or a mutant thereof (e.g., HMG1′) or a deletion mutant of this mutant (HMGxxy) had been introduced into S. cerevisiae YPH500 cells or YPH500-derived cells, the cells produced FOH at least at 3.2 mg/L, preferably at 3.2-13.6 mg/L, and produced NOH at least at 0.05 mg/L, preferably at 0.05-0.22 mg/L.

(7) When a DNA comprising HMG1 or a mutant thereof (e.g., HMG1′) had been introduced into S. cerevisiae cells, the cells produced FOH at least at 0.05 mg/L, preferably at 0.05-18.3 mg/L, and produced NOH at least at 0.05 mg/L, preferably at 0.05-2.7 mg/L.

(8) When a DNA comprising HMGxxy (a deletion mutant of HMG1′) had been introduced into S. cerevisiae cells, the cells produced FOH at least at 0.05 mg/L, preferably at 0.05-158 mg/L, more preferably at 53-158 mg/L, and produced NOH at least at 0.05 mg/L, preferably at 0.05-23 mg/L, more preferably at 2.4-23 mg/L.

(9) A plasmid comprising a substitution mutant of E. coli FPP synthase gene ispA was introduced into E. coli. When the resultant cells were cultured in a liquid medium containing IPP and DMAPP and then treated with phosphatase, the cells produced FOH at least at 11 mg/L, preferably at 11-90 mg/L, more preferably at 64-90 mg/L.

(10) When ispA and idi had been introduced into E. coli, the cells produced FOH at least at 0.15 mg/L, preferably at 0.15-0.16 mg/L, as a result of phosphatase treatment even without the addition of IPP and DMAPP.

### BRIEF DESCRIPTION OF THE DRAWINGS

FIG. 1 is a diagram showing a metabolic pathway in which mevalonate pathway-related enzymes are involved.

FIG. 2A is a diagram showing construction of deletion mutants of HMG1 gene.

FIG. 2B shows patterns of substitution mutations.

FIG. 3 is a diagram showing plasmid pRS414.

FIG. 4 is a diagram showing plasmid pYES2.

FIG. 5 is a diagram showing sequences for ADH1 promoter and terminator.

FIG. 6A is a diagram showing plasmid pRS414PTadh.

FIG. 6B is a diagram showing plasmid pRS414TPadh.

FIG. 7A-1 is a diagram showing plasmid pRS434ADH.

FIG. 7A-2 is a diagram showing plasmid pRS434GAP.

FIG. 7B-1 is a diagram showing plasmid pRS434PGK.

FIG. 7B-2 is a diagram showing plasmid pRS434TEF.

FIG. 7C-1 is a diagram showing plasmid pRS436ADH.

FIG. 7C-2 is a diagram showing plasmid pRS436GAP.

FIG. 7D-1 is a diagram showing plasmid pRS436PGK.

FIG. 7D-2 is a diagram showing plasmid pRS436TEF.

FIG. 7E-1 is a diagram showing plasmid pRS444ADH.

FIG. 7E-2 is a diagram showing plasmid pRS444GAP.

FIG. 7F-1 is a diagrams showing plasmid pRS444PGK.

FIG. 7F-2 is a diagram showing plasmid pRS444TEF.

FIG. 7G-1 is a diagram showing plasmid pRS446ADH.

FIG. 7G-2 is a diagram showing plasmid pRS446GAP.

FIG. 7H-1 is a diagram showing plasmid pRS446PGK.

FIG. 7H-2 is a diagram showing plasmid pRS446TEF.

FIG. 8 is a physiological map of plasmid pALHMG106.

FIG. 9 presents photographs showing the results of Southern blotting.

FIG. 10 presents photographs showing the results of PCR mapping.

FIG. 11 presents photographs showing the results of Northern blotting.

FIG. 12A presents graphs showing the specific activity of each prenyl-diphosphate synthase in a crude enzyme solution.

FIG. 12B presents graphs showing the specific activity of each prenyl-diphosphate synthase in a crude enzyme solution.

FIG. 13 is a graph showing prenyl alcohol yields when pRS434GAP-HMG1 or pRS444GAP-HMG1 has been transferred into A451 strain.

FIG. 14 is a graph showing prenyl alcohol yields when pRS434GAP-HMG1 or pRS444GAP-HMG1 has been transferred into A451 strain.

FIG. 15 presents graphs showing prenyl alcohol yields when pRS434GAP-HMG1 or pRS444GAP-HMG1 has been transferred into A451 strain.

FIG. 16 presents graphs showing prenyl alcohol yields when pRS414PTadh-HMG1, pRS414TPadh-HMG1, pRS434GAP-HMG1, pRS444GAP-HMG1, pRS434PGK-HMG1, pRS444PGK-HMG1, pRS434TEF-HMG1 or pRS444TEF-HMG1 has been transferred into YPH499 strain.

FIG. 17 presents graphs showing prenyl alcohol yields when pRS434GAP-HMG1 or pRS444GAP-HMG1 has been transferred into EUG8 strain.

FIG. 18 presents graphs showing prenyl alcohol yields when pRS434GAP-HMG1 or pRS444GAP-HMG1 has been transferred into EUG12 strain.

FIG. 19 presents graphs showing prenyl alcohol yields when pRS434GAP-HMG1 or pRS444GAP-HMG1 has been transferred into EUG27 strain.

FIG. 20A presents graphs showing prenyl alcohol yields when pYES-HMG1 or pYHMG044 has been transferred into A451 strain.

FIG. 20B presents graphs showing prenyl alcohol yields when pYES-HMG1 or pYHMG044 has been transferred into AURGG101 strain.

FIG. 21 presents graphs showing prenyl alcohol yields when pYES-HMG1 has been transferred into W303-1A or W303-1B.

FIG. 22 presents graphs showing prenyl alcohol yields when pYHMG026, pYHMG044, pYHMG056, pYHMG062, pYHMG076, pYHMG081, pYHMG100, pYHMG112 or pYHMG122 has been transferred into A451 strain.

FIG. 23 presents graphs showing prenyl alcohol yields when pYHMG026, pYHMG044, pYHMG056, pYHMG062, pYHMG076, pYHMG081, pYHMG100, pYHMG112 or pYHMG122 has been transferred into AURGG101 strain.

FIG. 24 presents graphs showing prenyl alcohol yields when pYHMG026, pYHMG044, pYHMG056, pYHMG062, pYHMG076, pYHMG081, pYHMG100, pYHMG112 or pYHMG122 has been transferred into AURGG101 strain (the graphs in FIG. 23 are enlarged).

FIG. 25 is a graph showing prenyl alcohol yields when pRS434GAP-HMG1 or pRS444GAP-HMG1 has been introduced into AURGG101 strain together with pYHMG044.

FIG. 26 is a graph showing prenyl alcohol yields when a mutant ispA gene-transferred E. coli was cultured in a liquid medium containing IPP and DMAPP.

FIG. 27 is a graph showing prenyl alcohol yields when a mutant ispA gene-transferred E. coli was cultured in a liquid medium without IPP and DMAPP.

FIG. 28 is a graph showing prenyl alcohol yields and cell counts when a recombinant 15-2 clone (pYHMG044/AURGG101) was cultured in ajar fermenter.

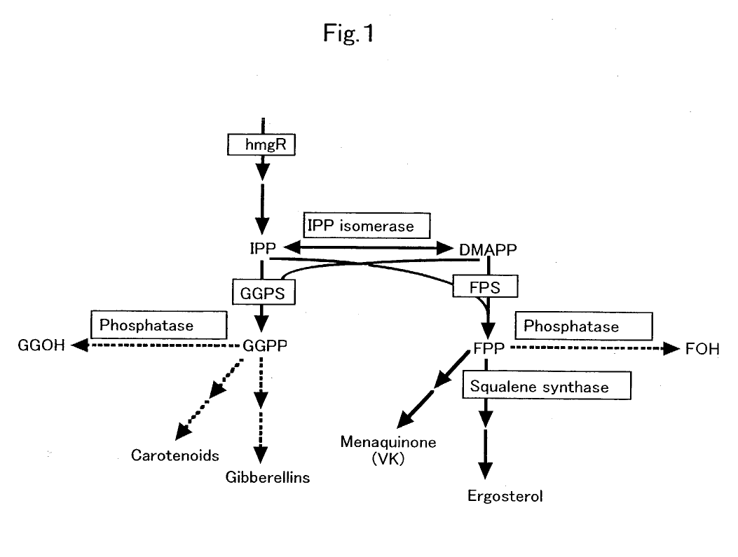

### BEST MODE FOR CARRYING OUT THE INVENTION

Hereinbelow, the present invention will be described more specifically with reference to the following Examples. However, the technical scope of the present invention is not limited to these Examples.

### EXAMPLE 1

### Construction of Expression Vectors

Vectors were constructed using E. coli SURE2 supercompetent cells purchased from Stratagene (La Jolla, Calif.) as a host. For the preparation of genomic DNA from S. cerevisiae and for testing the introduction of resultant vectors, YPH499 strain (Stratagene) was used.

(1) E. coli-S. cerevisiae Shuttle Vectors

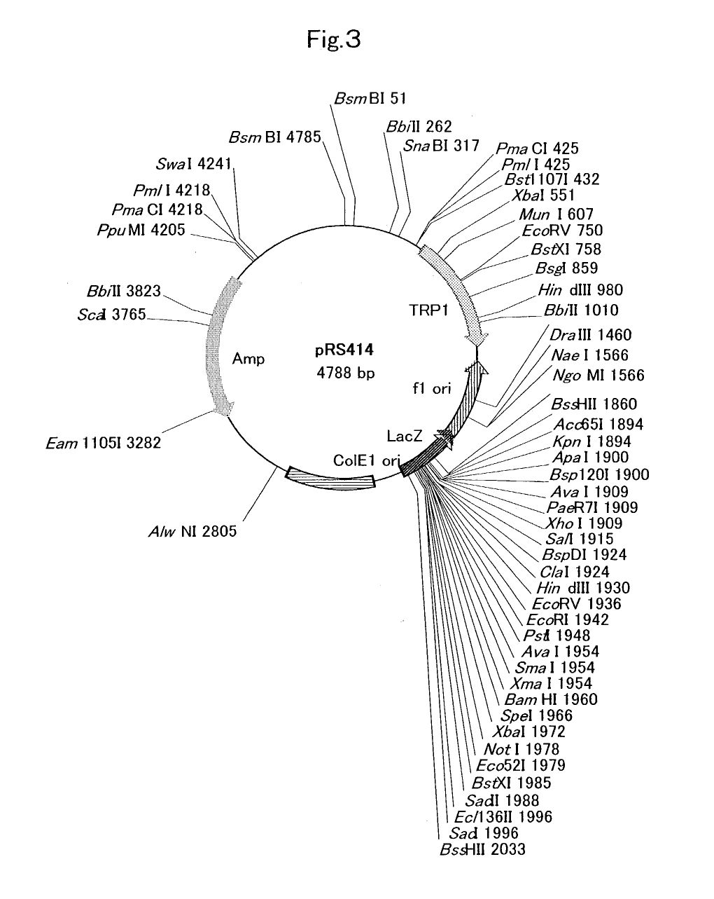

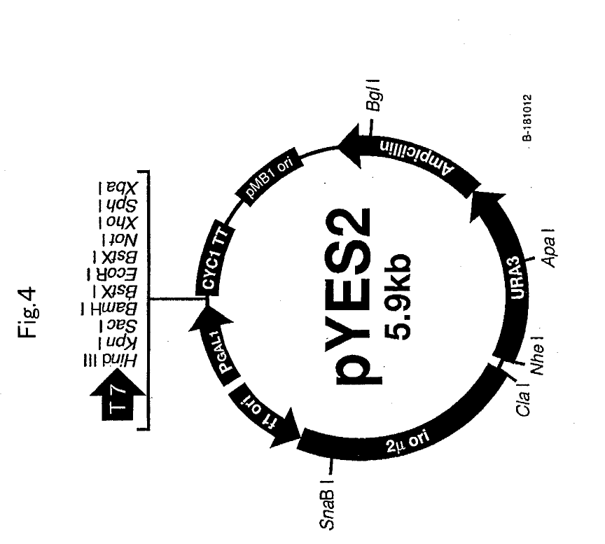

Plasmids pRS404 and pRS414 (FIG. 3) were purchased from Stratagene. Plasmid pAUR123 was purchased from Takara, and plasmid pYES2 (FIG. 4) was purchased from Invitrogen (Carlsbad, Calif.).

(2) Genomic DNA

Dr. GenTLE™, a genomic DNA preparation kit for yeast, was purchased from Takara. Genomic DNA was prepared from S. cerevisiae YPH499 according to the protocol attached to the kit.

(3) Insertion of ADH1p-ADH1t Fragment into pRS414

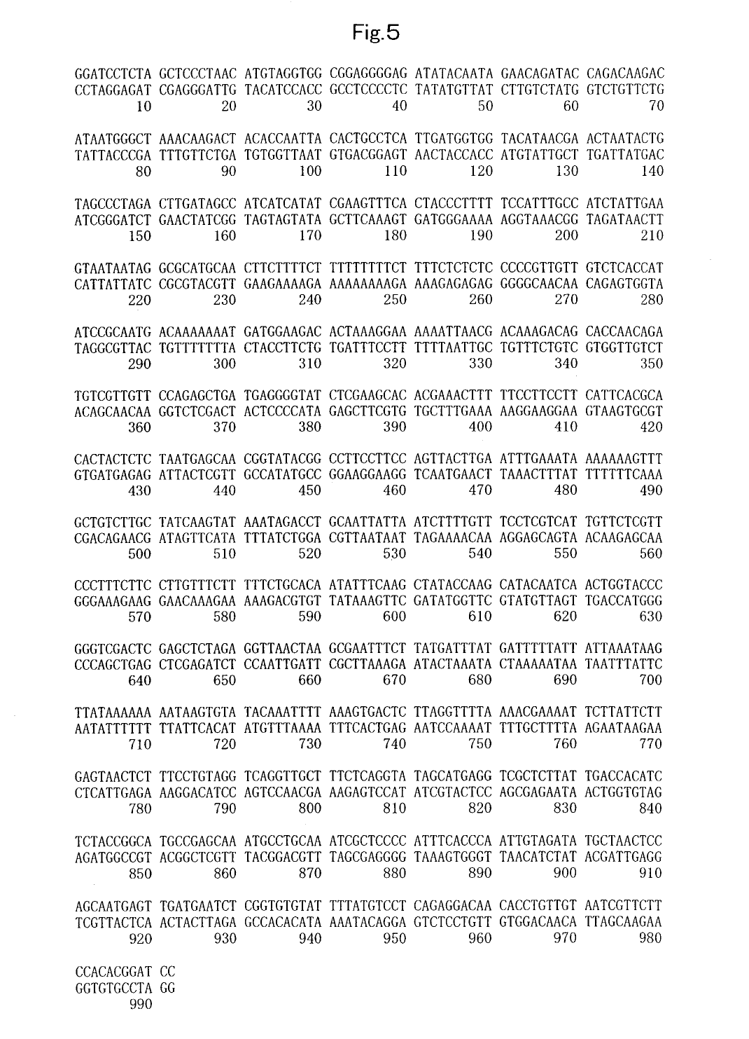

Plasmid pRS414 (FIG. 3) was digested with NaeI and PvuII to obtain a 4.1 kbp fragment without f1 ori and LacZ moieties. This fragment was purified by agarose gel electrophoresis. Plasmid pAUR123 was digested with BamHI and blunt-ended with Klenow enzyme. Then, a 1.0 kbp fragment containing ADH1 transcription promoter (ADH1p) and ADH1 transcription terminator (ADH1t) (FIG. 5; SEQ ID NO: 17) was purified by agarose gel electrophoresis. The 4.1 kbp fragment from pRS414 still retained the replication origins for E. coli and yeast, a transformation marker Amp^{r} for E. coli, and an auxotrophic marker TRP1 for yeast. On the other hand, the 1.0 kbp fragment from pAUR123 contained ADH1p, ADH1t, and a cloning site flanked by them. These two fragments were ligated to each other with a DNA ligation kit (Takara) and transformed into SURE2 cells.

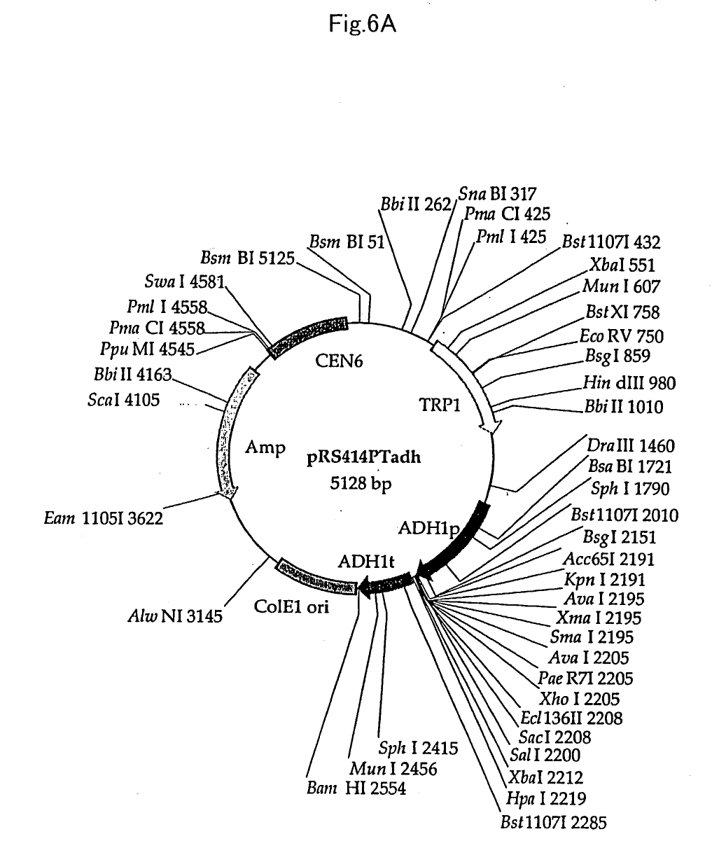

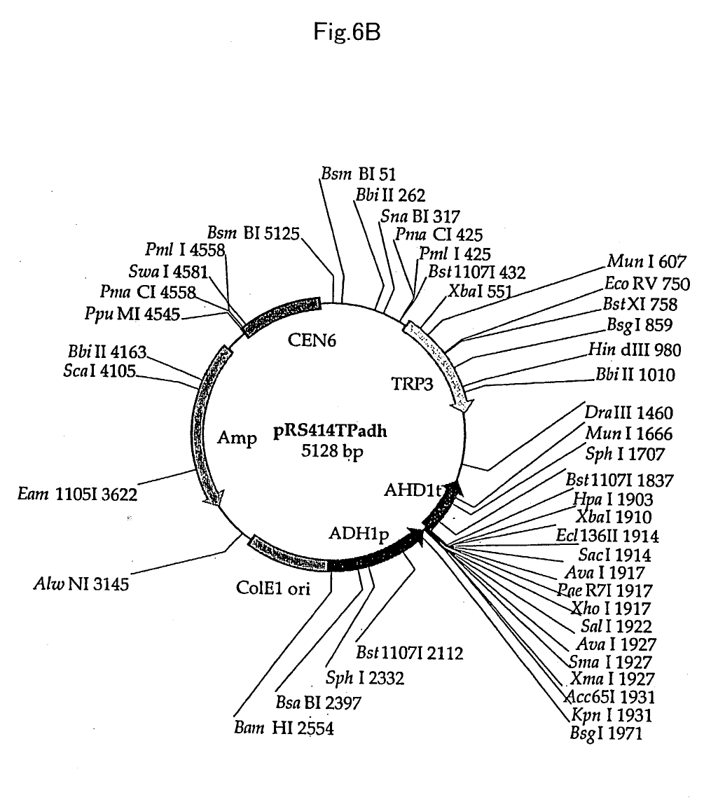

Plasmid DNA was prepared from the resultant recombinant. Mapping of the DNA with SalI and ScaI revealed that the ADH1p-ADHt fragment has been inserted into pRS414 in opposite directions to thereby yield two plasmids pRS414PTadh and pRS414TPadh (FIG. 6).

(4) Insertion of CYC1t Fragment into pRS Vectors

CYC1t (CYC1 transcription terminator) fragment was prepared by PCR. The following oligo-DNAs, XhoI-Tcyc1FW and ApaI-Tcyc1RV, were used as PCR primers. As a template, pYES2 was used.

| | | |
|---|---|---|
| XhoI-Tcyc1FW: | 5′- TGC ATC TCG AGG GCC GCA TCA TGT AAT TAG -3′ | (SEQ ID NO: 18) |
| ApaI-Tcyc1RV: | 5′- CAT TAG GGC CCG GCC GCA AAT TAA AGC CTT CG -3′ | (SEQ ID NO: 19) |

Briefly, 50 μl of a reaction solution containing 0.1 μg of pYES2, 50 pmol of each primer DNA, 1× Pfu buffer containing MgSO₄ (Promega, Madison, Wisc.), 10 nmol dNTPs, 1.5 units of Pfu DNA polymerase (Promega) and 1 μl of Perfect Match polymerase enhancer (Stratagene) was prepared. The reaction conditions were as follows: first denaturation at 95° C. for 2 min; 30 cycles of denaturation at 95° C. for 45 sec, annealing at 60° C. for 30 sec, and extension at 72° C. for 1 min; and final extension at 72° C. for 5 min. After completion of the reaction, the solution was stored at 4° C. The amplified DNA was digested with XhoI and ApaI, and the resultant 260 bp DNA fragment was purified by agarose gel electrophoresis to obtain CYC1t-XA.

CYC1t-XA was inserted into the XhoI-ApaI site of pRS404 and pRS406 to thereby obtain pRS404Tcyc and pRS406Tcyc, respectively.

(5) Preparation of Transcription Promoters

DNA fragments comprising transcription promoters were prepared by PCR using pAUR123 or yeast genomic DNA as a template. The DNA primers used are as follows.

| | | |
|---|---|---|
| SacI-Padh1FW: | 5′-GAT CGA GCT CCT CCC TAA CAT GTA GGT GGC GG-3′ | (SEQ ID NO: 20) |
| SacII-Padh1RV: | 5′-CCC GCC GCG GAG TTG ATT GTA TGC TTG GTA TAG C-3′ | (SEQ ID NO: 21) |
| SacI-Ptdh3FW: | 5′-CAC GGA GCT CCA GTT CGA GTT TAT CAT TAT CAA-3′ | (SEQ ID NO: 22) |
| SacII-Ptdh3RV: | 5′-CTC TCC GCG GTT TGT TTG TTT ATG TGT GTT TAT TC-3′ | (SEQ ID NO: 23) |
| SacI-PpgklFW: | 5′-TAG GGA GCT CCA AGA ATT ACT CGT GAG TAA GG-3′ | (SEQ ID NO: 24) |
| SacII-Ppgk1RV: | 5′-ATA ACC GCG GTG TTT TAT ATT TGT TGT AAA AAG TAG-3′ | (SEQ ID NO: 25) |
| SacI-Ptef2FW: | 5′-CCG CGA GCT CTT ACC CAT AAG GTT GTT TGT GAC G-3 | (SEQ ID NO: 26) |
| SacII-Ptef2RV: | 5′-CTT TCC GCG GGT TTA GTT AAT TAT AGT TCG TTG ACC-3′ | (SEQ ID NO: 27) |

For the amplification of ADH1 transcription promoter (ADH1p), SacI-Padh1FW and SacII-Padh1RV were used as PCR primers and pAUR123 as a template. For the amplification of TDH3 (GAP) transcription promoter (TDH3p (GAPp)), SacI-Ptdh3FW and SacII-Ptdh3RV were used as PCR primers; for the amplification of PGK1 transcription promoter (PGK1p), SacI-Ppgk1FW and SacII-Ppgk1RV were used as PCR primers; and for the amplification of TEF2 transcription promoter (TEF2p), SacI-Ptef2FW and SacII-Ptef2RV were used as PCR primers. For these promoters, yeast genomic DNA was used as a template. As a reaction solution, a 100 μl solution containing 0.1 μg of pAUR123 or 0.46 μg of yeast genomic DNA, 100 pmol of each primer DNA, 1×ExTaq buffer (Takara), 20 nmol dNTPs, 0.5 U of ExTaq DNA polymerase (Takara) and 1 μl of Perfect Match polymerase enhancer was prepared. The reaction conditions were as follows: first denaturation at 95° C. for 2 min; 30 cycles of denaturation at 95° C. for 45 sec, annealing at 60° C. for 1 min, and extension at 72° C. for 2 min; and final extension at 72° C. for 4 min. After completion of the reaction, the solution was stored at 4° C. The amplified 4 types of DNAs were digested with SacI and SacII, and the resultant 620 bp, 680 bp, 710 bp and 400 bp DNA fragments were purified separately by agarose gel electrophoresis to thereby obtain ADH1p, TDH3p, PGK1p and TEF2p, respectively.

(6) Preparation of 2μ DNA Replication Origin Site

pYES2, which is a YEp vector, was digested with SspI and NheI. The resultant 1.5 kbp fragment containing 2μ DNA replication origin (2μ ori) was purified by agarose gel electrophoresis and then blunt-ended. This DNA fragment was designated 2μOriSN.

(7) Preparation of YEp Type Expression Vectors

2μOriSN was inserted into the NaeI site of pRS404Tcyc and pRS406Tcyc pretreated with BAP (bacterial alkaline phosphatase: Takara). The resultant plasmids were transformed into E. coli SURE2, and then plasmid DNA was prepared. The plasmid DNA was digested with DraIII; and EcoRI, HpaI or PstI; and PvuII, followed by agarose gel electrophoresis to examine the insertion and the direction of 2μ ori. The resultant pRS404Tcyc and pRS406Tcyc into which 2μ ori had been inserted in the same direction as in pYES2 were designated pRS434Tcyc2μ Ori and pRS436Tcyc2μ Ori, respectively. The resultant pRS404Tcyc and pRS406Tcyc into which 2μ ori had been inserted in the opposite direction to that in pYES2 were designated pRS444Tcyc2μOri and pRS446Tcyc2μOri, respectively.

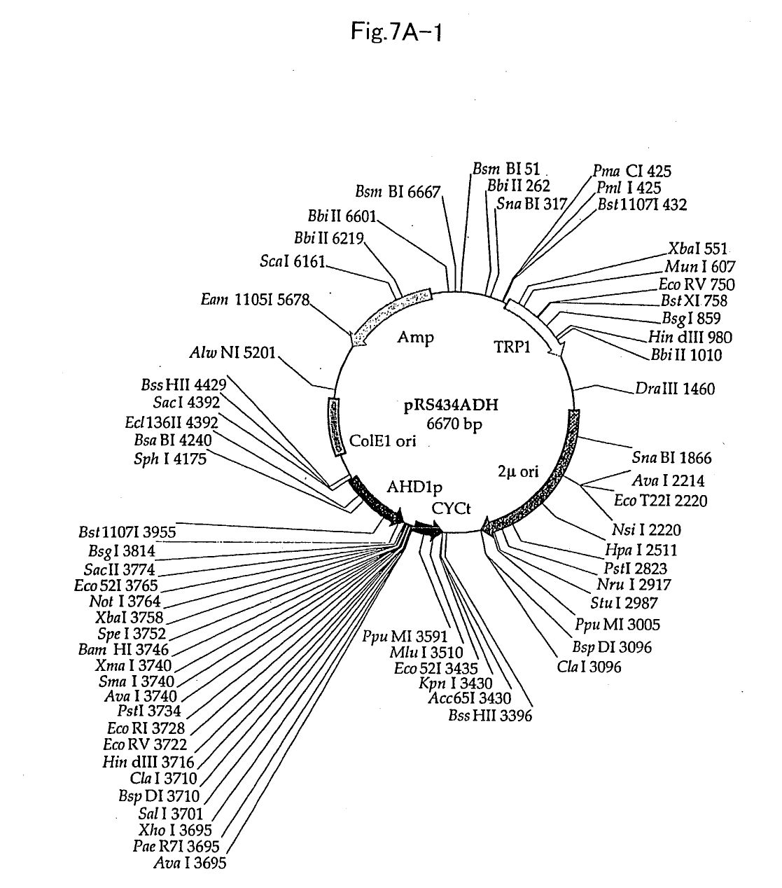

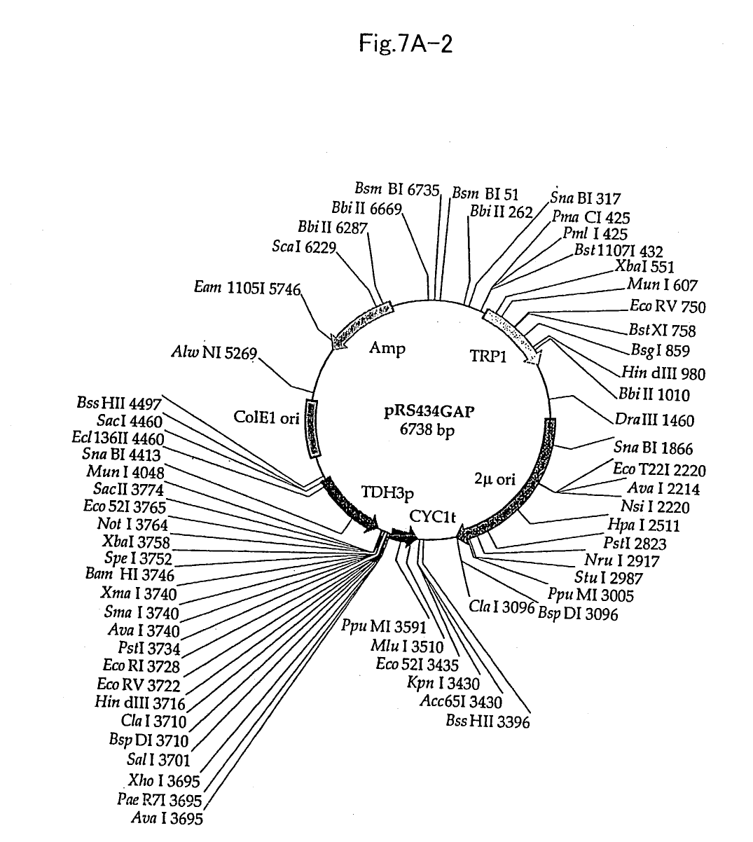

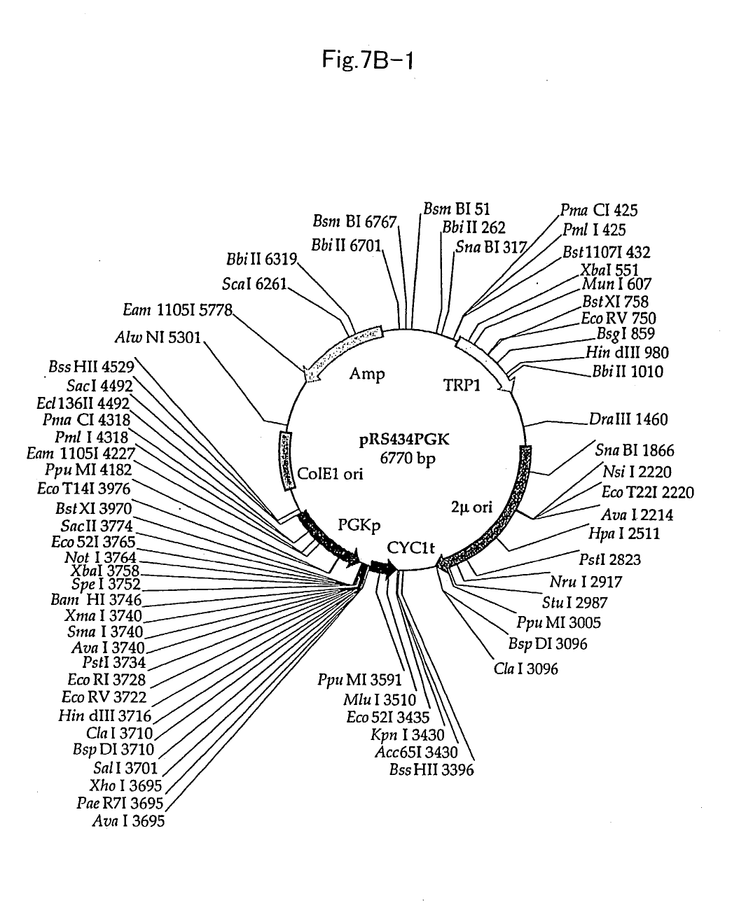

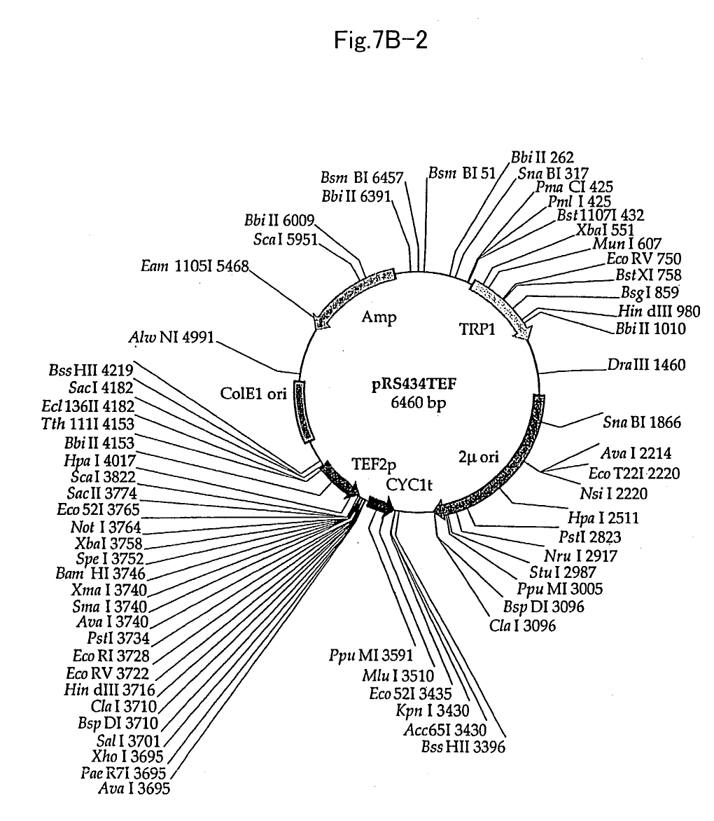

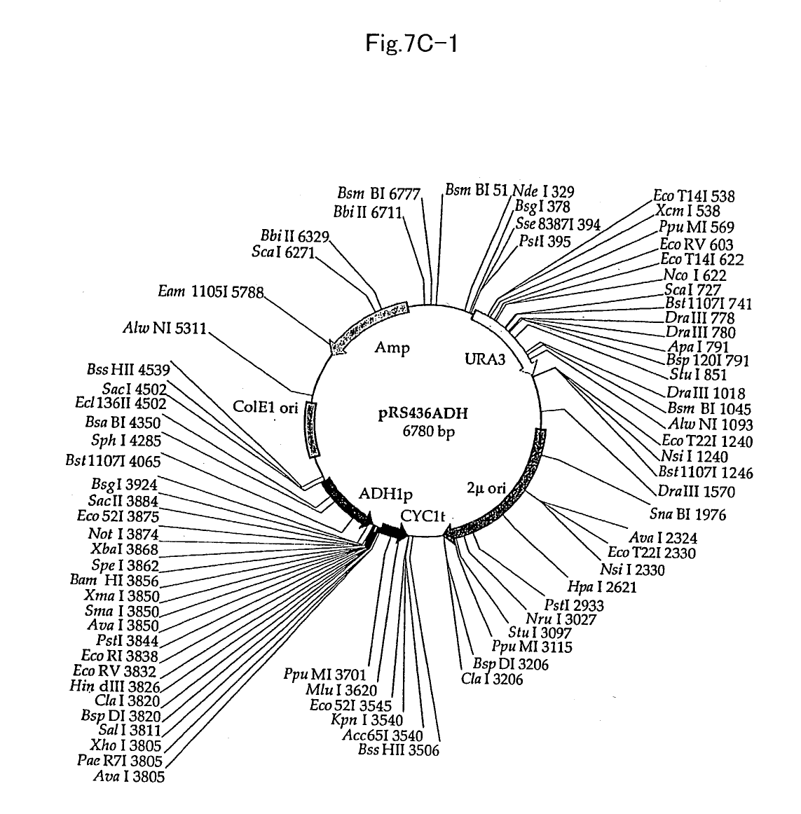

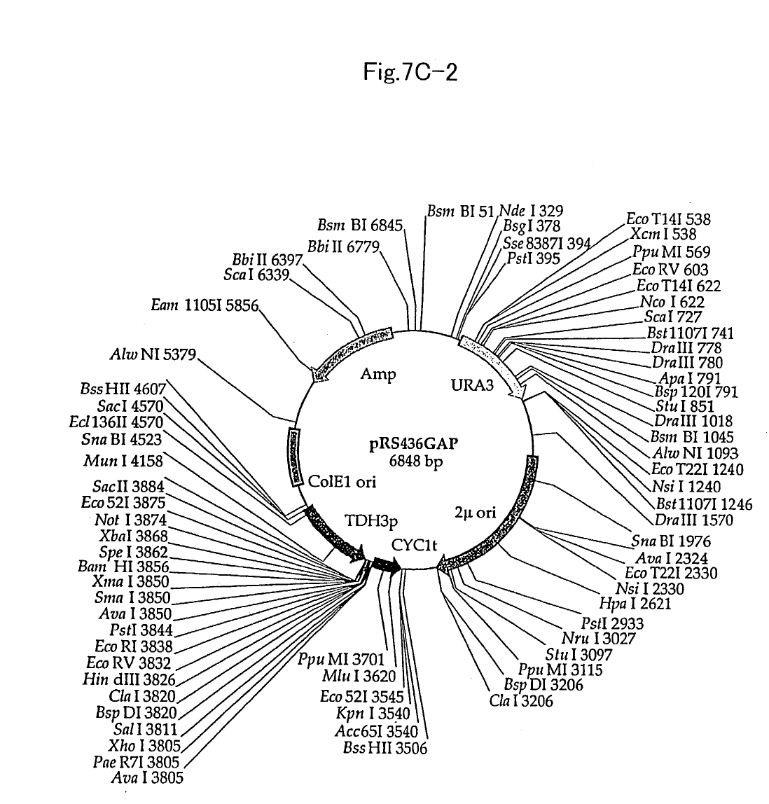

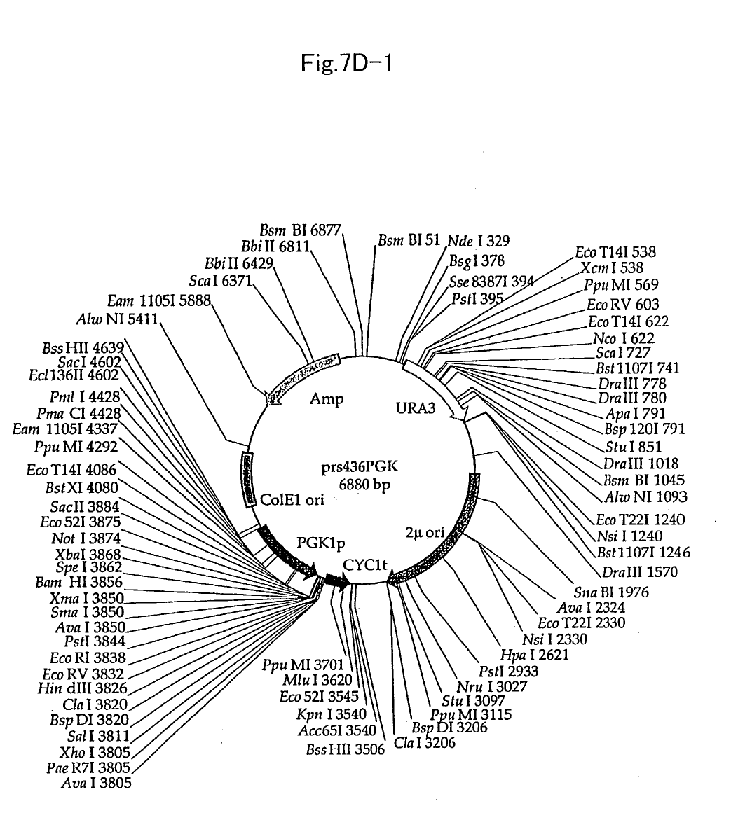

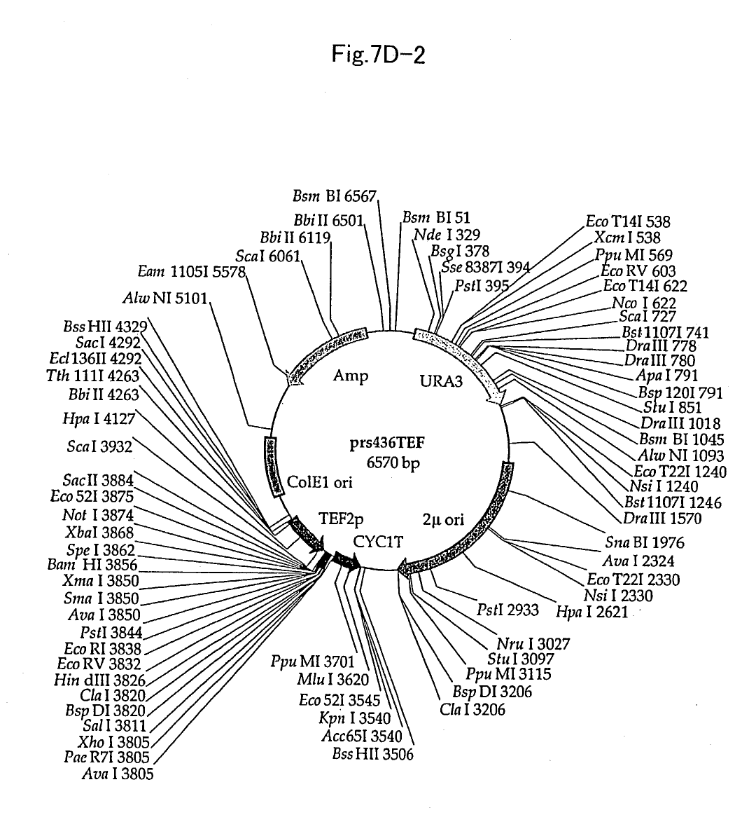

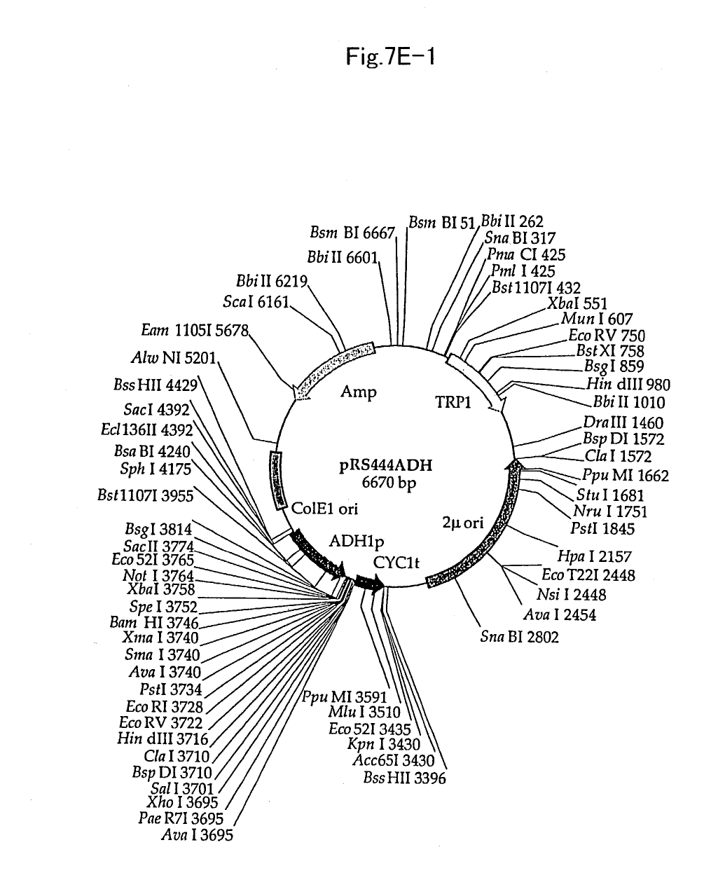

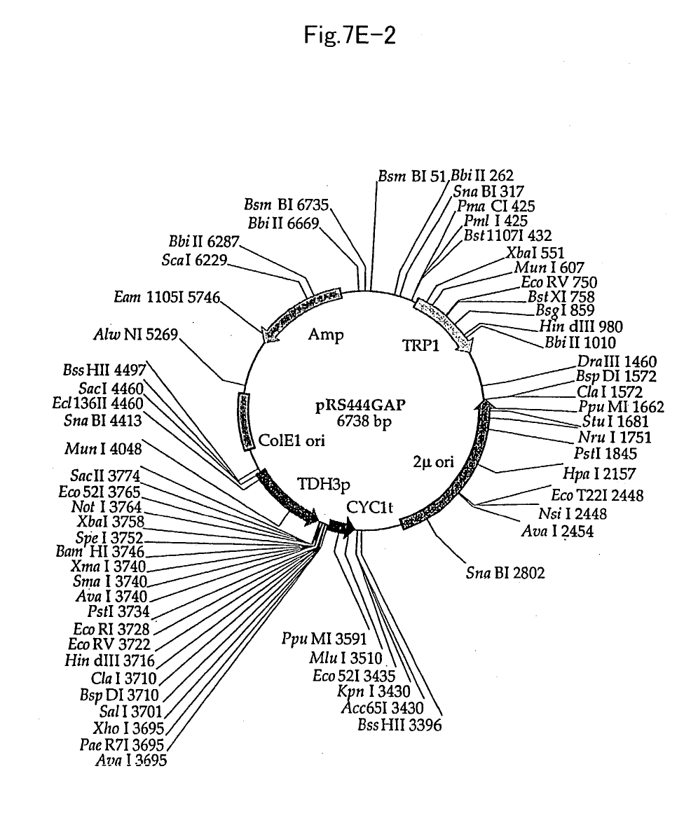

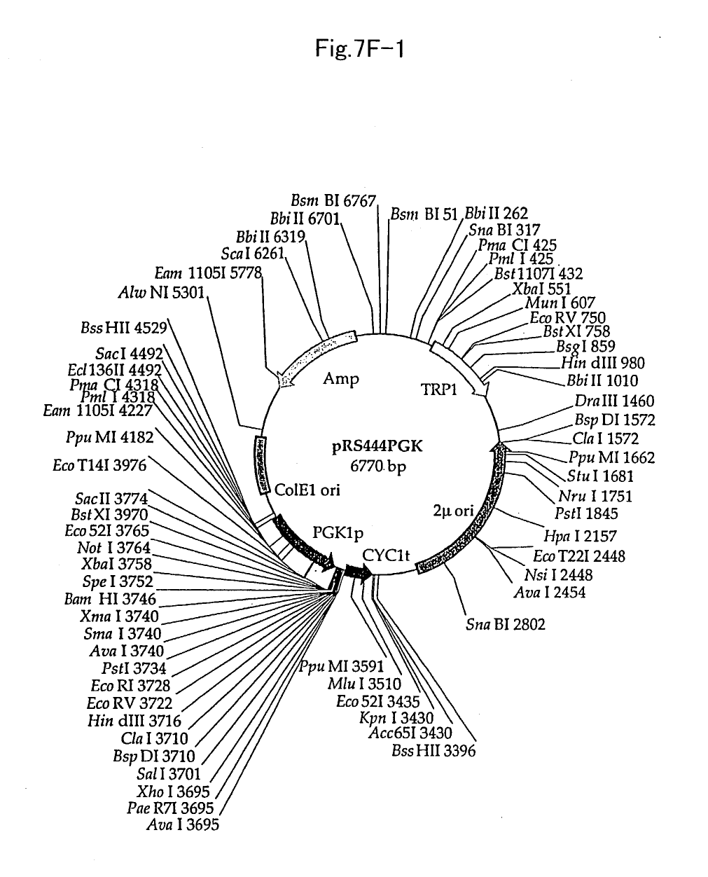

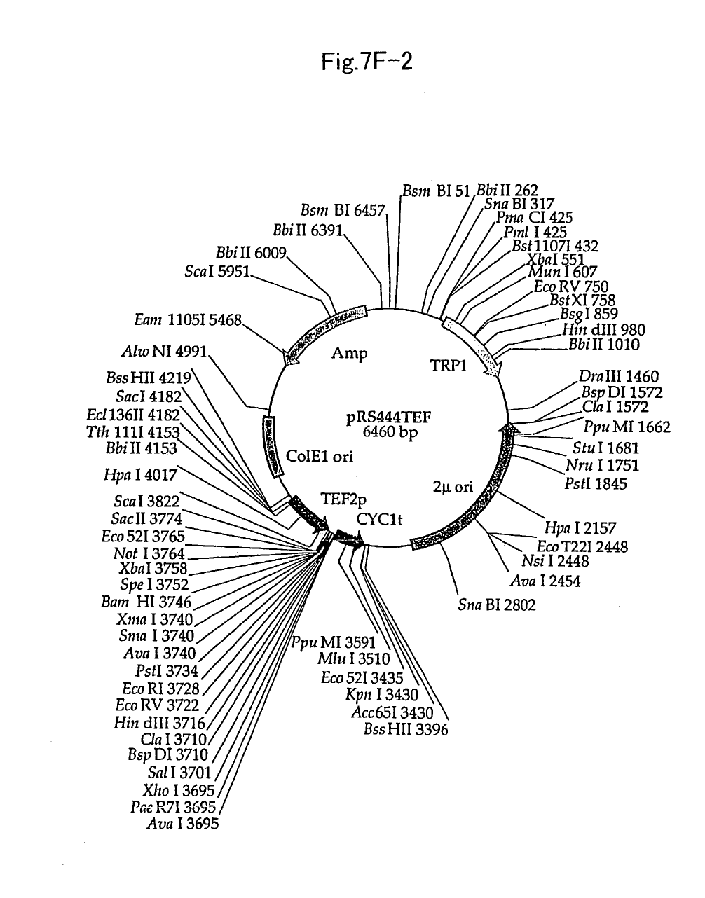

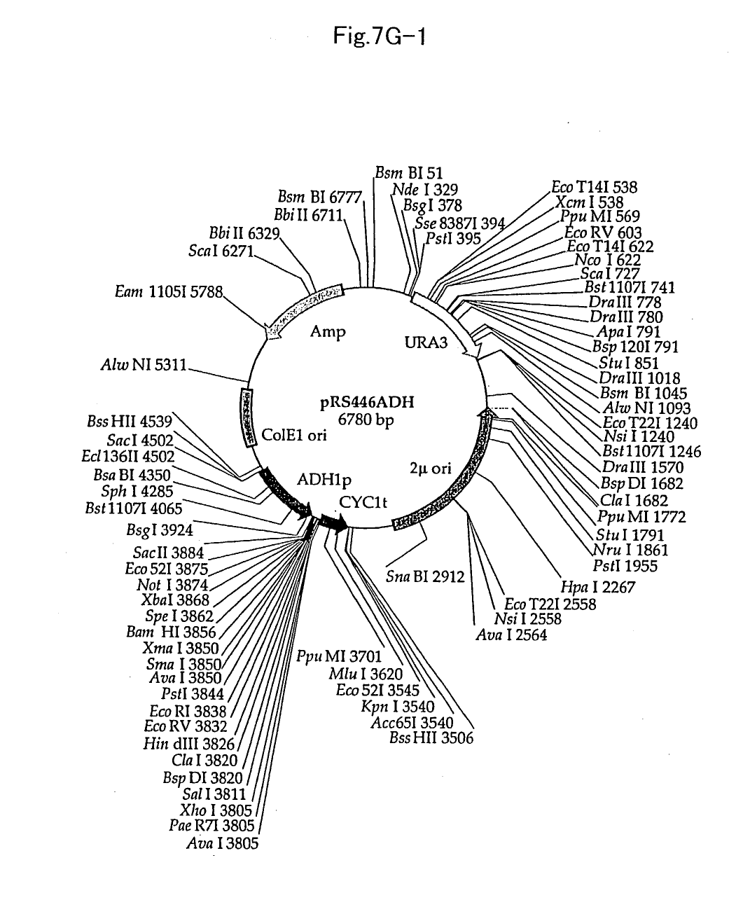

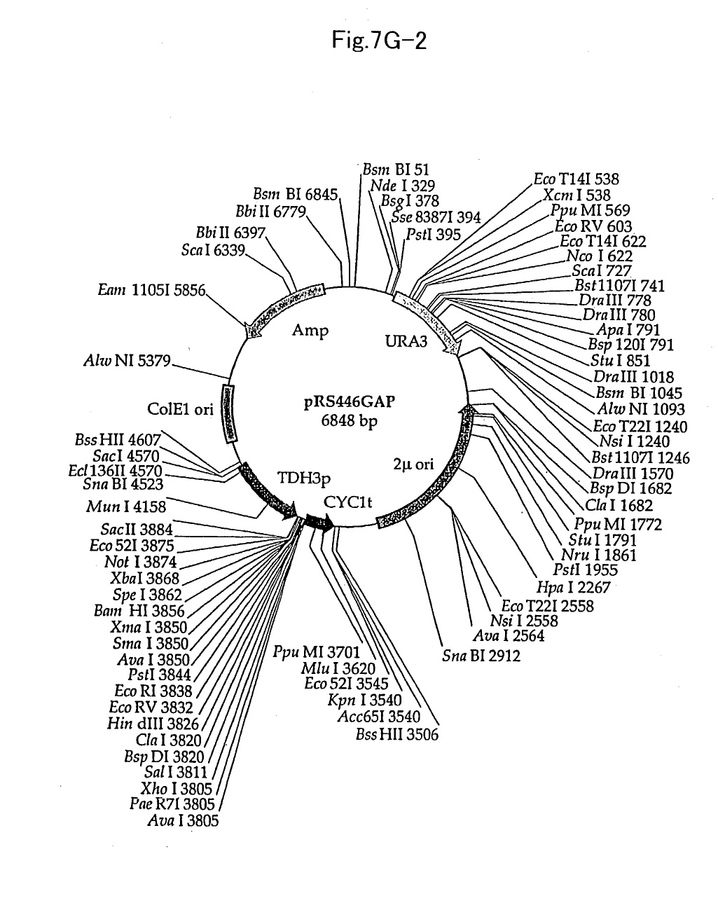

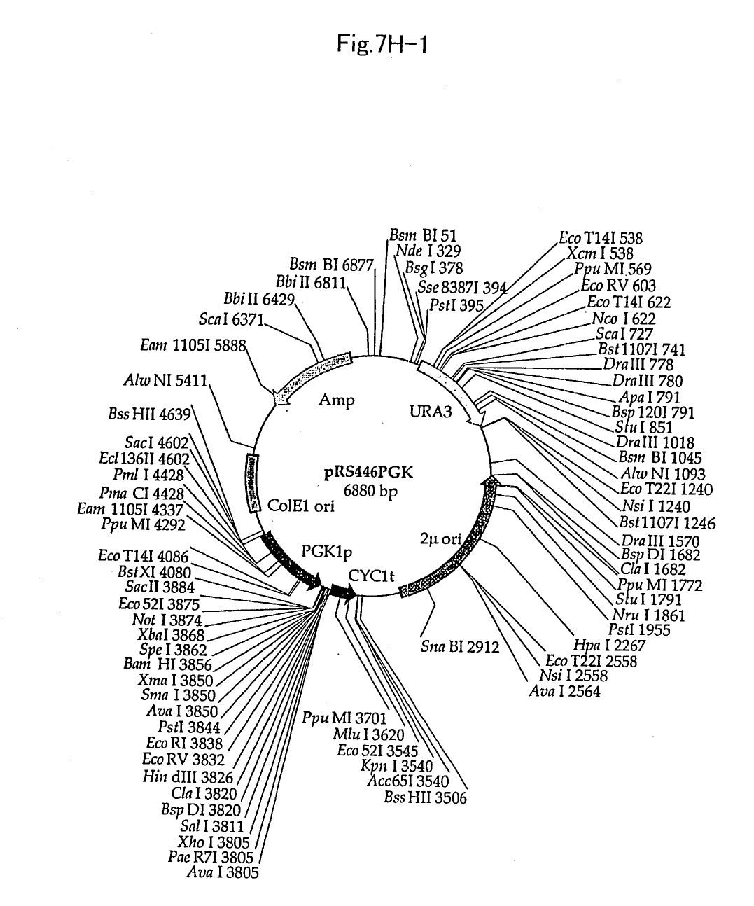

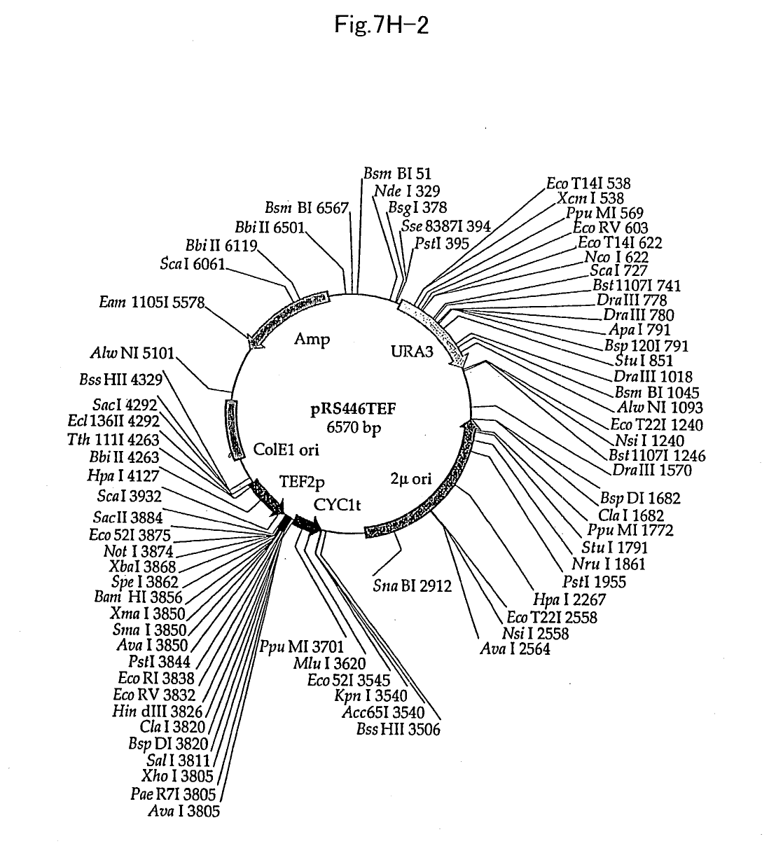

A transcription promoter-containing fragment, i.e., ADH1p, TDH3p (GAPp), PGK1p or TEF2p, was inserted into the SacI-SacII site of the above-described four plasmids pRS434Tcyc2μOri, pRS436Tcyc2μOri, pRS444Tcyc2μOri and pRS446Tcyc2μOri to clone the DNA. As a result, the following plasmids were obtained: (i) pRS434ADH, pRS434GAP, pRS434PGK and pRS434TEF from pRS434Tcyc2Ori; (ii) pRS436ADH, pRS436GAP, pRS436PGK and pRS436TEF from pRS436Tcyc2μOri; (iii) pRS⁴⁴⁴ADH, pRS444GAP, pRS444PGK and pRS444TEF from pRS444Tcyc2μOri; (iv) pRS446ADH, pRS446GAP, pRS446PGK and pRS446TEF from pRS446Tcyc2μOri (FIGS. 7A-7H).

The expression vectors prepared in the present invention are summarized in Table 3 below.

TABLE 3

| | | Marker and | | Promoter, Terminator and | | | | |
|---|---|---|---|---|---|---|---|---|
| Vector | Type | Direction* | | Direction* | | | ori and Direction* | |
| pRS414PTadh | YCp | TRP1 | + | ADH1 | ADH1 | + | ARS4 & CEN6 | + |
| pRS414TPadh | YCp | TRP1 | + | ADH1 | ADH1 | − | ARS4 & CEN6 | + |
| pRS434ADH | YEp | TRP1 | + | ADH1 | CYC1 | − | 2 μ | + |
| pRS434GAP | YEp | TRP1 | + | TDH3 | CYC1 | − | 2 μ | + |
| pRS434PGK | YEp | TRP1 | + | PGK1 | CYC1 | − | 2 μ | + |
| pRS434TEF | YEp | TRP1 | + | TEF2 | CYC1 | − | 2 μ | + |
| pRS436ADH | YEp | URA3 | + | ADH1 | CYC1 | − | 2 μ | + |
| pRS436GAP | YEp | URA3 | + | TDH3 | CYC1 | − | 2 μ | + |
| pRS436PGK | YEp | URA3 | + | PGK1 | CYC1 | − | 2 μ | + |
| pRS436TEF | YEp | URA3 | + | TEF2 | CYC1 | − | 2 μ | + |
| pRS444ADH | YEp | TRP1 | + | ADH1 | CYC1 | − | 2 μ | − |
| pRS444GAP | YEp | TRP1 | + | TDH3 | CYC1 | − | 2 μ | − |
| pRS444PGK | YEp | TRP1 | + | PGK1 | CYC1 | − | 2 μ | − |
| pRS444TEF | YEp | TRP1 | + | TEF2 | CYC1 | − | 2 μ | − |
| pRS446ADH | YEp | URA3 | + | ADH1 | CYC1 | − | 2 μ | − |
| pRS446GAP | YEp | URA3 | + | TDH3 | CYC1 | − | 2 μ | − |
| pRS446PGK | YEp | URA3 | + | PGK1 | CYC1 | − | 2 μ | − |
| pRS446TEF | YEp | URA3 | + | TEF2 | CYC1 | − | 2 μ | − |

*The + and − marks appearing after marker and gene expression transcription unit indicate downstream direction and upstream direction, respectively. The + mark appearing after ori indicates that ori is inserted in the same direction as in pRS (for YCp vectors) or pYES (for YEp vectors); the − mark indicates that ori is inserted in the direction opposite to that in pRS (for YCp vectors) or pYES (for YEp vectors).

(8) Introduction of YEp Type Expression Vectors into Yeast

In order to examine whether the DNA replication region of the prepared YEp type expression vectors functions or not, about 40 ng of each YEp type expression vector was introduced into YPH499 strain using Frozen-EZ Yeast Transformation II (Zymo Research, Orange, Calif.). (The procedures followed the protocol attached to the kit.) Then, colonies growing on SD-W (DOB+CMS (−Trp); BIO101, Vista, Calif.) agar plate at 30° C. were examined. The results are shown in Table 4 below.

TABLE 4

| | | ADH | GAP | PGK | TEF |
|---|---|---|---|---|---|
| pRS | 434 | >1000 | >1000 | >1000 | >1000 |
| | 436 | 500 | >1000 | >1000 | 300 |
| | 444 | >1000 | >1000 | >1000 | >1000 |
| | 446 | 250 | >1000 | >1000 | 100 |

The results shown in Table 4 revealed that each of the YEp type vectors prepared in the invention functions normally as a vector.

### EXAMPLE 2

### Cloning of Genes

(1) Cloning of HMG-CoA Reductase Gene (HMG1′ Gene) by PCR

The cloning of S. cerevisiae HMG1′ gene was carried out as described below.

Based on information on S. cerevisiae-derived HMG1 gene (Accession No. M22002) (M. E. Basson, et al., Mol. Cell. Biol. 8, 3797-3808 (1988): SEQ ID NO: 1) registered in the GenBank, a pair of primers were designed which are specific to those nucleotide sequences corresponding to an N-terminal and a C-terminal region of the protein encoded by this gene. Using these primers and a yeast cDNA library (Clontech; No. CL7220-1 derived from S. cerevisiae DBY746) as a template, PCR was carried out.

| | | |
|---|---|---|
| N-terminal primer (Primer 1): | 5′-ATG CCG CCG CTA TTC AAG GGA CT-3′ | (SEQ ID NO: 28) |
| C-terminal primer (Primer 2): | 5′-TTA GGA TTT AAT GCA GGT GAC GG-3′ | (SEQ ID NO: 29) |

The PCR was carried out in the reaction solution as described below under the following conditions: 30 cycles of denaturation at 94° C. for 45 sec, annealing at 55° C. for 1 min and extension at 72° C. for 2 min.

| | |
|---|---|
| 10 × ExTaq buffer (Takara) | 5 μl |
| 2.5 mM dNTP mix | 4 μl |
| 5 U/μl ExTaq (Takara) | 1 μl |
| 10 pmol Primer 1 | |
| 10 pmol Primer 2 | |
| 0.5 ng cDNA | |

To give a 50 μl solution in total

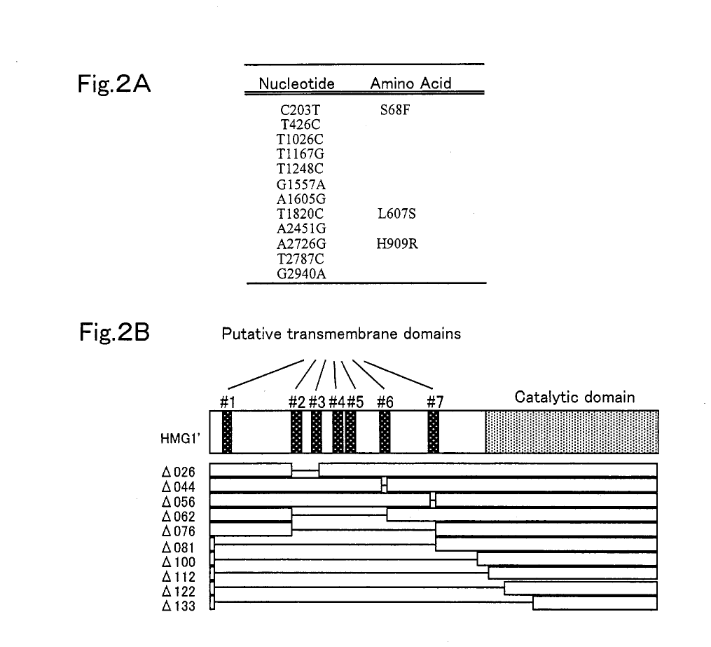

Agarose gel electrophoresis performed after the PCR confirmed a fragment at the expected location (3.2 kbp). This 3.2 kbp DNA fragment was cloned into pT7Blue T vector (Novagen, Madison, Wis.) capable of TA cloning, to thereby obtain pT7HMG1. The nucleotide sequence of the thus cloned HMG-CoA reductase gene was determined. As a result, the nucleotide sequence as shown in SEQ ID NO: 3 and the amino acid sequence as shown in SEQ ID NO: 4 were obtained. The thus determined nucleotide sequence was partially different from the corresponding nucleotide sequence registered in the GenBank (http://www.ncbi.nlm.nih.gov/Genbank/index.html) (FIG. 2A). This gene that comprises PCR errors and encodes the amino acid sequence of a mutant HMG-CoA reductase (SEQ ID NO: 4) is designated HMG1′.

(2) Correction of PCR Errors in HMG1′

An HMG1′ fragment was subcloned from plasmid pT7HMG1 comprising HMG1′ encoding a mutant HMG-CoA reductase. Then, the amino acid substitutions resulted from the PCR errors occurred in the coding region of the wild-type HMG-CoA reductase gene were corrected by site-directed mutagenesis to thereby prepare pALHMG106. The details of this preparation are as described below.

Plasmid pT7HMG1 was used as cloned HMG1′. As a vector for introducing site-directed mutations, pALTER-1 (Promega) was used.

Site-directed mutagenesis was carried out according to the procedures described in “Protocols and Application Guide, 3rd edition, 1996 Promega, ISBN 1-882274-57-1” published by Promega. As oligos for introducing mutations, the following three oligos were synthesized chemically.

| | |
|---|---|
| HMG1 (190-216) | |
| 5′-CCAAATAAAGACTCCAACACTCTATTT-3′ | (SEQ ID NO: 30) |
| HMG1 (1807-1833) | |
| 5′-GAATTAGAAGCATTATTAAGTAGTGGA-3′ | (SEQ ID NO: 31) |
| HMG1 (2713-2739) | |
| 5′-GGATTTAACGCACATGCAGCTAATTTA-3′ | (SEQ ID NO: 32) |

First, pT7HMG1 was digested with Smal, ApaLI and SalI, and a 3.2 kbp HMG1′ fragment was prepared by agarose gel electrophoresis. This fragment was inserted into the SmaI-SalI site of pALTER-1 to prepare pALHMG1. After denaturation of this plasmid with alkali, the above-described oligos for introducing mutations, Amp repair oligo (Promega) as repair oligos, and Tet knockout oligo (Promega) as knockout oligos were annealed thereto. The resultant plasmid was introduced into E. coli ES1301 (Promega). Transformants that retained plasmids into which site-directed mutations had been introduced were selected and cultured with 125 μg/ml ampicillin to prepare plasmid DNA. The nucleotide sequence of the resultant plasmid DNA was examined with primers having the sequences as shown below. As a result, all the sequences corresponding to HMG1 (190-216), HMG1 (1807-1833) and HMG1 (2713-2739) were corrected so that they had the sequences of these oligonucleotides (SEQ ID NO: 5). The amino acid sequence encoded by the corrected nucleotide sequence (SEQ ID NO: 6) was consistent with the amino acid sequence encoded by the wild-type HMG1 (SEQ ID NO: 2); the corrected sequence retained only silent mutations. Since this PCR error-corrected HMG1 encodes a polypeptide having the same amino acid sequence as that of the wild-type enzyme though it has a partially different nucleotide sequence, this gene is also designated HMG1 and used herein without distinction between this and the wild-type gene HMG1.

| | |
|---|---|
| HMG1 (558-532) | |
| 5′-GTCTGCTTGGGTTACATTTTCTGAAAA-3′ | (SEQ ID NO: 33) |
| HMG1 (1573-1599) | |
| 5′-CATACCAGTTATACTGCAGACCAATTG-3′ | (SEQ ID NO: 34) |
| HMG1 (2458-2484) | |
| 5′-GAATACTCATTAAAGCAAATGGTAGAA-3′ | (SEQ ID NO: 35) |

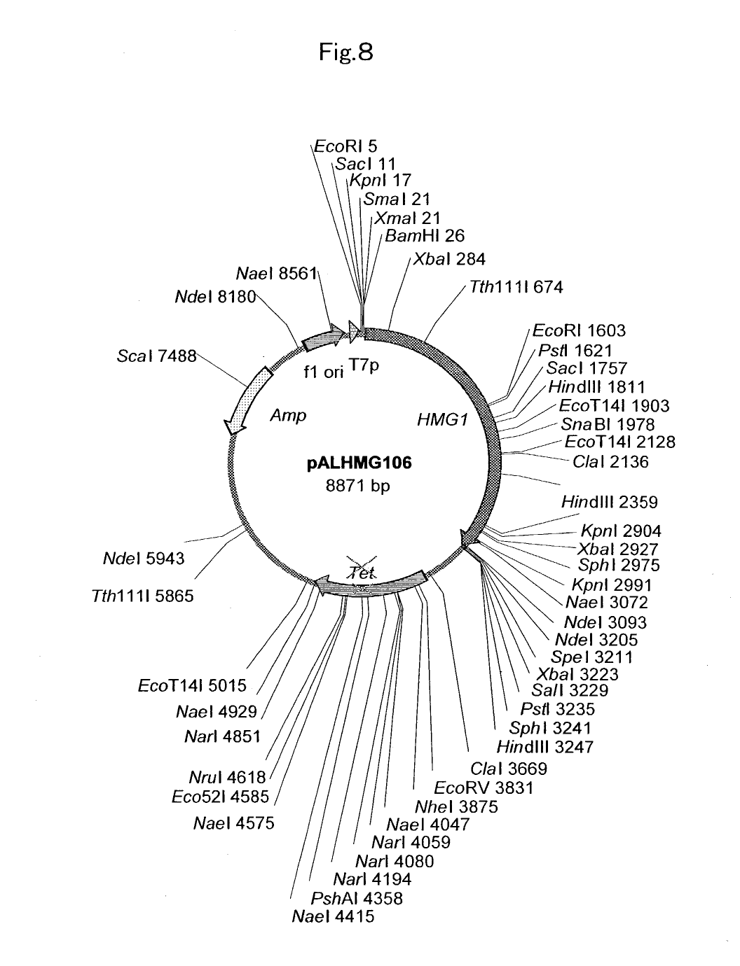

The plasmid carrying the thus corrected HMG1 sequence was designated pALHMG106 (FIG. 8).

(3) Cloning of Geranylgeranyl Diphosphate Synthase Gene BTS1

S. cerevisiae BTS1 gene (also called GGPP synthase gene) was cloned as described below.

Based on information on S. cerevisiae-derived GGPP synthase gene registered in the GenBank (Accession No. U31632) (Y Jiang, et al., J. Biol. Chem. 270 (37), 21793-21799 (1995)), a pair of primers described below matching an N-terminal and a C-terminal region of the enzyme were designed. Using these primers and a yeast cDNA library (CL7220-1) as a template, PCR was carried out.

| | | |
|---|---|---|
| N-teiminal primer: | 5′-ATG GAG GCC AAG ATA GAT GAG CT-3′ | (SEQ ID NO: 36) |
| C-terminal primer: | 5′-TCA CAA TTC GGA TAA GTG GTC TA-3′ | (SEQ ID NO: 37) |

The PCR was performed in a reaction solution having a composition similar to that of the reaction solution described in (1) above under the following conditions: 30 cycles of denaturation at 94° C. for 45 sec, annealing at 55° C. for 1 min and extension at 72° C.for 2 min.

Agarose gel electrophoresis performed after the PCR confirmed a fragment having the proper mobility (corresponding to approx. 1.0 kbp). This BTS1 cloned into pT7Blue T vector capable of TA cloning, followed by sequencing of the entire region of this BTS1 gene. The results revealed that the nucleotide sequence of this gene was completely identical with the nucleotide sequence registered in the GenBank. Thus, it was confirmed that this gene is the S. cerevisiae-derived GGPP synthase gene.

The pT7Blue T vector was digested with BamHI and SalI to cut out the BTS1 gene, which was then introduced into the BamHI-XhoI site of pYES2 (Invitrogen). The recombinant plasmid obtained was designated pYESGGPS.

(4) Cloning of Escherichia coli-derived FPP Synthase Gene ispA

E. coli genomic DNA was prepared from E. coli JM109 (Takara) by the following procedures. JM109 cells were cultured in 1.5 ml of 2×YT medium and harvested by centrifugation. To these cells, 567 μl of TE (pH 8.0), 3 μl of 20 mg/ml proteinase K (Boehringer Mannheim, Mannheim, Germany) and 30 μl of 10% SDS were added. The resultant mixture was left at 37° C. for 1 hr, and then 100 μl of 5M NaCl was added thereto and mixed. Eighty μl of CTAB/NaCl solution (10% CTAB, 0.7 M NaCl) was added thereto, and the resultant mixture was heated at 65° C. for 10 min. This mixture was then treated with 700 μl of chloroform/isoamyl alcohol (24:1) extraction, and a further extraction was carried out with 600 μl of phenol/chloroform/isoamyl alcohol (25:24:1) to the obtained aqueous layer,. which was then centrifuged. The precipitate was washed with 70% ethanol, dried, and then dissolved in 100 μl of TE (pH 8.0) to thereby obtain an E. coli genomic DNA solution. The DNA was measured and quantitatively determined at OD₂₆₀. Then, TE was added to the solution to give a DNA concentration of 1 μg/μl.

Using the thus obtained E. coli genomic DNA as a template and the following synthetic oligo-DNA primers, E. coli-derived FPP synthase gene ispA was cloned by PCR.

(SEQ ID NO: 68)

| | |
|---|---|
| ISPA1: | 5′-TGA GGC AIG CAA TTT CCG CAG CAA CTC G-3′ |

(SEQ ID NO: 69)

| | |
|---|---|
| ISPA2: | 5′-TC AGA ATT CAT CAG GGG CCT ATT AAT AC-3′ |

PCR was carried out in a 100 μl reaction solution containing 133 ExTaq buffer, 0.5 mM dNTP, 100 pmol of ISPA1, 100 pmol of ISPA2, 0.2 μg of E. coli genomic DNA and 5 units of ExTaq under the following conditions: 30 cycles of denaturation at 94° C. for 1 min, annealing at 55° C. for 1 min and extension at 72° C. for 1.5 min. The PCR product was digested with EcoRI and SphI. Then, the resultant 1.0 kbp fragment was purified by agarose gel electrophoresis and inserted into the EcoRI-SphI site of pALTER-Ex2 (Promega), which was then introduced into E. coli JM109 for the cloning of the gene. Restriction enzyme mapping using EcoRI, SphI, NdeI, SmaI, and BamHI revealed that ispA gene (SEQ ID NO: 77) had been introduced correctly into earned three plasmids, i.e., pALispA4, pALispA16 and pALispA 18.

(5) Preparation of Mutant FPP Synthase Genes

Using plasmid pALispA16, the codon encoding the amino acid residue Tyr at position 79 of the polypeptide encoded by E. coli ispA was modified by substitution according to the procedures described in “Protocols and Applications Guide, the 3rd edition, 1996, Promega, ISBN 1-882274-57-1” published by Promega. The following oligos for introducing mutations (also called “mutant oligos”) were prepared by chemical synthesis.

| | | |
|---|---|---|
| ISPA-D: | 5′-ATC ATG AAT TAA TGA GTC AGC GTG GAT GCA TTC AAC GGC GGC AGC-3′ | (SEQ ID NO: 70) |
| ISPA-E: | 5′-ATC ATG AAT TAA TGA TTC AGC GTG GAT GCA TTC AAC GGC GGC AGC-3′ | (SEQ ID NO: 71) |
| ISPA-M: | 5′-ATC ATG AAT TAA TGA CAT AGC GTG GAT GCA TTC AAC GGC GGC AGC-3′ | (SEQ ID NO: 72) |

The above-described mutant oligo ISPA-M was designed so that the nucleotides from position 16 to position 18 (the three nucleotides underlined) encode Met, which nucleotides correspond to the codon for the 79th amino acid residue Tyr in the wild-type gene. Similarly, mutant oligos ISPA-D and ISPA-E were designed so that the corresponding codons encode Asp and Glu, respectively. In these mutant oligos, the nucleotides from position 26 to position 31 (the six nucleotides underlined) were designed so that EcoT221(NsiI) site is newly formed by the substitution mutation. Thus, it is so arranged that these mutant genes can be easily distinguished from the wild-type gene by restriction enzyme mapping. The mutant oligos were treated with T4 polynucleotide kinase (Promega) in advance to phosphorylate their 5′ end and purified by gel filtration with Nick Column (Pharmacia Biotech, Uppsala, Sweden) before use. For the introduction of mutations, Cm repair oligo (Promega) as the repair oligo, and Tet knockout oligo (Promega) as the knockout oligo were also used. Cm repair oligo, Tet knockout oligo and the mutant oligos were annealed to alkali-denatured pALispA16, which was then transformed into E. coli ES1301 mutS (Promega). Plasmid DNA was prepared from E. coli colonies growing in the presence of 20 μg/ml chloramphenicol (Cm), and transformed into E. coli JM109. Plasmid DNA was prepared from E. coli colonies growing on agar plates containing 20 μg/ml Cm. Plasmids containing substitution-mutated ispA genes (designated ispAm genes) that were prepared using pALispA4 as a template and ISPA-D, ISPA-E and ISPA-M as mutant oligos were designated p4D, p4E and p4M, respectively. Those plasmids prepared similarly using pALispA16 as a template were designated p16D, p16E and p16M, respectively. Those plasmids prepared similarly using pALispA18 as a template were designated p18D, p18E and p18M, respectively.

(6) Cloning of IPPA-Isomerase Gene idi

E. coli IPPA-isomerase gene was formerly called as ORF182 (according to NCBI BLAST search; GenBank Accession No. AE000372), but Hahn et al. ((1999) J. Bacteriol., 181: 4499-4504) designated this gene idi. As plasmids in which idi (SEQ ID NO: 85; encoding the amino acid sequence as shown in SEQ ID NO: 86) is cloned, p3-47-11 and p3-47-13 described in Hemmi et al., (1998) J. Biochem., 123: 1088-1096 were used in the invention.

(7) Cloning of Bacillus stearothermophilus FPP Synthase Gene

Plasmid pFE15 described in Japanese Unexamined Patent Publication No. 5-219961 was digested with NotI and SmaI. The resultant 2.9 kbp Bacillus stearothermophilus FPP synthase gene (hereinafter, referred to as “fps”) (SEQ ID NO: 75; encoding the amino acid sequence as shown in SEQ ID NO: 76) fragment containing a transcription unit was purified and inserted into the ScaI site of pACYC177 (Nippon Gene) to obtain plasmid pFE15NS2.9-1.

### EXAMPLE 3

### Insertion of Genes into Expression Vectors

(1) Subcloning into pRS Expression Vectors

HMG1 gene was introduced into individual pRS vectors (FIGS. 6 and 7) prepared in the present invention which are E. coli-S. cerevisiae YEp shuttle vectors containing a constitutive transcription promoter.

pALHMG106 (FIG. 8) containing the PCR error-corrected HMG-CoA reductase gene was digested with SmaI and SalI. The resultant 3.2 kbp HMG1 fragment was purified by agarose gel electrophoresis and inserted into the SmaI-SalI site of pRS434GAP, pRS444GAP, pRS434TEF, pRS444TEF, pRS434PGK and pRS444PGK. Those plasmids into which the gene had been subcloned were examined for their physical maps by restriction enzyme mapping with XhoI, SpeI, NaeI and SphI, and by confirmation of the nucleotide sequences of the border regions of the inserted 3.2 kbp HMG1 fragment. Then, those plasmids created exactly as planned were selected and designated pRS434GAP-HMG1, pRS444GAP-HMG1, pRS434TEF-HMG1, pRS444TEF-HMG1, pRS434PGK-HMG1 and pRS444PGK-HMG1.

(2) Preparation of pRS414PTadh-HMG1 and pRS414TPadh-HMG1

Vectors pRS414PTadh and pRS414TPadh (FIG. 6) containing a constitutive transcription promoter ADH1p were digested with SmaI and SalI, followed by the same operations as described in (1) above. As a result, plasmids pRS414PTadh-HMG1 and pRS414TPadh-HMG1 each containing HMG1 gene inserted thereinto were created.

(3) Preparation of HMG1 ′ Expression Plasmid pYES-HMG1

pT7HMG1 prepared in (1) in Example 2 was digested with BamHI, SalI and ScaI to cut out the HMG1′ gene encoding the mutant HMG-CoA reductase resulted from PCR errors. Then, this gene was inserted into the BamHI-XhoI site of pYES2 (Invitrogen, Carlsbad, Calif.). The resultant recombinant vector was designated pYES-HMG1. As a result of determination of the nucleotide sequence within this vector, it was confirmed that the sequence is identical with the nucleotide sequence as shown in SEQ ID NO: 3. The above plasmid pYES2 is a shuttle vector for expression in yeast that has yeast 2μm DNA ori as a replication origin and GAL1 transcription promoter inducible by galactose (FIG. 4).

(4) Preparation of Deletion Mutant HMG 1′ Expression Plasmid pYHMGxxy

In order to prepare vectors for expressing deletion mutants of HMG-CoA reductase gene having deletion of a nucleotide sequence encoding a region upstream of a domain that is believed to be the catalytic domain of HMG-CoA reductase, a fragment lacking a part of the HMG1′ coding region together with the vector moiety was prepared by PCR using pYES-HMG1 created in (3) above as a template. The resultant fragment was blunt-ended with Klenow enzyme and then circularized again by self-ligation, followed by transformation into E. coli JM109. Then, plasmid DNA was prepared from the transformant. The sequences of the synthetic DNAs used as primers and their combinations are shown in Table 1.

For each of the plasmid DNA obtained, it was confirmed with 373A DNA sequencer (Perkin Elmer, Foster City, Calif.) that there was no shift in the reading frame of amino acids upstream and downstream of HMG1, and that there was no amino acid substitution resulting from PCR errors around the junction site. As a result, the following plasmids were obtained which have no amino acid substitution resulting from PCR errors around the junction site and in which a deletion could be made successively without any shift in the reading frame. Deletion mutants of HMG1 gene are expressed as, e.g., “Δ02y” according to the deletion pattern (where y represents a working number that may be any figure), and pYES2 vectors comprising Δ02y are expressed as, e.g., pYHMG026. (This is applicable to other deletion mutants.)

| | |
|---|---|
| HMG1Δ02y: | SEQ ID NO: 7 |
| HMG1Δ04y: | SEQ ID NO: 8 |
| HMG1Δ05y: | SEQ ID NO: 9 |
| HMG1Δ06y: | SEQ ID NO: 10 |
| HMG1Δ07y: | SEQ ID NO: 11 |
| HMG1Δ08y: | SEQ ID NO: 12 |
| HMG1Δ10y: | SEQ ID NO: 13 |
| HMG1Δ11y: | SEQ ID NO: 14 |
| HMG1Δ12y: | SEQ ID NO: 15 |
| HMG1Δ13y: | SEQ ID NO: 16 |

Vectors: YHMG026, pYHMG027, pYHMG044, pYHMG045, pYHMG062, pYHMG063, PYHMG065, pYHMG076, pYHMG081, pYHMG083, pYHMG085, pYHMG094, pYHMG100, pYHMG106, pYHMG107, pYHMG108, pYHMG109, pYHMG112, pYHMG122, pYHMG123, pYHMG125 and pYHMG133

### EXAMPLE 4

### Preparation of AURGG101

A 1.9 kbp SalI fragment having a primary structure of GAL1 transcription promoter-BTS1-CYC1 transcription terminator (GAL1p-BTS1-CYC1t) was prepared by PCR using pYESGGPS described in (3) in Example 2 as a template and the following primers PYES2 (1-27) and PYES2 (861-835).

| | | |
|---|---|---|
| PYES2 (1-27): | 5′-GGC CGC AAA TTA AAG CCT TCG AGC GTC-3′ | (SEQ ID NO: 73) |
| PYES2 (861-835): | 5′-ACG GAT TAG AAG CCG CCG AGC GGG TGA-3′ | (SEQ ID NO: 74) |

This fragment was inserted into the SalI site of pAUR101 (Takara) to obtain pAURGG115. It was confirmed by DNA sequencing that the BTS1 gene in pAURGG115 had no PCR error.

pAURGG115 was linearized with Eco065I and introduced into A451 strain by the lithium acetate method. Then, colonies growing on YPD agar plates (1% yeast extract, 2% peptone, 2% dextrose, 2% agar) containing lg/ml aureobasidin at 30° C. were selected as transformants. The resultant transformants were cultured again on aureobasidin selection plates to select a single colony.

As a result, two clones AURGG101 and AURGG102 were obtained as recombinants from A451 strain. In the present invention, AURGG101 was used as one of A451-derived host clones.

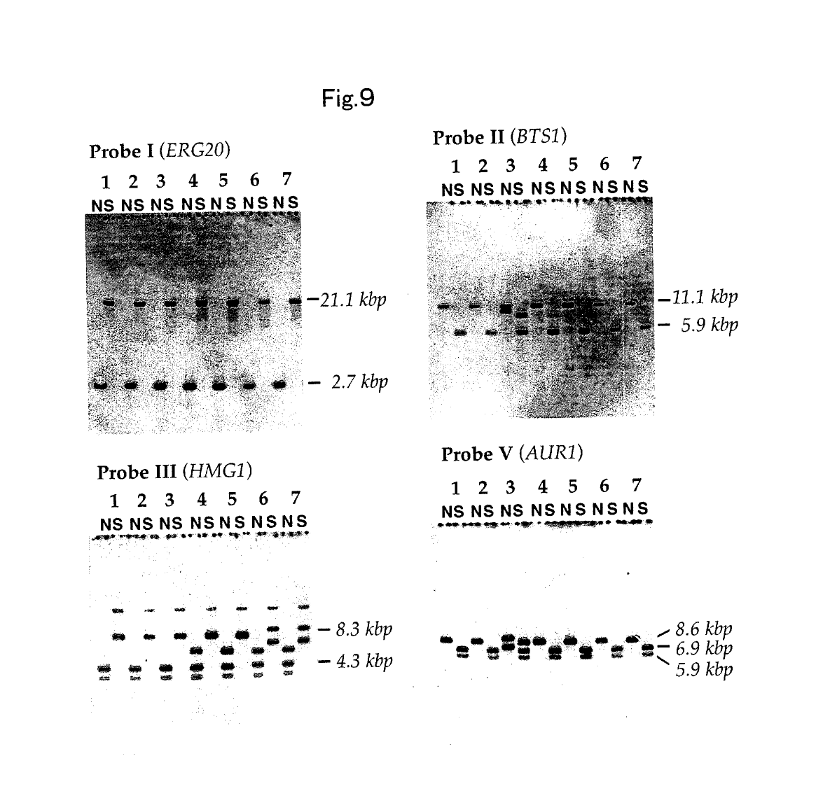

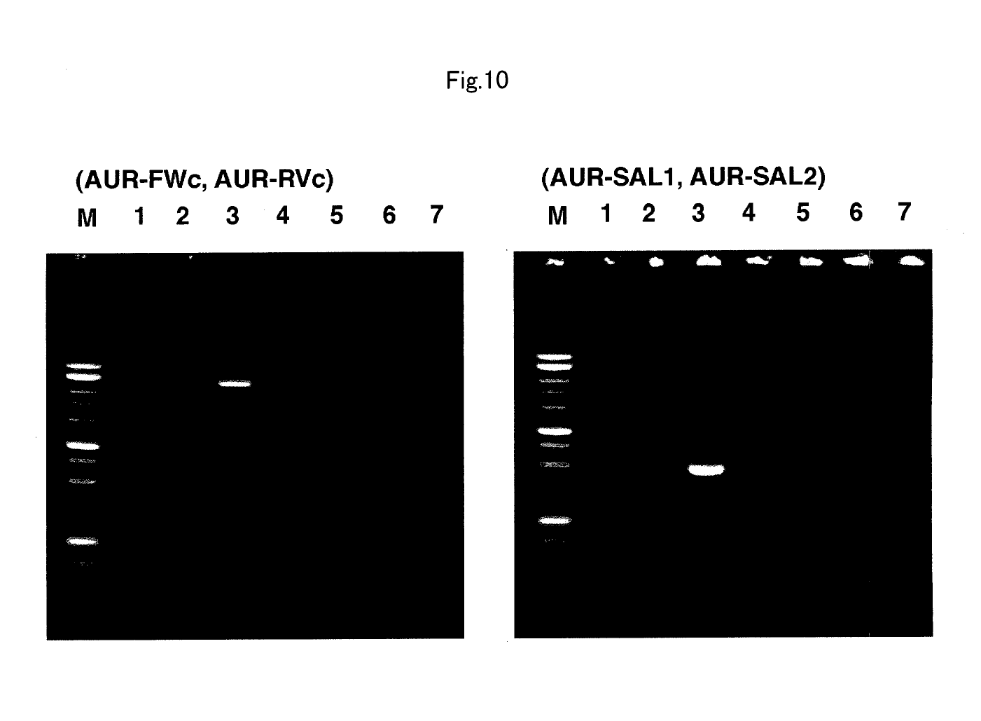

As revealed by Southern blot hybridization (FIG. 9) and PCR mapping (FIG. 10), BTS1 is integrated in the genome in AURGG102 but not integrated therein in AURGG101. In AURGG101, it was found that AUR1 has been merely replaced with AUR1-C (a marker gene). Since AUR1 is not directly involved in the synthesis of prenyl alcohol or prenyl diphosphate, it is possible to use AURGG101 as one example of A451-derived host clones.

Details of the Southern blot hybridization, Northern blot hybridization and PCR mapping are provided in Example 6 described later.

### EXAMPLE 5

### Preparation of EUG Strains

A gene map around squalene synthase gene ERG9 was obtained from a yeast genome database. Based on this map, PCR primer DNAs for amplifying DNA fragments for replacing ERG9 transcription promoter (ERG9p) were designed (FIG. 2). On the other hand, a 1.8 kbp DNA fragment comprising a transformant selection marker gene URA3 and a transcription promoter GAL1p was prepared by PCR amplification using, as a template, pYES2A obtained by digesting pYES2 with NaeI and NheI, blunt-ending with Klenow enzyme and deleting 2μ ori by self-ligation.

The primers used in the PCR are as follows.

| | | |
|---|---|---|
| E-MCSf: | 5′-GCC GTT GAC AGA GGG TCC GAG CTC GGT ACC AAG-3′ | (SEQ ID NO: 49) |
| E-URA3r: | 5′-CAT ACT GAC CCA TTG TCA ATG GGT AAT AAC TGA T-3′ | (SEQ ID NO: 50) |

In the above primers, an Eam1105I recognition site (the underlined portion) is added so that T/A ligation can be conducted by using (i) a 0.7 kbp DNA fragment comprising a downstream portion of the open reading frame YHR189W in the genome of S. cerevisiae and (ii) a 0.9 kbp DNA fragment comprising an upstream portion of ERG9. The YHR189W fragment was prepared by PCR using the following primers YHR189Wf and YHR189Wr, and YPH499 genomic DNA as a template. The ERG9 fragment was prepared by PCR using the following primers ERG9f and ERG9r, and YPH499 genomic DNA as a template. YPH499 genomic DNA was prepared with Dr. GenTLE™.

| | |
|---|---|
| YNIR189Wf: | |
| 5′-TGT CCG GTA AAT GGA GAC-3′ | (SEQ ID NO: 51) |
| YHR189Wr: | |
| 5′-TGT TCT CGC TGC TCG TTT-3′ | (SEQ ID NO: 52) |
| ERG9f: | |
| 5′-ATG GGA AAG CTA TTA CAA T-3′ | (SEQ ID NO: 53) |
| ERG9r: | |
| 5′-CAA GGT TGC AAT GGC CAT-3′ | (SEQ ID NO: 54) |

The 1.8 kbp DNA fragment was digested with Eam1105I and then ligated to the 0.7 kbp DNA fragment. With the resultant fragment as a template, 2nd PCR was carried out using the above-described primers YHR189Wf and E-MCSf. The amplified 2.5 kbp DNA fragment was digested with Eam 1105I and then ligated to the 0.9 kbp fragment. With the resultant fragment as a template, 3rd PCR was carried out using the following primers YHR189W-3f and ERG9-2r. As a result, a 3.4 kbp DNA fragment was amplified. This was used as a DNA fragment for transformation.

| | |
|---|---|
| YHR189W-3f: | |
| 5′-CAA TGT AGG GCT ATA TAT G-3′ | (SEQ ID NO: 55) |
| ERG9-2r: | |
| 5′-AAC TTG GGG AAT GGC ACA-3′ | (SEQ ID NO: 56) |

A vector was introduced into yeast strains using Frozen EZ Yeast Transformation II kit purchased from Zymo Research (Orange, Calif.). The resultant recombinants were cultured on an agar medium called SGR-U medium that had been obtained by adding CSM (-URA) (purchased from BIO 101, Vista, Calif.) and adenine sulfate (final concentration 40 mg/L) to SGR medium (a variation of SD medium in which the glucose component is replaced with galactose and raffinose), at 30° C. Colonies grown on the medium were spread on the same medium again, cultured and then subjected to single colony isolation.

The resultant recombinants were designated EUG (ERG9p::URA3-GAL1p) strain. Of these, clones derived from A451 strain were designated EUG1 through EUG10; clones derived from YPH499 strain were designated EUG11through EUG20; and clones derived from YPH500 strain were designated EUG21through EUG30.

They were cultured on SD medium to select those clones that exhibit growth exhibition as a result of the inhibition of ERG9 expression due to glucose repression. As a result, EUG8 from A451, EUG12 from YPH499 and EUG27 from YPH500 were obtained.

Genomic DNA was prepared from EUG8, EUG12 and EUG27, separately, using Dr. GenTLE™. The results of PCR using the genomic DNA as a template confirmed that the 1.8 kbp PCR fragment containing URA3 and GAL1p is integrated into the genome of each strain upstream of the ERG9 coding region.

### EXAMPLE 6

### Analysis of Genes and Enzyme Activity

In this Example, the expression of genes in various recombinant yeasts prepared in the invention (for the preparation thereof, see Examples 7 and 8 describing the production of prenyl alcohols) was analyzed by determining the enzyme activity of prenyl-diphosphate synthase and by various techniques including Northern blot hybridization, Southern blot hybridization and PCR mapping. Of the prepared recombinants, the host strain and the recombinants listed below were used in this Example. The introduction of individual vectors into the host was carried out according to the lithium acetate method described in Current Protocols in Molecular Biology, John Wiley & Sons, Inc., pp. 13.7.1-13.7.2 or by a method using Frozen EZ Yeast Transformation II kit (Zymo Research, Orange, Calif.) according to the protocol attached to the kit. Clone 1-2 was obtained by introducing pYES-HMG1 into A451; clone 3-2 was obtained by introducing pYHMG044 A451; clone 13-2 was obtained by introducing pYES-HMG1 into AURGG101; and clone 15-2 was obtained by introducing pYHMG044 into AURGG101.

No.1 host strain: A451

No.2 GAL1p-BTS1 (YIp): AURGG101 (A451, aur1::AUR1-C)

No.3 GAL1p-BTS (Y1p): AURGG102 (A451, aur1::BTS1-AUR1-C)

No.4 GAL1p-HMG1 (YEp): 1-2 (pYES-HMG1/A451)

No.5 GAL1p-HMG1Δ (YEp): 3-2 (pYHMG044/A451)

No.6 GAL1p-HMG1 (YEp): 13-2 (pYES-HMG1/AURGG101)

No.7 GAL1p-HMG1Δ (YEp): 15-2 (pYHMG044/AURGG101)

Clones No. 1 to No. 7 were precultured separately at 26° C. One milliliter of the preculture was washed with physiological saline, added to 100 ml of a culture broth and cultured in a 300 ml Erlenmeyer flask at 26° C. with reciprocal shaking at 120 times/min. SD medium or SG medium (in which the glucose component of SD medium is replaced with galactose) was used for the cultivation. Recombinants retaining URA3 marker were cultured in SD-U [CSM (-URA)-added SD medium] or SG-U [CSM (-URA)-added SG medium]. AURGG clones were cultured in the presence of aureobasidin at 1 μg/L.

Cell growth was determined at OD₆₀₀. Cultivation was stopped when the value at OD₆₀₀ reached about 3-4 (23-52 hours). The culture was cooled in ice and then subjected to the preparation of DNA, RNA and crude enzyme solution, as described below.

(1) Southern Blotting

Yeast DNA was prepared using the yeast DNA preparation kit Dr. GenTLE™ according to the protocol attached to the kit.

The DNA thus prepared from yeast was digested with NdeI and StuI, followed by 0.8% agarose gel electrophoresis (3 μg/lane). As molecular weight markers, 0.5 μg each of 1 kb ladder and λ/HindIII (both from Promega, Madison, Wis.) were used. After the electrophoresis, the DNA was denatured with alkali, neutralized and then transferred onto Hybond N nylon membrane (Amersham, Buckinghamshire, England) by capillary blotting with 20×SSC according to conventional methods. The resultant membrane was subjected to UV irradiation with a UV cross-linker (Stratagene) under conditions of optimal cross-linking, to thereby fix the DNA on the membrane.

(2) Northern Blotting

RNA was prepared according to the method described in Current Protocols in Molecular Biology, John Wiley & Sons, Inc., pp. 13.12.2-13.12.3 with partial modification. The modification was that once prepared RNA samples were further treated with DNase I.

After separation of RNA by formaldehyde-denatured agarose gel electrophoresis, the RNA was transferred onto Hybond N nylon membrane by capillary blotting with 20×SSC according to conventional methods. Five micrograms of total RNA was electrophoresed per lane. As a molecular marker, 20 ng of DIG-RNA Marker I was used. The resultant membrane was subjected to UV irradiation with a UV cross-linker (Stratagene) under conditions of optimal cross-linking, to thereby fix the RNA on the membrane.

(3) PCR Mapping

In order to examine how a fragment from pAURGG 115 (a YIp vector prepared in Example 4) is integrated into the genome, PCR was carried out using 0.3-0.6 μg of the yeast DNA prepared above as a template and a combination of synthetic oligonucleotide primers AUR-FWc and AUR-RVc, or AUR-SAL1 and AUR-SAL2. PCR conditions were as follows: 30 cycles of denaturation at 94° C. for 30 sec, annealing at 55° C. for 1 min and extension at 72° C. for 3 min.

| | |
|---|---|
| AUR-FWc: | |
| 5′-TCT CGA AAA AGG GTT TGC CAT-3′ | (SEQ ID NO: 57) |
| AUR-RVc: | |
| 5′-TCA CTA GGT GTA AAG AGG GCT-3′ | (SEQ ID NO: 58) |
| AUR-SAL1: | |
| 5′-TGT TGA AGC TTG CAT GCC TGC-3′ | (SEQ ID NO: 59) |
| AUR-SAL2: | |
| 5′-TTG TAA AAC GAC GGC CAG TGA-3′ | (SEQ ID NO: 60) |

(4) Preparation of DIG-Labeled Probe DNAs

As hybridization probes, Probes I, II, III and V were prepared (Table 5).

TABLE 5
Hybridization Probes

| Probe No. | Gene | Template | Primer 1 | Primer 2 |
|---|---|---|---|---|
| I | ERG20 | pT7ERG20 | SCFPS1 | SCFPS2 |
| II | BTS1 | pYES2-GGPS6 | BTS1 | BTS1 |
| | | | (1-21) | (1008-982) |
| III | HMG1 | pYHMG1 | HMG1 | HMG1 |
| | | | (1267-1293) | (2766-2740) |
| V | AUR1 | pAUR123 | AUR-RV | AUR-FW |

Probe I:

Using the following synthetic oligonucleotides SCEPS1 and SCEPS2 as primers, a PCR fragment was obtained from an S. cerevisiae cDNA library (Clontech, Palo Alto, Calif.) and cloned into pT7blue T vector. Thus, pT7ERG20 was prepared.

(SEQ ID NO: 61)

| | |
|---|---|
| SCEPS1: | 5′-ATG GCT TCA GAA AAA GAA ATT AG-3′ |

(SEQ ID NO: 62)

| | |
|---|---|
| SCFPS2: | 5′-CTA TTT GCT TCT CTT GTA AAC TT-3′ |

Using pT7ERG20 as a template and SCEPS1 and SCEPS2 as primers, a DIG (digoxigenin)-labeld probe DNA was synthesized with PCR DIG Probe Synthesis Kit (Roche Diagnostics, Mannheim Germany). Experimental conditions were in accordance with the manufacturer's protocol attached to the kit.

PCR conditions were as follows: 30 cycles of denaturation at 94° C. for 30 see, annealing at 58° C. for 1 min and extension at 72° C. for 3 min. The resultant DIG-labeled probe DNA was subjected to agarose gel electrophoresis to examine the state of synthesis.

Probe II:

A DIG-labeled probe DNA was synthesized in the same manner as used for Probe I, using the following synthetic oligonucleotides as primers and pYESGGPS (see (3) in Example 2) as a template.

BTS1 (1-21):
5′-ATG GAG GCC AAG ATA GAT GAG-3′ (SEQ ID NO: 63)
BTS1 (1008-988):
5′-TCA CAA TTC GGA TAA GTG GTC-3′ (SEQ ID NO: 64)

Probe III:

A DIG-labeled probe DNA was synthesized in the same manner as used for Probe I, using the following synthetic oligonucleotides as primers and pYES-HMG1 (see (3) in Example 3) as a template.

HMG1 (1267-1293): 5′-AAC TTT GGT GCA AAT TGG GTC AAT GAT-3′(SEQ ID NO: 42)
HMG1 (2766-2740): 5′-TCC TAA TGC CAA GAA AAC AGC TGT CAC-3′(SEQ ID NO: 65)

Probe V:

A DIG-labeled probe DNA was synthesized in the same manner as used for Probe I, using the following synthetic oligonucleotides as primers and pAUR123 (Takara) as a template.

AUR-FW:
5′-ATG GCA AAC CCT TTT TCG AGA-3′ (SEQ ID NO: 66)
AUR-RY:
5′-AGC CCT CTT TAC ACC TAG TGA-3′ (SEQ ID NO: 67)

(5) Hybridization and Detection of Probes

Southern blot hybridization was carried out at a probe concentration of 20 ng/ml at 42° C. for 24 hr using DIG Easy Hyb (Roche). Northern blot hybridization was carried out at a probe concentration of 100 ng/ml at 50° C. for 24 hr using DIG Easy Hyb. Prior to each hybridization, prehybridization was carried out for 24 hr in DIG Easy Hyb solution at the same temperature used for the hybridization. After the hybridization, the membrane was washed 3 times with 2× SSC, 0.1% SDS at 65° C. for 10 min each, and then 2 times with 0.2× SSC, 0.1% SDS at 65° C. for 15-20 min each. Thereafter, the DIG-labeled probe in the membrane was allowed to generate chemiluminescence by using DIG Luminescent Detection Kit (Roche), followed by exposure of the blot to X-ray film for visualization.

(6) Determination of Enzyme Activity

Cells were harvested from each culture broth by centrifugation and disrupted at 4° C. with glass beads in the same manner as in the preparation of RNA. Then, cells were suspended in sterilized water. The suspension was centrifuged at 12,000 r.p.m. for 10 min with a refrigerated microcentrifuge, and the resultant supernatant was recovered as a crude enzyme fraction. The protein concentration in the crude enzyme fraction was determined by Bio-Rad Protein Assay (Bio-Rad, Hercules, Calif.) using BSA as a standard protein. Ten μg of the crude enzyme fraction was reacted in 200 μl of the following reaction cocktail at 37° C. for 40 min.

```
0.125 mM [¹⁴C] IPP (185 GBq/mol)
0.125 mM geranyl diphosphate (Sigma Chemical, St. Louis, MO)
  100 mM Tris HCl (pH 7.0)
  10 mM NaF, 5 mM MgCl₂
    5 mM 2-mercaptoethanol
  0.05% Triton X-100
0.005% BSA
```

After the reaction, extended prenyl diphosphate was extracted with water-saturated butanol. An aliquot of the prenyl diphosphate was subjected to determination of radioactivity with a liquid scintillation counter. The remaining sample was dephosphorylated with potato acid phosphatase, spotted onto thin layer chromatography plate [plate: LKC 18 (Whatman, Clifton, N.J.], and then the plate was developed [developer solvent: H₂O/acetone=1:19]. The autoradiogram was visualized with Bio Image Analyzer BAS2000 (Fuji Film) and the relative radioactivities were determined, according to the method of Koyama et al. (Koyama T., Fujii, H. and Ogura, K., 1985, Meth. Enzymol. 110:153-155).

(7) Results and Observations

(7-1) Southern Blot Hybridization and PCR Mapping

The results of southern blot hybridization are shown in FIG. 9. The results of PCR mapping in the vicinity of AUR1 are shown in FIG. 10. In FIGS. 9 and 10, lanes 1 to 7 correspond to the numbers of clones (No. 1 to No. 7) used in (6). “N” represents DNA digested with NdeI; and “S” represents DNA digested with StuI. DNAs used in individual lanes were prepared from the following strains.

Lane 1: A451; Lane 2: AURGG101; Lane 3: AURGG102; Lane 4: pYES-HMG1/A451; Lane 5: pYHMG044/A451; Lane 6: pYES-HMG1/AURGG101; Lane 7: pYHMG044/AURGG101

It was found that ERG20 (FPP synthase gene) is contained in the same manner in all of the clones tested and that there is no change in the vicinity of ERG20 in the genome of each clone (FIG. 9).

When BTS1 (GGPP synthase gene) and AUR1 were used as probes, it was found that BTS1 is integrated into the region of AUR1 in AURGG102, but the bands appearing in AURGG101 are the same as those appearing in the host strain A451. In AuRGG101, only AUR1 gene is replaced with pAUR101-derived AUR1-C gene; it was found that the GAL1-BTS1 fragment is not integrated into the genome of this clone. Duplication of AUR1 locus resulting from genomic integration was detected by PCR. As expected, a band was not detected in AURGG101 but detected only in AURGG102 (FIG. 10).

In FIG. 9, when HMG1 was used as a probe, a plasmid-derived band appeared in NdeI-digested DNAs (lanes 4-7). In StuI-digested DNAs, it is expected that a 8.2 kbp band derived from the plasmid (overlapping a 8.3 kbp band derived from the genome) should appear as in clone 1-2 (No. 4). However, a band shift was observed in clone 13-2 (No. 6) and clone 15-2 (No. 7) as a result of recombination between the vicinity of HMG1 in the genome and the plasmid introduced.

From the results of Southern blot hybridization and PCR mapping, the genotypes of the clones used this time can be summarized as shown in Table 6 below. In this Table, “AUR” means a medium to which aureobasidin has been added. “Medium 1” means a medium for preculture, and “Medium 2” means a medium for subsequent culture.

TABLE 6

| | | Inte- | | | |
|---|---|---|---|---|---|
| Clone | | grated | Gene in | | |
| No. | Designation | Gene | Plasmid | Medium 1 | Medium 2 |
| 1 | A451 | — | — | SD | SG |
| 2 | AURGG101 | — | — | SD-AUR | SG-AUR |
| 3 | AURGG102 | BTS1 | — | SD-AUR | SG-AUR |
| 4 | 1-2 | — | HMG1 | SD-U | SG-U |
| 5 | 3-2 | — | HMG1Δ044 | SD-U | SG-U |
| 6 | 13-2 | — | HMG1 | SD-U-AUR | SG-U-AUR |
| 7 | 15-2 | — | HMG1Δ044 | SD-U-AUR | SG-U-AUR |

(7-2) Northern Blot Hybridization

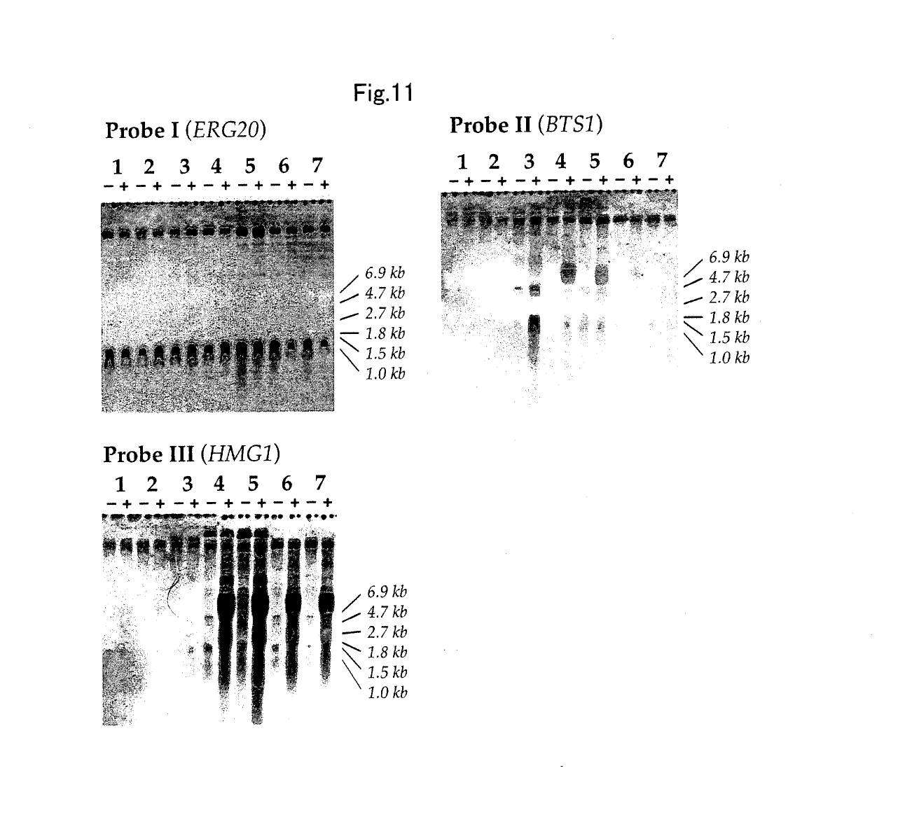

The results of Northern blot hybridization are shown in FIG. 11. Probes I, II and III as shown in Table 5 were used as probe.

In FIG. 11, the clones used in lanes 1 to 7 are the same as used in FIG. 9. Mark “−” indicates transcripts in SD medium, and mark “+” indicates transcripts in SG medium.

ERG20 transcript showed a tendency to decrease in clone 13-2 (No. 6) and clone 15-2 (No. 7) when GAL1p transcriptional induction was applied by SG medium.

When the transcription of genes under the control of GAL1 transcription promoter was induced by SG medium, the induction of BTS1 transcript increased only in a clone in which GAL1p-BTS1 fragment has been integrated into the genome, i.e., AURGG102 (No. 3).

However, when compared with HMG1 transcript, it is seen that the degree of transcription induction of BTS1 is lower. When transcription was induced by SG medium, HMG1 transcript increased remarkably in clones No.4 to No. 7 in which GAL1p-HMG1 fragment has been transferred by a plasmid.

(7-3) Prenyl-Diphosphate Synthase Activity

The activity of prenyl-diphosphate synthase in the crude enzyme fraction was determined using geranyl diphosphate (GPP) and [¹⁴C]-labeled IPP as allylic diphosphate substrates.

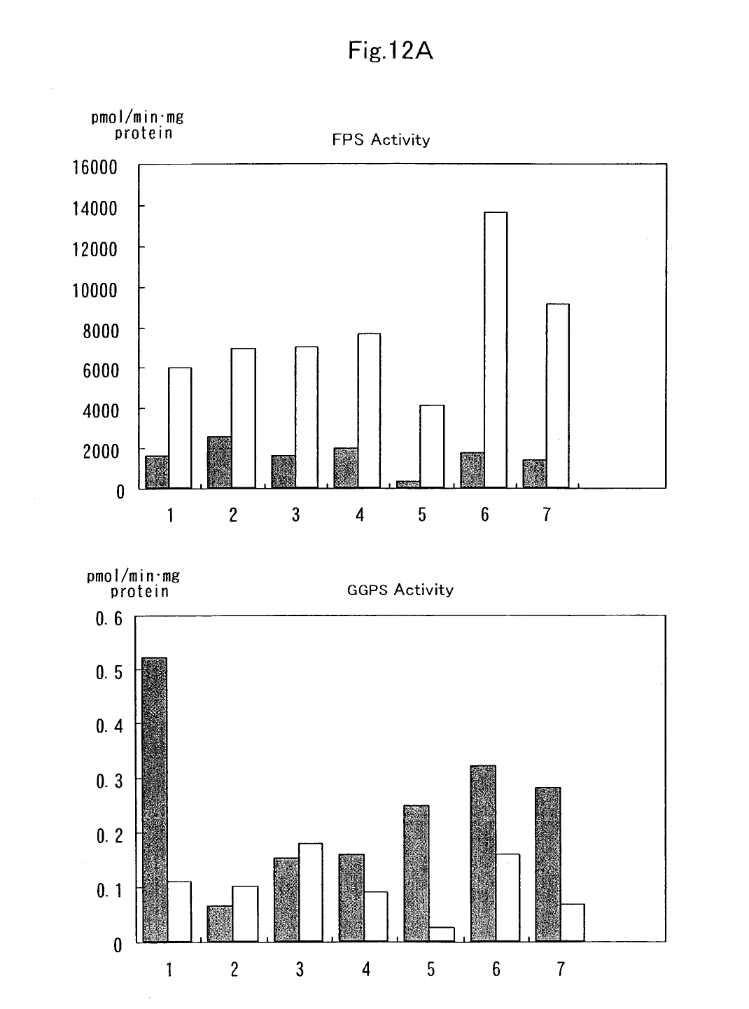

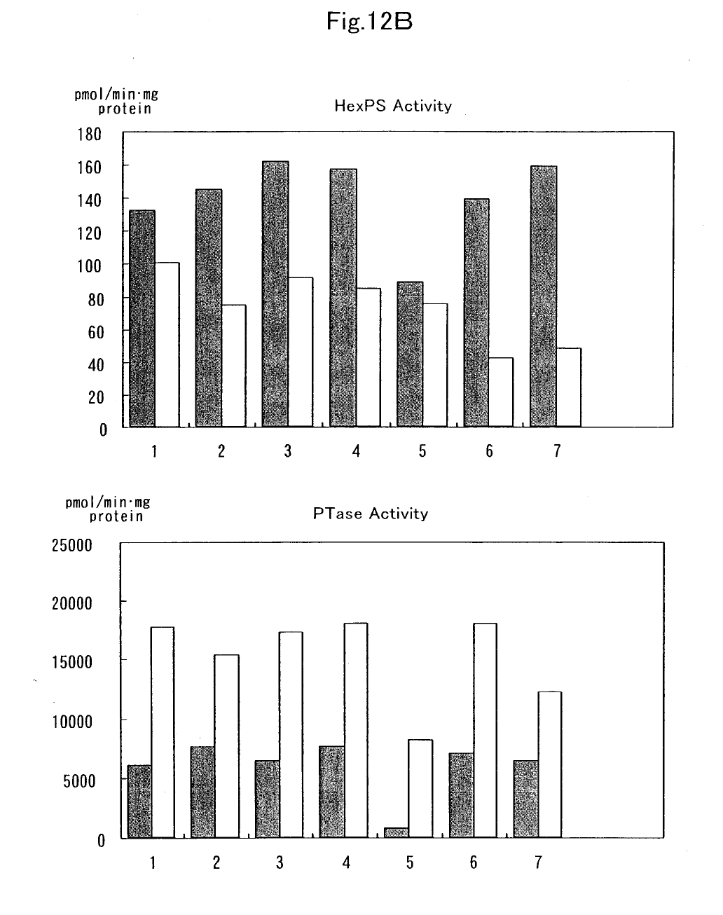

The prenyl diphosphates synthesized with GPP and [¹⁴C] IPP as substrates were dephosphorylated and analized by TLC. Then, the ratioactivity of each spot on the plate was examined. As a result, FPP synthase activity was high, and next to that, HexPP (hexaprenyl diphosphate) synthase activity was detected that was by far higher than GGPP synthase activity. Then, relative amounts of reaction products were calculated from autoradiogram, followed by calculation of specific activity per gross protein. The results are shown in FIG. 12. In FIG. 12A, the upper panel shows FPP synthase (FPS) activity, and the lower panel shows GGPP synthase (GGPS) activity. In FIG. 12B, the upper panel shows HexPP synthase (HexPS) activity, and the lower panel shows PTase (total prenyl-diphosphate synthase) activity. Gray columns show the results in SD medium, and white columns show the results in SG medium. A large part of the total prenyl-diphosphate synthase activity is FPP synthase activity. An increase in this activity caused by SG medium was observed. In particular, total prenyl-diphosphate synthase activity remarkably increased in clone 13-2 (No. 6) and clone 15-2 (No. 7) that produce FOH in a large quantity (see Example 9). As a whole, when GPP is used as an allylic substrate, GGPP synthase activity is about 1/20000 of FPP synthase activity and about 1/300 of HexPP synthase activity. HexPP synthase activity decreased in SG medium.

### EXAMPLE 7

### Cultivation of Recombinants and Production of Prenyl Alcohols

(1) Production of Prenyl Alcohols When HMG1 Gene with a Constitutive Promoter was Introduced into A451 (Such a Recombinant is Expressed as “Constitutive Promoter; HMG1; A451”; This Way of Expression Applies to the Remaining Part of the Specification).

For industrial application of FOH high yielding recombinants, the use of a constitutive promoter is advantageous since it allows the use of cheap, conventional media. Then, HMG1 gene was expressed under the control of a constitutive promoter using as a host S. Cerevisiae A451 (ATCC200589) that was recognized in preliminary experiments to have potentiality to produce FOH.

HMG1 gene (PCR error-corrected gene) was introduced into vector pRS434GAP or pRS444GAP each containing a constitutive promoter GAPp (=TDH3p) to thereby prepare pRS434GAP-HMG1 and pRS444GAP-HMG1, respectively. These plasmids were introduced into A451 to obtain recombinants, which were designated pRS434GAP-HMG1/A451 and pRS444GAP-HMG1 /A451.

Ten colonies were selected randomly from each of the yeast recombinants into which HMG-CoA reductase gene had been introduced. These colonies were inoculated into SD-W medium [obtained by adding CSM (-TRP) to SD] that is an SD selection medium for a marker gene TRP1, and precultured therein. Then, 250 μl of the preculture (when a yeast recombinant with a constitutive promoter was precultured, this amount was added not only in this experiment but in other experiments described later) was added to 2.5 ml of YM medium and cultured at 26° C. for 4 days with rotary shaking at 130 r.p.m.

After completion of the cultivation, 2.5 ml of methanol was added to the culture broth and mixed. Then, about 5 ml of pentane was added thereto and agitated vigorously. The resultant mixture was left stationary. The pentane layer was transferred into a new glass tube, followed by evaporating the pentane in a draft to thereby concentrate solute components. Then, the resultant solution was subjected to gas chromatography/mass spectrography (GC/MS) to identify prenyl alcohols and quantitatively determine them with undecanol as an internal standard. At that time, in order to examine the degree of cell growth, 50 μl of the culture broth was diluted 30-fold with water, followed by determination of absorbance at 600 nm.

GC/MS was carried out with HP6890/5973 GC/MS system (Hewlett-Packard, Wilmington, Del.). The column used was HP-5MS (0.25 mm×30 m; film thickness 0.25 μm). Analytical conditions were as described below. The same conditions were used for all the GC/MS analyses in this specification.

| Inlet temperature: | 250° C. |
|---|---|
| Detector temperature: | 260° C. |
| [MS zone temperatures] | |
| MS Quad: | 150° C. |
| MS Source: | 230° C. |
| Mass scan range: | 35-200 |
| [Injection parameters] | |
| Automated injection mode | |
| Sample volume: | 2 μl |
| Methanol washing: | 3 times; |
| hexane washing: | twice |
| Split ratio: | 1/20 |
| Carrier gas: | helium 1.0 ml/min |
| Solvent retardation: | 2 min |
| [Oven heating conditions] | |
| 115° C. for 90 sec | |
| Heating up to 250° C. at 70° C./min and retaining for 2 min | |
| Heating up to 300° C. at 70° C./min and retaining for 7 min | |
| After Time 0 | |
| Internal standard: | 0.01 μl of 1-undecanol in ethanol |
| Reliable standards: | (E)-Nerolidol (Eisai) |
| | (all-E)-Farnesol (Sigma) |
| | (all-E)-Geranylgeraniol (Eisai) |
| | Squalene (Tokyo Kasei Kogyo) |

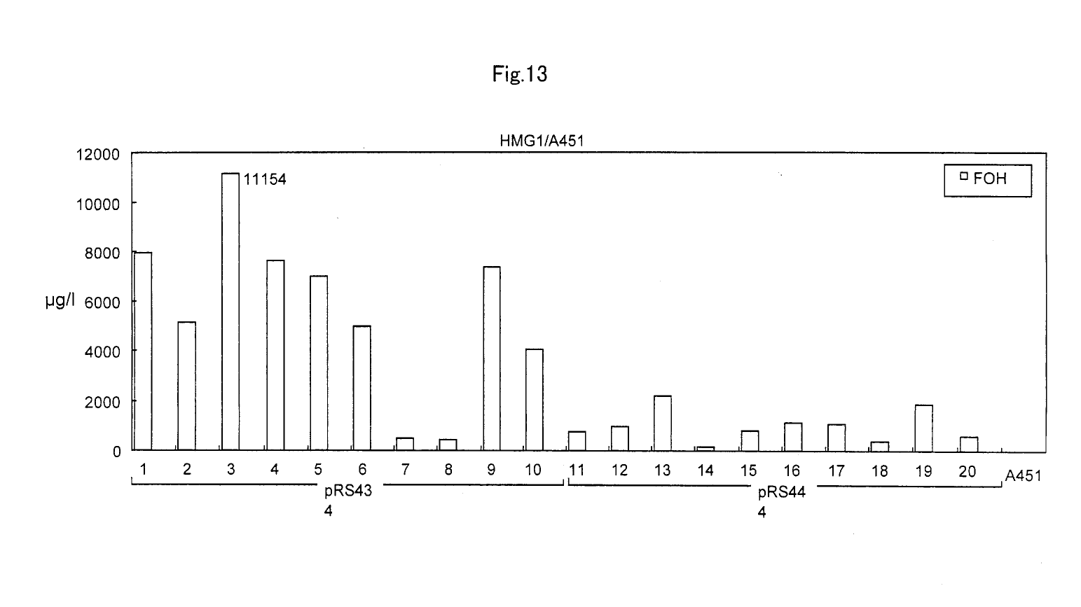

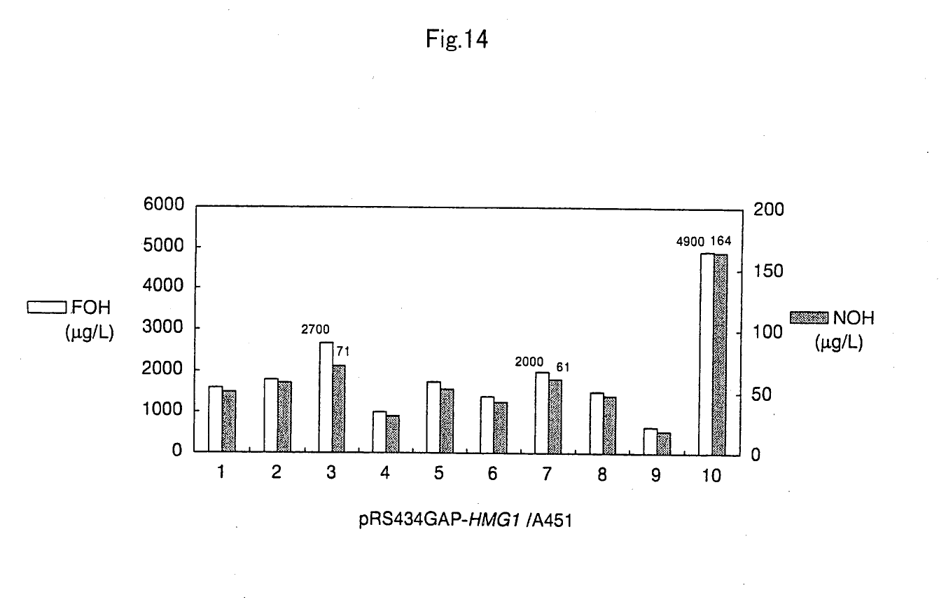

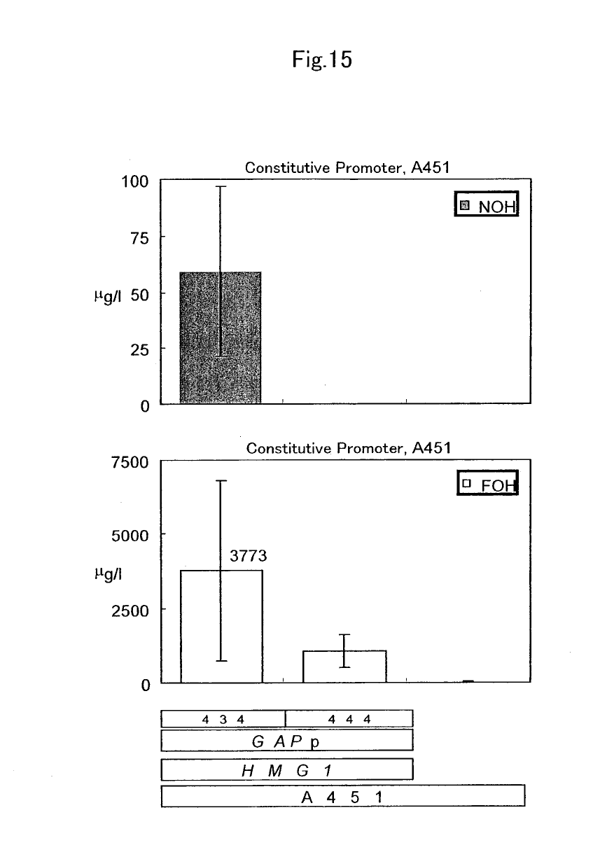

The results of determination of prenyl alcohol yields are shown in FIGS. 13-15. FIG. 14 shows a result selecting 10 colonies from clone No. 3 of pRS434 shown in FIG. 13. FIG. 15 shows a summary of data shown in FIG. 13. An FOH yield of 4.9 mg/L was recognized in colony No. 10 (pRS434) in FIG. 14. In FIG. 15, “434” and “444” represent the results when pRS434GAP and pRS444GAP vectors were used, respectively. The column at the utmost right represents the results when the host (A451) before gene transfer was cultured.

These results revealed that, when A451 was used as a host, the productivity of both NOH and FOH increased in pRS434GAP-HMG/A451. FOH could be produced at 3.8 mg/L on the average, with 11.2 mg/L at the highest, by merely activating the transcription of HMG1 gene (FIG. 13). In pRS444GAP-HMG1/A451, the yield of NOH was 0.16 mg/L at the highest; this clone was found to be effective mainly in the production of FOH.

It is believed that A451 is different from conventionally used recombinant DNA host strains (such as YPH499) in the balance between squalene synthase activity and mevalonate pathway activity, and that farnesyl diphosphate (FPP), an intermediate metabolite, is accumulated when multiple copies of HMG1 gene are present or the transcription of this gene is activated; as a result, FOH (a dephosphorylated product of FPP) is produced. Alternatively, it is believed that the ability to produce FOH was rendered to A451 as a result of mutation of CAN1 or ARO7 seen in the genotype of A451. This means that any strain having a balance similar to that of A451 between squalene synthase activity and mevalonate pathway activity, or any strain having mutation in CAN1 and/or ARO7 can be expected to produce FOH upon introduction of HMG1. With respect to FOH production, a tendency was observed that the use of pRS434GAP vector exhibits better productivity than pRS444GAP vector.

(2) Constitutive Promoter; HMG1; YPH499

The plasmids listed below that had been obtained by inserting HMG1 gene (PCR error-corrected gene) into vector pRS414PTadh, pRS414TPadh, pRS434GAP, pRS444GAP, pRS434PGK, pRS444PGK, pRS434TEF or pRS444TEF comprising a constitutive promoter ADH1p, GAPp (=TDH3p), PGK1p or TEF2p, were introduced into YPH499.

pRS414PTadh-HMG1

pRS414TPadh-HMG1

pRS434GAP-HMG1

pRS444GAP-HMG1

pRS434PGK-HMG1

pRS444PGK-HMG1

pRS434TEF-HMG1

pRS444TEF-HMG1

The resultant recombinants were cultured in YM medium supplemented with adenine sulfate at 40 μg/ml (the same medium was also used for other recombinants when YPH499 was used as a host). Culture conditions were the same as in (1) above. After completion of the cultivation, the pentane extract fraction from the culture broth was subjected to GC/MS analyses. The yields of prenyl alcohols (NOH and FOH) were determined.

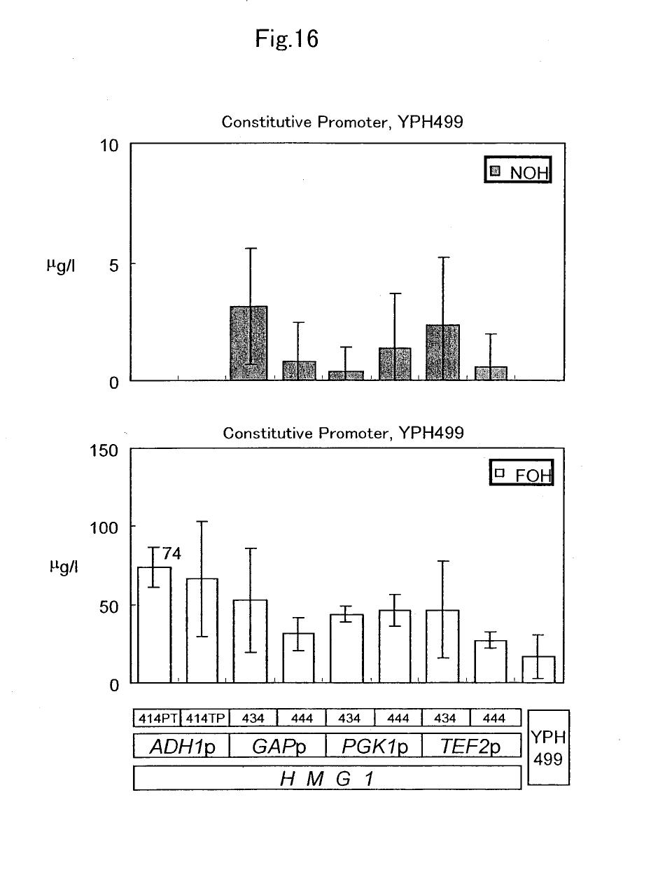

The results are shown in FIG. 16. In FIG. 16, “414PT”, “414TP”, “434” and “444” represent the results when pRS414PTadh, pRS414TPadh, pRS434xxx and pRS444xxx (where xxx indicates the alphabetical part of the name of the gene used in the promoter) vectors were used, respectively. The right utmost column represents the results when the host (YPH499) before gene transfer was cultured. As shown in FIG. 16, the yield of FOH is improved in every recombinant, and an increase in NOH productivity is observed in pRS434GAP-HMG1-, pRS444GAP-HMG1-, pRS434TEF-HMG1-, pRS444TEF-HMG1-, pRS434PGK-HMG1- or pRS444PGK-HMG1-introduced YPH499 clone.

(3) Constitutive Promoter; HMG1; EUG

A451-, YPH499- or YPH500-derived EUG clones that exhibit Glc growth inhibition and have integrated the DNA of interest into the genome completely were selected (i.e., EUG8, EUG12 and EUG27). Then, plasmid pRS434GAP-HMG1 or pRS444GAP-HMG1 obtained by inserting HMG1 gene (PCR error-corrected gene) into vector pRS434GAP or pRS444GAP comprising a constitutive promoter GAPp (=TDH3p) was introduced into EUG8 (NOH yield: 0.021 mg/L; FOH yield: 0.20 mg/L), EUG12 (NOH yield: 0.13 mg/L; FOH yield: 5.9 mg/L) and EUG27 (NOH yield: 0.038 mg/L; FOH yield: 3.2 mg/L). The yields of prenyl alcohols in the resultant recombinants were determined.

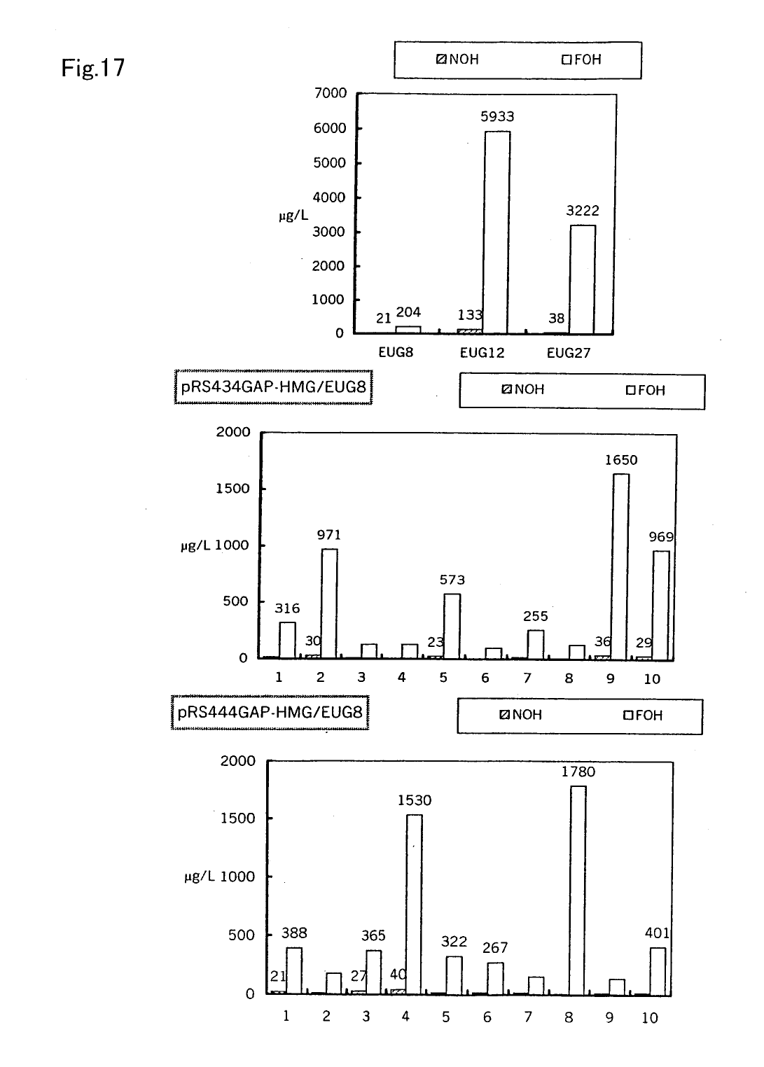

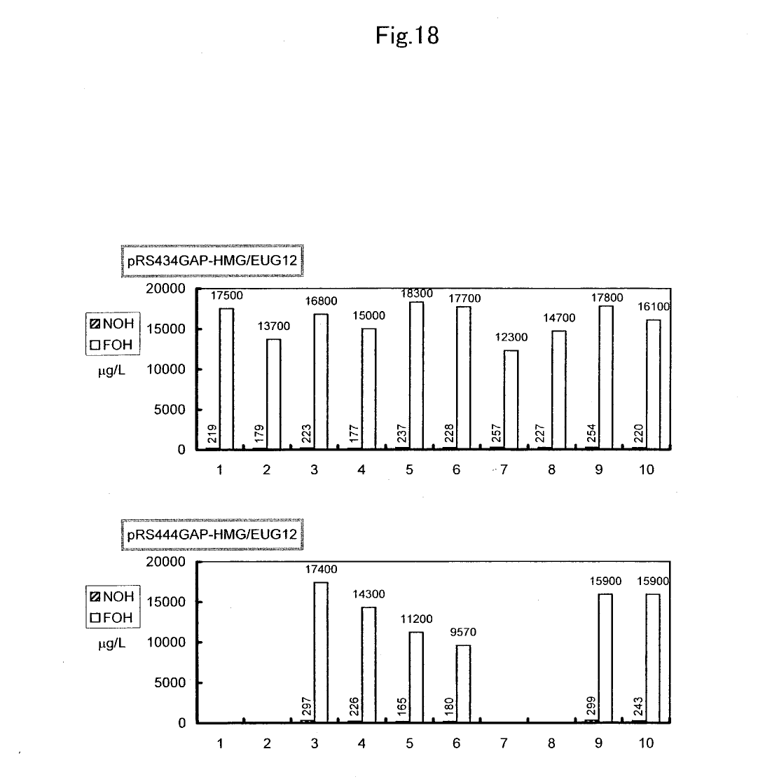

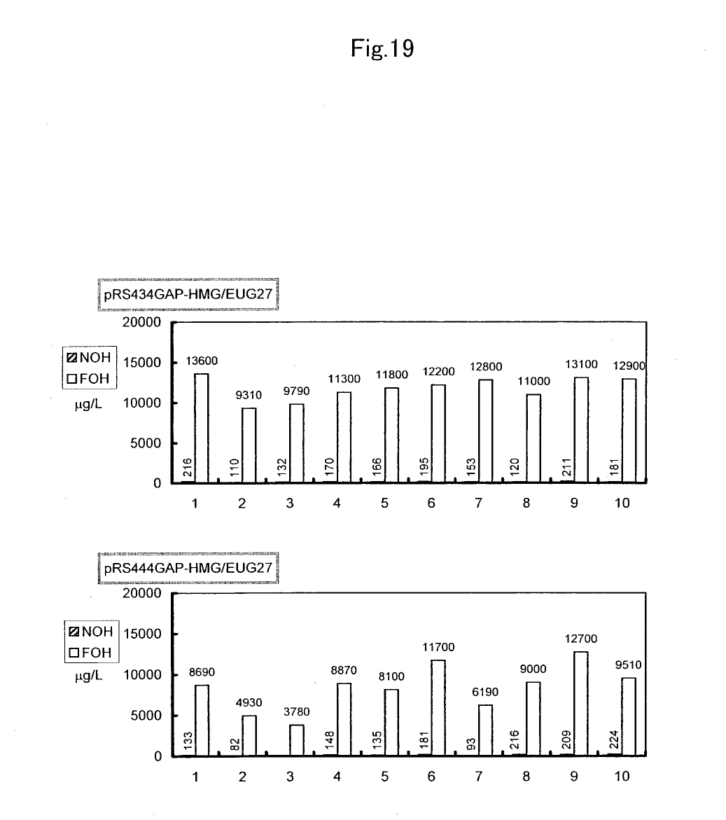

The results are shown in FIG. 17 (EUG8), FIG. 18 (EUG12) and FIG. 19 (EUG27).

EUG clones produce FOH when cultured in YM medium containing glucose (Glc) as the carbon source. The introduction of HMG1 gene improved the productivity of FOH. A451-derived EUG8 is different from YPH499-derived EUG12 and YPH500-derived EUG27 in production profile. It is believed that clones derived from YPH strains are more suitable for production.

These results revealed that it is possible to improve the productivity of prenyl alcohols in A451-derived clones, YPH499-derived clones and YPH500-derived clones by introducing HMG1 thereinto.

(4) Inducible Promoter; HMG1; A451 or AURGG101

Plasmid pYES2-HMG obtained by inserting HMG1′ (a PCR error mutant of HMG1) into vector pYES2 comprising an inducible promoter GAL1p was introduced into A451 and AURGG101 (A451, aur1::AUR1-C) prepared in Example 4.

Each of the resultant recombinants was precultured. Then, 25 μl of the preculture (when a yeast recombinant with an inducible promoter was precultured, this amount was added not only in this experiment but in other experiments described later) was added to 2.5 ml of SG medium and cultured at 26° C. for 4 days with rotary shaking at 130 r.p.m. Prior to the addition to SG medium, cells were washed with physiological saline so that no glucose component was brought into SG medium. After completion of the cultivation in SG medium, the yields of prenyl alcohols (NOH and FOH) were determined.

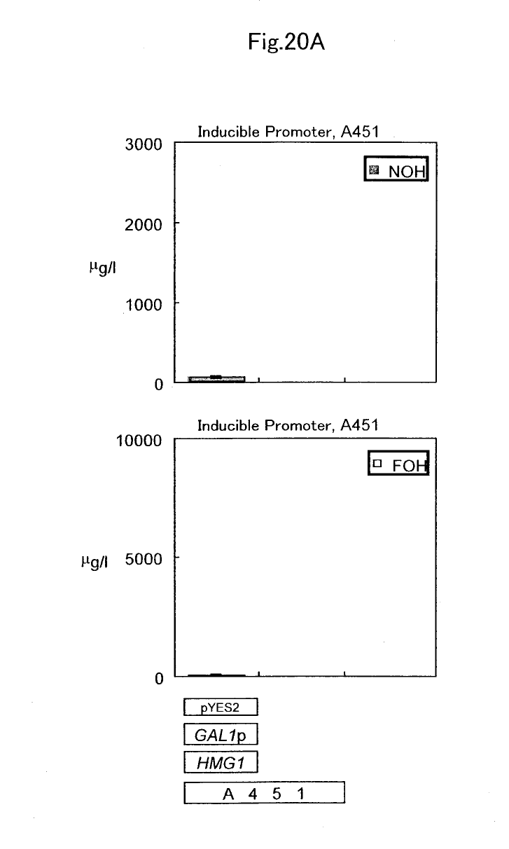

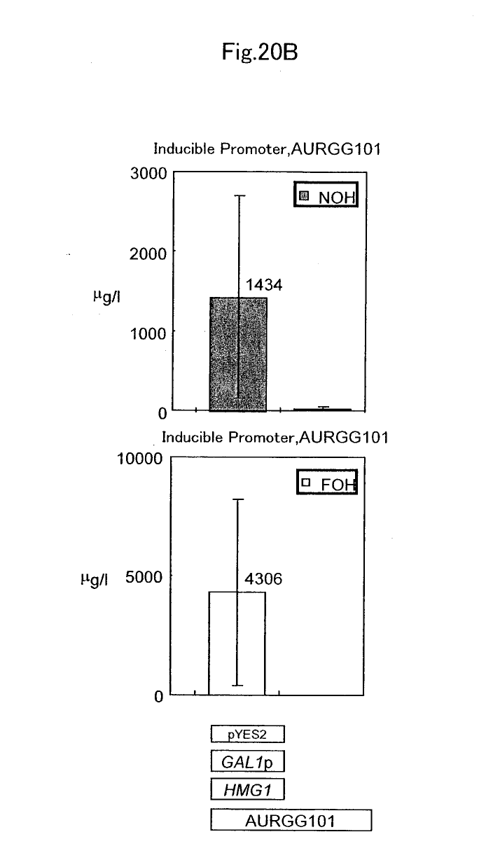

As a result, pYES-HMG1/AURGG101 clones produced NOH at 1.43 mg/L on the average and FOH at 4.31 mg/L on the average. Thus, prenyl alcohol high yielding clones were obtained even in those recombinants in which pYES-HMG1 comprising HMG1′ (a mutant MMG1) has been transferred (FIG. 20). FIG. 20A shows the results when A451 was used. FIG. 20B shows the results when AURGG101 was used. pYES is a vector that was used for the gene transfer.

When AURGG11 derived from A451 was used as a host and GAL1p as a promoter, clones were obtained that highly produced FOH in particular.

(5) Inducible Promoter; HMG1; W303-1A or W303-1B

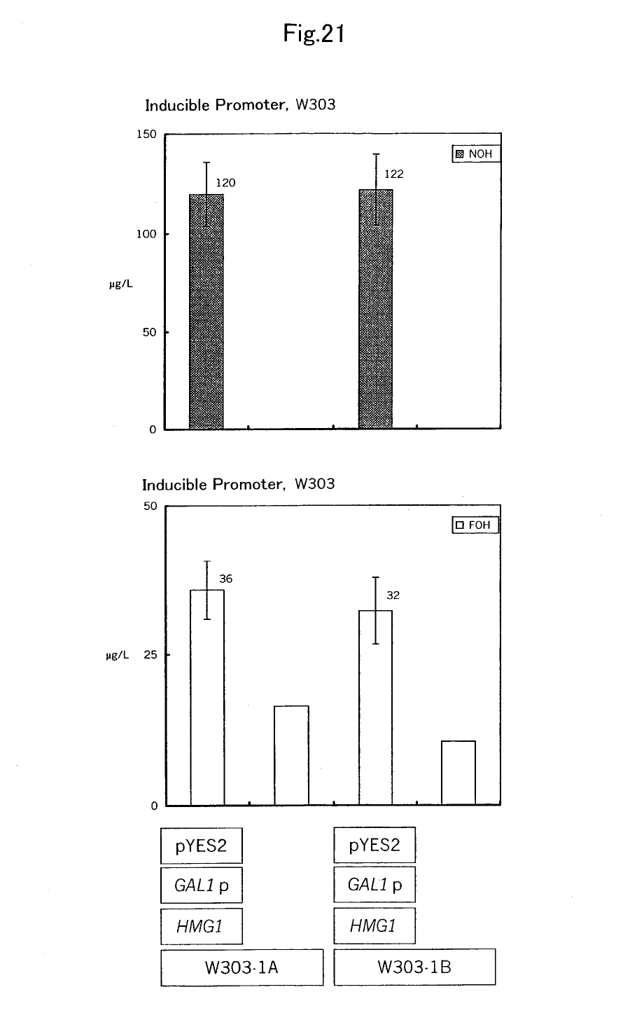

Plasmid pYES2-HMG obtained by inserting HMG1 into vector pYES2 comprising an inducible promoter GAL1p was introduced into W303-1A and W303-1B. The resultant recombinants were cultured in SG medium. Thereafter, the yields of prenyl alcohols (NOH and FOH) were determined (FIG. 21).

When HMG1 was introduced (the column at the left in each graph), the yields of both products increased. W303 clones were characterized by their effectiveness in the production of NOH.

### EXAMPLE 8

### Production of Prenyl Alcohols by High Expression of Deletion Mutant Type HMG-CoA Reductase Gene

In Example 7, prenyl alcohol-producing recombinant yeasts were developed using a full-length HMG-CoA reductase gene or a mutant thereof. In this Example, prenyl alcohol-producing recombinant yeasts were developed using a deletion mutant of HMG-CoA reductase gene, and alcohol production was carried out.

(1) Inducible Promoter; HMG1Δ; A451

The following plasmids (described in (4) in Example 3) obtained by inserting a deletion mutant of HMG1′ gene into a vector pYES2 comprising an inducible promoter GAL1p were introduced separately into A451.

pYHMG026

pYHMG044

pYHMG056

pYHMG062

pYHMG076

pYHMG081

pYHMG100

pYHMG112

pYHMG122

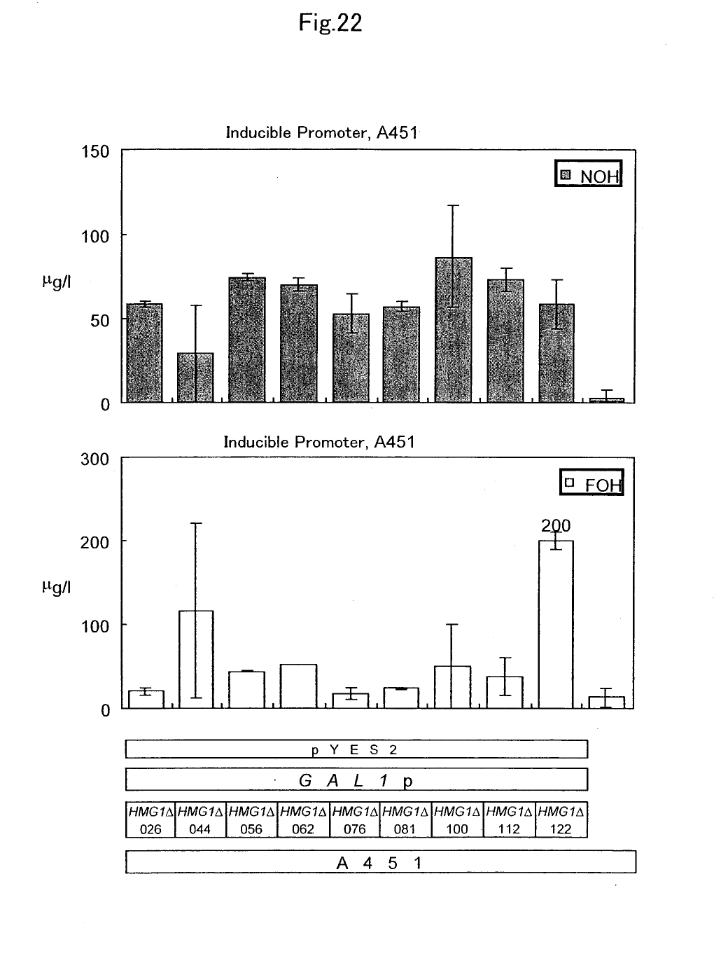

After completion of cultivation in SG medium, the yields of prenyl alcohols (NOH and FOH) were determined (FIG. 22).

When a deletion mutant type HMG1 gene was expressed with an inducible promoter, FOH high yielding clones were obtained. For FOH production, HMG1Δ044 and HMG1Δ122 were effective (FOH yield was 0.0 mg/L on the average in HMG1/A451 clones).

(2) Inducible Promoter; HMG1Δ; AURGG101

The following plasmids (described in (4) in Example 3) obtained by inserting a deletion mutant of HMG1′ gene into a vector pYES2 comprising an inducible promoter GAL1p were introduced separately into AURGG101.

pYHMG026

pYHMG044

pYHMG056

pYHMG062

pYHMG076

pYHMG081

pYHMG100

pYHMG112

pYHMG122

pYHMG133

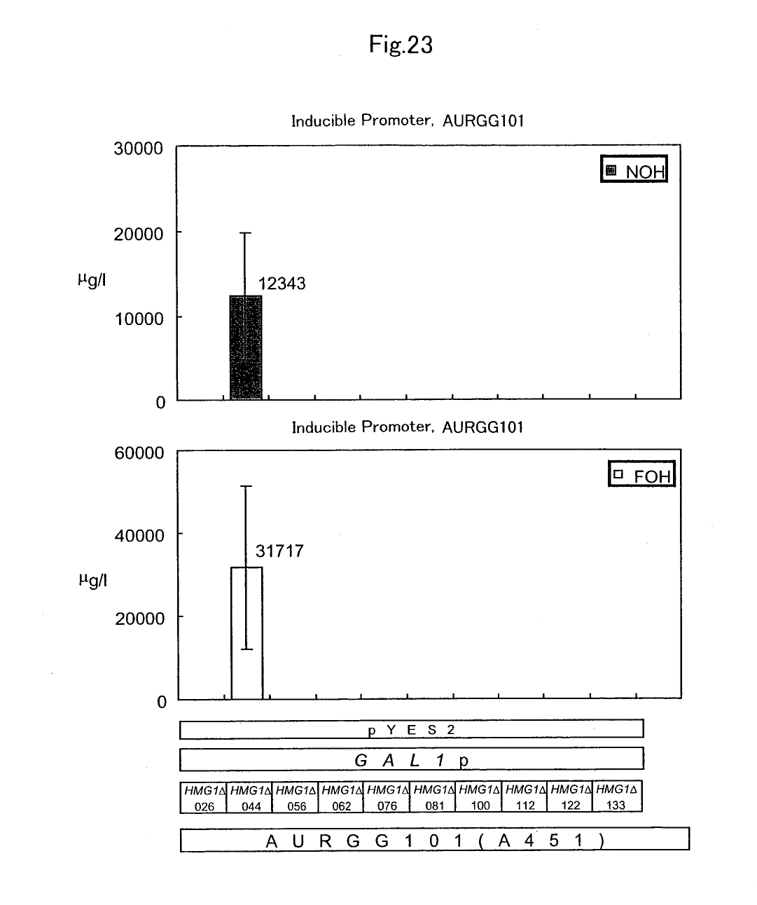

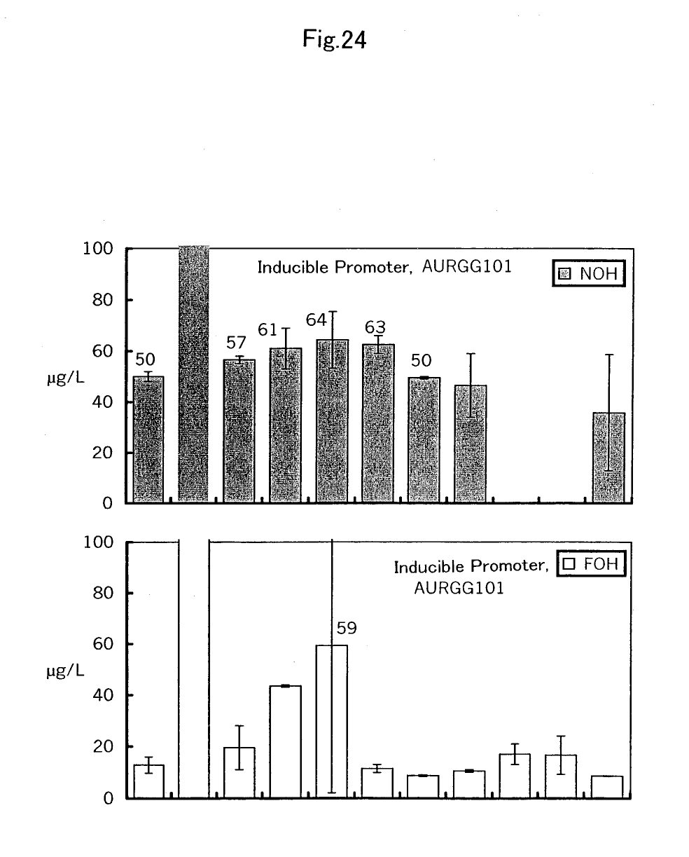

After completion of cultivation in SG medium, the yields of prenyl alcohols (NOH and FOH) were determined (FIGS. 22 and 23). In FIG. 23, the right utmost columns represent the yields of host AURGG101 before gene transfer. FIG. 24 shows enlarged graphs of FIG. 23.

In particular, when HMG1Δ044 was expressed with an inducible promoter, a prenyl alcohol high yielding clone (clone 15-2) was obtained. NOH yield and FOH yield in this recombinant were 12 mg/L and 31.7 mg/L on the average, respectively (FIG. 23). The maximum yields were 23 mg/L and 53 mg/L, respectively. In those recombinants integrating HMG1Δ other than HMG1Δ044, clones were obtained that produce NOH and FOH at about 0.05-0.06 mg/L (FIG. 24). The recombinant integrating HMG1Δ062 produced NOH at 0.11 mg/L and FOH at 0.13 mg/L at the maximum.

(3) Constitutive Promoter; HMG1, Inducible Promoter; HMG1Δ; AURGG101

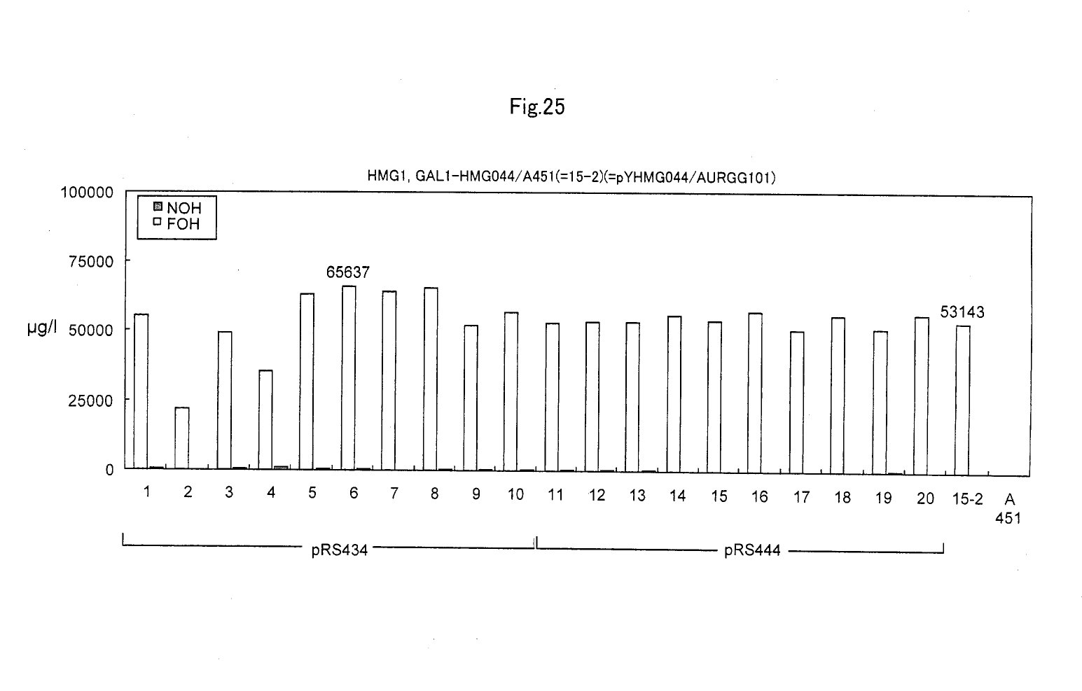

pRS434GAP-HMG1 or pRS444GAP-HMG1 prepared in (2) in Example 7 was introduced into clone 15-2 prepared in (2) above in this Example. After completion of cultivation in SG medium, the yields of prenyl alcohols (NOH and FOH) were determined (FIG. 25).

As a result, a clone was obtained that produced FOH at 66 mg/L at the maximum, improving the FOH yield of 53 mg/L of clone 15-2.

### EXAMPLE 9

### Production of Prenyl Alcohols by Escherichia coli

(1) The plasmids obtained in (4), (5) and (7) in Example 2 were introduced separately into E. coli JM109. To a 50 ml medium containing 2× YT and 1 mM IPTG in a 300 ml flask, 0.5 ml of a preculture was added. Antibiotics (ampicillin and chloramphenicol), if necessary, 5 mM (about 0.12% (w/v)) IPP and 5 mM DMAPP were added thereto, and the cells were cultured at 37° C. for 16 hr under shaking.

After completion of the cultivation at 37° C. for 16 hr, potato acid phosphatase was added to the culture supernatant and the cell pellet disrupted by sonication, followed by extraction of prenyl alcohols with pentane as an organic solvent. Then, the prenyl alcohols were identified and quantitatively determined by GC/MS as described in (1) in Example 7.

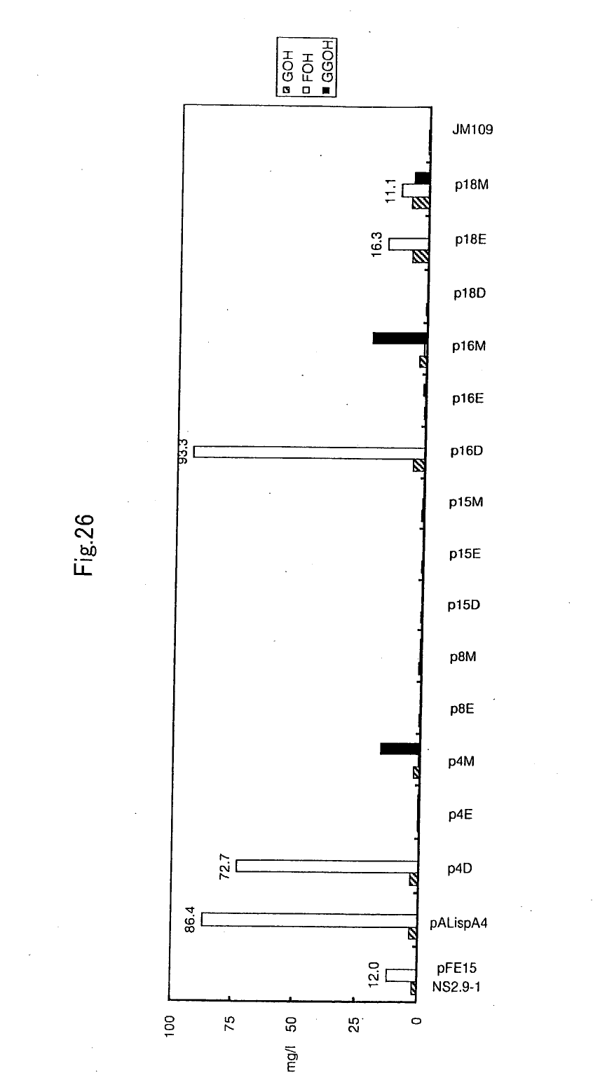

As a result, FOH yield in the presence of IPP and DMAPP was 86.4 mg/L when wild type ispA was introduced (pALispA in FIG. 29) and 12.0 mg/L when wild type fps was introduced (pFE15NS2.9-1 in FIG. 26). Even when a mutant ispA was introduced, JM109 retaining p18M or p18E produced FOH at 11.1 mg/L and 16.3 mg/L, respectively; JM109 retaining p4D produced FOH at 72.7 mg/L; and in JM109 retaining p16D, FOH yield reached 93.3 mg/L (FIG. 26).

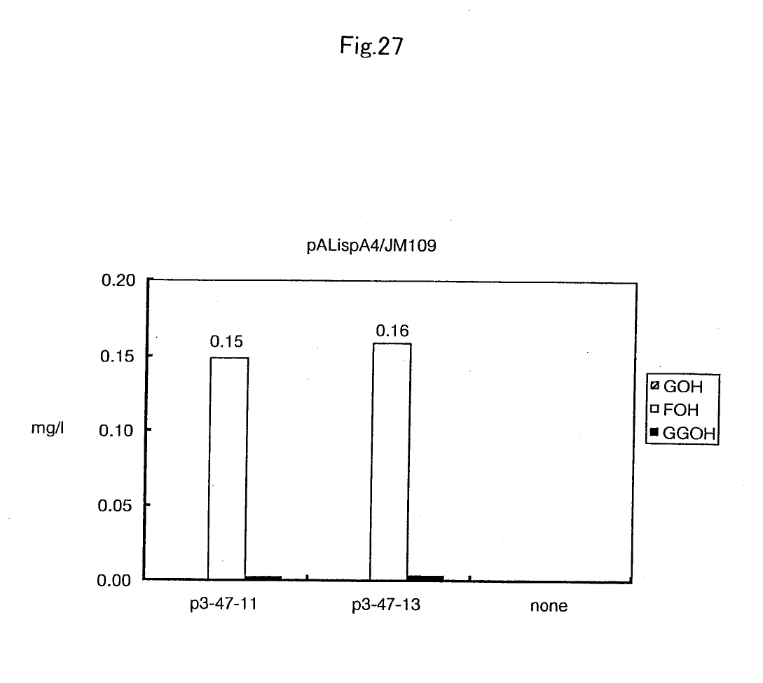

(2) In order to ascertain whether or not prenyl alcohol production can be carried out without the addition of IPP and DMAPP, plasmids pALispA4 and p3-47-11 or plasmids pALispA4 and p3-47-13 obtained in (4) and (6) in Example 2 were introduced into E. coli JM109. To a medium containing 50 ml of 2× YT per 300 ml flask and 1 mM IPTG, 0.5 ml of a preculture was added. Antibiotics (ampicillin and chloramphenicol) were added thereto, if necessary. Then, the cells were cultured at 37° C. for 16 hr under shaking. The results revealed that JM109 retaining pALispA4 and p3-47-11 has FOH production ability of 0.15 mg/L and that JM109 retaining pALispA4 and p3-47-13 has FOH production ability of 0.16 mg/L (FIG. 27).

Thus, it was found that E. coli retining plasmid p3-47-11 or p3-47-13 containing idi and plasmid pALispA4 containing ispA, i.e., E. coli incorporating idi and ispA has ability to produce FOH at 0.15-0.16 mg/L even without the addition of IPP and DMAPP.

### EXAMPLE 10

### Mass Production of FOH

1. Culture Conditions

One platinum loopful of the recombinant yeast clone 15-2 (AURGG101 retaining pYHMG044) described in (2) in Example 8 was inoculated from slants into CSM-URA medium (BIO 101 Inc.) and DOB medium (BIO 101 Inc.) (200 ml in a 500 ml Erlenmeyer flask with baffle plates) and cultured at 30° C. for 2 days under shaking at 130 r.p.m. Then, in order to remove the glucose contained in the culture broth completely, centrifugation (at 1500 g, for 5 min, at 4° C.) and washing with sterilized physiological saline were repeated 3 times. Subsequently, 50 ml of the culture was inoculated into a fermenter (1%) and cultured under the conditions described below.

Fermenter medium:

5% galactose

Amino acid-containing YNB (Difco)

1% soybean oil (Nacalai Tesque)

0.1% Adekanol LG109 (Asahi Denka)

Operational conditions:

Cultivation apparatus: MSJ-U 10 L Cultivation Apparatus (B. E. Marubishi)

Medium volume: 5 L

Cultivation temperature: 26° C.

Aeration rate: 1 vvm

Agitation: 300 rpm

pH: controlled proportionally using 4 N sodium hydroxide solution and 2N hydrochloric acid solution, and with the following parameters:

| | |
|---|---|
| Proportional Band | 1.00 |
| Non Sensitive Band | 0.15 |
| Control Period | 16 Sec |
| Full Stroke | 1 Sec |
| Minimum Stroke | 0 Sec |

2. Cell Counting

One hundred microliters of the culture broth was diluted 1- to 20-fold with physiological saline. Then, cells were counted with a hematometer (Hayashi Rikagaku). The number of cells in 0.06 mm² (corresponding to 9 minimum grids) was counted, followed by calculation of the average of 4 counts. Then, using the formula below, cell count per liter of the culture broth was calculated.

Cell count (1×10⁹/L broth)=0.444×(cell count in 0.06 mm2)×dilution rate

3. Quantitative Determination of FOH

FOH was identified and quantitatively determined in the same manner as in Example 8.

4. Results

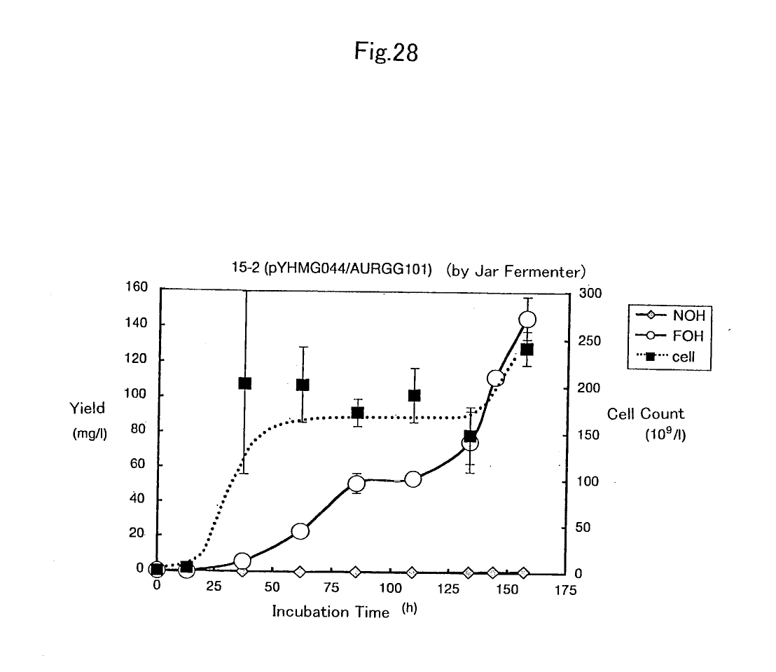

The results are shown in FIG. 28. As seen from FIG. 28, it was demonstrated that a recombinant yeast obtained by introducing HMG1Δ044 (a deletion mutant of the mutant type HMG-CoA reductase gene HMG1′) into A451-derived AURGG101 can produce 146 mg of FOH per liter of the culture broth on the average and 158 mg/L at the maximum.

All publications, patents and patent applications cited herein are incorporated herein by reference in their entirety.

Industrial Applicability

According to the present invention, a method of producing prenyl alcohols is provided. According to the present invention, biologically active prenyl alcohols can be obtained in large quantities. From these prenyl alcohols, isoprenoids/terpenoids with various physiological activities can be synthesized. The active prenyl alcohols provided in the invention may also be used as materials to find out those substances having a novel physiological activity.

SEQUENCE LISTING FREE TEXT
SEQ ID NOS: 18-74: synthetic DNA

1. A method of producing a prenyl alcohol, comprising creating a recombinant obtained by transferring into a host a recombinant DNA for expression or a DNA fragment for genomic integration each comprising:
(i) a hydroxymethylglutaryl-CoA reductase gene, an isopentenyl-diphosphate Δ-isomerase gene or a farnesyl-diphosphate synthase gene, or a mutant of any one of said genes,
(ii) a transcription promoter, and
(iii) a transcription terminator;
culturing said recombinant;
and recovering the prenyl alcohol from the resultant culture.

2. The method according to claim 1, wherein the prenyl alcohol is a C₁₅ prenyl alcohol.

3. The method according to claim 2, wherein the C₁₅ prenyl alcohol is farnesol or nerolidol.

4. The method according to claim 3, wherein the concentration of farnesol or nerolidol in the resultant culture is at least 0.05 mg/L.

5. The method according to any one of claims 1 to 4, wherein the hydroxymethylglutaryl-CoA reductase gene or mutant thereof comprises one nucleotide sequence selected from the group consisting of SEQ ID NOS: 1, 3, 5 and 7-16.

6. The method according to any one of claims 1 to 4, wherein the farnesyl-diphosphate synthase gene or mutant thereof comprises one nucleotide sequence selected from the group consisting of SEQ ID NOS: 75, 77, 79, 81 and 83.

7. The method according to any one of claims 1 to 4, wherein the isopentenyl-diphosphate Δ-isomerase gene or mutant thereof comprises the nucleotide sequence as shown in SEQ ID NO: 85.

8. The method according to any one of claims 1 to 7, wherein the transcription promoter is one selected from the group consisting of ADH1 romoter, TDH3 (GAP) promoter, PGK1 promoter, TEF2 promoter, GAL1 promoter and tac promoter.

9. The method according to any one of claims 1 to 7, wherein the transcription terminator is ADH1 terminator or CYC1 terminator.

10. The method according to any one of claims 1 to 9, wherein the host is yeast or Escherichia coli.

11. The method according to claim 10, wherein the yeast is Saccharomyces cerevisiae.

12. The method according to claim 11, wherein the Saccharomyces cerevisiae is A451 strain, YPH499 strain, YPH500 strain, W303-1A strain or W303-1B strain, or a strain derived from any one of said strains.

13. A recombinant obtained by transferring into a host a recombinant DNA for expression or a DNA fragment for genomic integration each comprising:
(i) a hydroxymethylglutaryl-CoA reductase gene, an isopentenyl-diphosphate Δ-isomerase gene or a farnesyl-diphosphate synthase gene, or a mutant of any one of said genes,
(ii) a transcription promoter, and
(iii) a transcription terminator,
said recombinant being capable of producing at least 0.05 mg/L of farnesol or nerolidol.

14. The recombinant according to claim 13, wherein the hydroxymethylglutaryl-CoA reductase gene or mutant thereof comprises one nucleotide sequence selected from the group consisting of SEQ ID NOS: 1, 3, 5 and 7-16.

15. The recombinant according to claim 13, wherein the farnesyl-diphosphate synthase gene or mutant thereof comprises one nucleotide sequence selected from the group consisting of SEQ ID NOS: 75, 77, 79, 81 and 83.

16. The recombinant according to claim 13, wherein the isopentenyl-diphosphate Δ-isomerase gene or mutant thereof comprises the nucleotide sequence as shown in SEQ ID NO: 85.

17. The recombinant according to any one of claims 13 to 16, wherein the transcription promoter is one selected from the group consisting of ADH1 promoter, TDH3 (GAP) promoter, PGKI promoter, TEF2 promoter, GAL1 promoter and tac promoter.

18. The recombinant according to any one of claims 13 to 16, wherein the transcription terminator is ADH1 terminator or CYC1 terminator.

19. The recombinant according to any one of claims 13 to 18, wherein the host is yeast or Escherichia coli.

20. The recombinant according to claim 19, wherein the yeast is Saccharomyces cerevisiae.

21. The recombinant according to claim 20, wherein the Saccharomyces cerevisiae is A451 strain, YPH499 strain, YPH500 strain, W303-1A strain or W303-1B strain, or a strain derived from any one of said strains.
